(19) United States
(12) Patent Application Publication
(10) Pub. No.: US 2013/0060125 A1
(43) Pub. Date: Mar. 7, 2013

(54) ENCEPHALOGRAPHY METHOD AND APPARATUS INCORPORATING INDEPENDENT COMPONENT ANALYSIS AND A SPECTRAL SHAPING FILTER

(71) Applicants: Philip Michael Zeman, Victoria (CA); Sunny Vardhan Mahajan, Victoria (CA)

(72) Inventors: Philip Michael Zeman, Victoria (CA); Sunny Vardhan Mahajan, Victoria (CA)

(73) Assignee: Applied Brain and Vision Sciences Inc.

(21) Appl. No.: 13/641,674

(22) PCT Filed: Apr. 15, 2011
(86) PCT No.: PCT/CA2011/050206

§ 371 Date: Oct. 16, 2012

Related U.S. Application Data
(60) Provisional application No. 61/325,164, filed on Apr. 16, 2010.

(51) Int. Cl. A61B 5/0476; A61B 5/05

(52) U.S. Cl. 600/409; 600/544

(57) ABSTRACT

Methods and apparatus for encephalography process encephalography data to yield components that correspond to modular areas of the brain. In some embodiments encephalography data is processed by independent component analysis to yield a first set of components that are processed to generate a spectral filter. The spectral filter is applied to the encephalography data to generate a second set of components. The second set of components may be represented by one or more matrices. In an embodiment the second set of components is represented by weight and sphereing matrices. Methods and apparatus may be applied for monitoring brain function, selecting participants for medical trials, adjusting drug dosages, performing and monitoring biofeedback methods, detecting progression toward diseases or conditions that affect the brain and other applications.

### CROSS-REFERENCE TO RELATED APPLICATION

This application claims priority from U.S. Application No. 61/325164 filed on 16 Apr. 2010 and entitled AUTOMATED DATA-DRIVEN METHOD FOR TRANSFORMING EEG OR MEG DATA INTO A BEHAVIORAL-ANATOMICAL-FUNCTIONAL MODEL AND SCHEMATIC REPRESENTATION OF INFORMATION PROCESSING which is hereby incorporated by reference herein. For purposes of the United States, this application claims the benefit under 35 U.S.C. §119 from U.S. Application No. 61/325164 filed on 16 Apr. 2010.

### FIELD OF THE INVENTION

This invention relates to encephalography and to processing encephalography data. Example embodiments provide methods and apparatus for use in testing and monitoring brain function, providing biofeedback, and/or classifying subjects according to brain function.

### BACKGROUND

Electroencephalography (EEG) and magnetoencephalography (MEG) are tools that may be used to detect signals arising from brain function. Encephalography signals result from the overall activity of the brain and also include signals arising outside the brain. There remains a need for practical methods and apparatus for applying encephalography to monitor and test brain function.

The following documents describe some techniques for studying brain function:
US 2010/0253905 published 7 Oct. 2010 to Lawton;
US 2010/0221688 published 2 Sep. 2010 to Reeves et al.;
US 2010/0235177 published 16 Sep. 2010 to Wischmann et al.;
US 2010/0240458 published 23 Sep. 2010 to Gaiba et al.;
US 2010/0249636 published 30 Sep. 2010 to Pradeep et al.;
US 2008/0003553 published 3 Jan. 2008 to Stark et al.;
US 2004/0092809 published 13 May 2004 to DeCharms;
US 2007/0027406 published 1 Feb. 2007 to LaPlaca et al.;
US 2007/0191704 published 16 Aug. 2007 to DeCharms;
US 2007/0254270 published 1 Nov. 2007 to Hersh;
WO 2010/123770 published 28 Oct. 2010 to Marci et al.;
EP 2031572 published 4 Mar. 2009 to Goldman et al.;
WO 2007/106518 published 20 Sep. 2007 to Sturm et al.;
WO 2009/137663 published 12 Nov. 2009 to Reichow et al.; and,
WO 2010/099443 published 2 Sep. 2010 to Forbes.

### BRIEF DESCRIPTION OF THE DRAWINGS

The accompanying drawings illustrate non-limiting embodiments of the invention.

FIG. 3 is a photograph showing a subject interacting with EEG apparatus according to a prototype embodiment.

FIG. 4 is a diagram including a simplified fabricated example scatter plot and trajectories obtained in iterations of a component analysis algorithm illustrating one way in which brain activity characterization of individual subjects can be used to characterize disease or dysfunction and/or classify the subject into a group (for example a stage of Parkinson's disease (PD) or Alzheimer's disease (AD).

FIG. 5 is a screen view of an example software interface as may be used in a client-server implementation of a data-cleaning service providing access to EEG or MEG data cleaning software that applies technology as described herein for estimating the anatomical location of detected artefacts and providing supervised artefact rejection.

FIG. 6 is a screen view of an example software interface as may be used in a client-server implementation of a brain activity data-mining service providing access to data-driven models of brain function.

FIG. 7 is a block diagram illustrating a data mining apparatus and data flow in a data mining method that applies spread spectrum independent component analysis (SS-ICA).

FIG. 8 is a block diagram illustrating apparatus and data flow in a method that may be used to process encephalography data using a pre-determined weight matrix and sphering matrix.

FIG. 9 is a block diagram illustrating apparatus and data flow in a method that may be used to process encephalography data using a pre-determined shaping filter.

FIG. 10 is a flow chart illustrating a method for generating a volume-domain representation for a component from coefficients defining the component.

FIG. 11 is a flow chart illustrating a method for modular source volumes.

FIG. 12 is a graph of the average percent change of validated component topographies as a function of the number of participant datasets mined to yield component topographies.

FIG. 13 is a set of charts showing example topographies corresponding to EEG components.

FIG. 14 is a graph showing average pair-wise correlation spectra for the non-shaped (black) and shaped (grey) decomposition methods.

FIG. 15 is a chart illustrating pair-wise correlation of components for various frequency bands for shaped and non-shaped methods.

FIG. 16 is a chart comparing pair-wise volume overlap (PVO), ranging from 0 to 1 for components common to shaped and non-shaped decomposition methods.

FIG. 17 shows example new components obtained by way of a shaped decomposition.

FIGS. 18A and 18B are two views of a head model with simulated sources.

FIG. 19 is a flow chart illustrating a method for constructing simulated encephalography data.

FIG. 20 shows original and recovered topographies and sources.

FIGS. 21A and 21B show respectively a recovered waveform when an extracted electrode artefact component is added to EEG data and a recovered topography.

FIGS. 22A and 22B respectively show recovered waveforms and recovered topographies for extracted ICA rank error components (7) and (8).

FIG. 23 shows localization and volume estimate results for five simulated neural sources.

FIG. 24 shows localization and volume estimate results for an electrode artefact, component.

FIG. 25 shows localization and volume estimate results for two rank-error components.

FIG. 26 shows estimated source volumes for 3 STDM to 6 STDM.

FIG. 27 shows several component topographies calculated using a runica algorithm. Positive field regions are represented by (+) while negative field regions are represented by (−). The field topology outside the head drawing depicts how the scalp field wraps around the sides of the head.

FIG. 28 shows source localization and volume estimation results for nine brain sources projected into a white-matter frame.

FIG. 29 is a graph showing source volume estimates for multiple STDM thresholds plotted as the volume estimate (number of voxels) versus the threshold used (number of STDM).

FIGS. 30A and 30B show convergence curves for iterations of a data mining algorithm. FIG. 30A shows convergence characteristics of the peak spectral value (PSV) over iterations calculated from synthetic EEG data for each component at each iteration. FIG. 30B shows the average and median calculated across components for each iteration.

FIG. 31 shows volumetric spectra of components superimposed to illustrate pair-wise overlap.

FIGS. 32A to 32C are convergence curves illustrating convergence characteristics of average volume overlap (AVO) and median volume overlap (MVO) over iterations calculated from synthetic EEG data. FIG. 32A shows convergence of AVO for each component. FIG. 32B shows convergence of the MVO for each component. FIG. 32C shows the average and median AVO calculated across components for each iteration.

FIG. 33 is a convergence curve showing movement of component centers of mass for each iteration of the runica maximization of statistical independence. Distance travelled between iterations is also shown.

FIG. 34 shows example ranking results for components calculated from synthetic EEG data.

FIG. 35 shows topographies of components calculated from real EEG data that were returned by runica source separation.

FIGS. 36A, 36B and 36C are convergence curves that respectively illustrate: convergence characteristics of the average volume overlap (AVO); median volume overlap (MVO); and the peak spectral value (PSV); of components over iterations calculated for real EEG data.

FIGS. 37A, 37B, 37C and 37D show convergence characteristics of each component as indicated in FIG. 37A by the peak spectral value (PSV) in FIG. 37B by the median volume overlap (MVO) in FIG. 37C by the total distance travelled by the center of mass in centimetres (TDT) and in FIG. 37D by the average volume overlap (AVO).

FIG. 38 shows ranking results for components calculated from real EEG data.

FIGS. 39A to 39D are views showing active modular brain volumes depicted on a white matter head model calculated from EEG data from a place/cue dataset.

FIG. 40 shows ensemble averaged instantaneous power as a function of time for activities of the brain volumes of FIG. 39D band pass filtered 3 to 40 Hz.

FIGS. 41A and 41B show two examples of the time-varying pair-wise zero-lag correlations in the frequency band 34 to 36 Hz relating to a subset of the brain volumes illustrated in FIG. 39.

FIGS. 42A to 42H show volume regions estimated for component source origins calculated from the EEG data of a participant group illustrated on a canonical cortex.

FIG. 43 is a comparison of RMS activation levels for the first second of spatial navigation for each component between the cue and place conditions.

FIGS. 44A and 44B show correlation of 8-30 Hz RMS brain activations with behavioural measures. FIG. 44A shows correlation of behavioural trial completion latencies with RMS activations of each component across study participants. FIG. 44B shows correlation of RMS brain activations with DS measures of explicit knowledge of platform location.

FIGS. 45A to 45D are scatter plots depicting RMS activations of a selection of paired components for the first second of spatial navigation demonstrating components that are correlated across study participants for one of the behavioural conditions.

FIGS. 46A and 46B are scatter plots depicting RMS activations of a selection of paired components for the first second of spatial navigation demonstrating components that are not correlated across study participants for both the cue and place conditions.

FIG. 47 is a block diagram of pair-wise correlation relationships calculated across the participant group (including possible outlier participant number 7) for the cue and place conditions.

FIG. 48 is a plot showing zero-lag correlation between components 29 (posterior parietal cortex) and 31 (superior parietal lobule) estimating coordination among brain areas (8-30 Hz).

FIG. 49 schematically illustrates coordination among brain areas measured via zero-lag correlation estimates for the interval around trial onset, −500 ms to 500 ms, 8-30 Hz instantaneous power.

FIG. 50 shows heat maps showing that eye-gaze position on the display screen differs between the ‘cue’ and ‘place’ conditions for the first second of navigation using the vMWT paradigm.

FIG. 51 illustrates results of a data-driven model of brain function created from data collected from persons while they navigated a computer-based virtual environment design to elicit cognitive spatial navigation strategies.

FIG. 52 is a schematic diagram illustrating the results of FIG. 51 where the areas of activation above baseline in the cue navigation task are indicated as blocks in the diagram and the relationships of coordination are indicated by lines connecting the blocks.

FIG. 53 is a block diagram illustrating processing encephalography data corresponding to events.

FIG. 54 illustrates results of a data-driven model of brain function created from data collected from persons while they navigated a computer-based virtual environment design to elicit cognitive spatial navigation strategies.

FIG. 55 is a block diagram illustrating an example general procedure for processing EEG or MEG data collected from one participant or from a group of participants.

FIG. 56 models effects of momentary correlations between source activities on source estimates.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

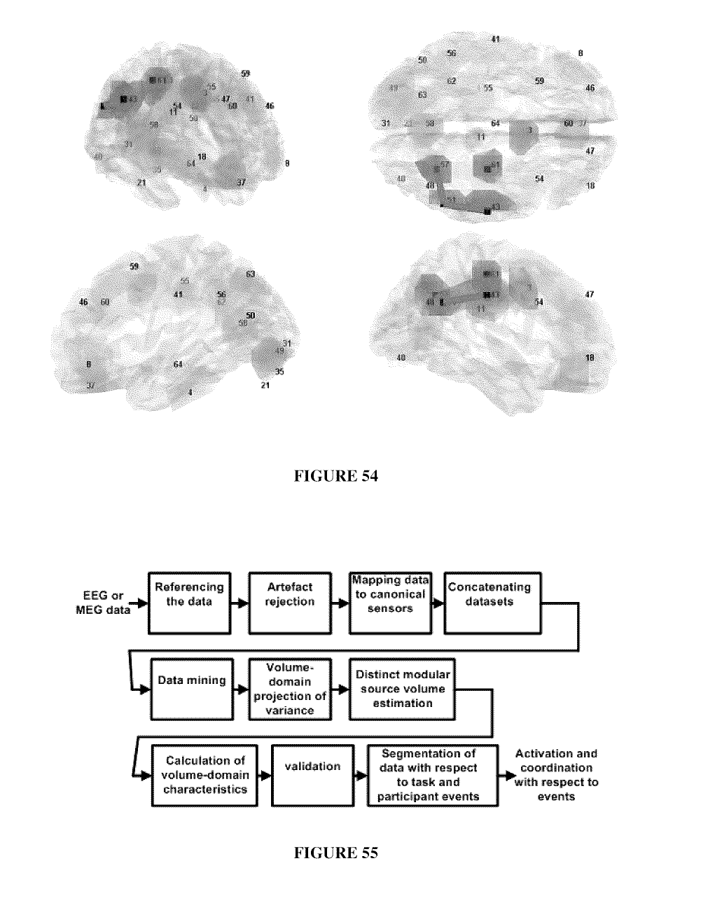

Models which relate brain function to encephalography data may be applied in a broad range of different applications. In an example embodiment, data is acquired from an individual while the individual is performing a task behaviour and the data is processed to provide information regarding the individual's brain function. The individual may be a person or an animal. In an example embodiment, the processing involves steps 1 to 10 as illustrated in FIG. 54. However, some of the steps illustrated in FIG. 54 are optional in certain applications and the illustrated procedure may be varied in other ways in some cases. Reference to ‘the method of FIG. 54’ in the following description encompasses all methods that have inventive features or combinations of features illustrated in FIG. 54.

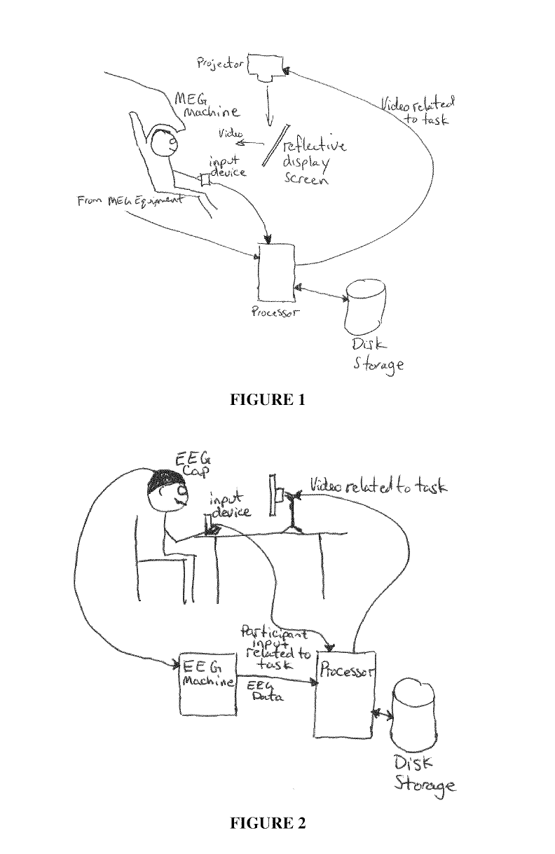

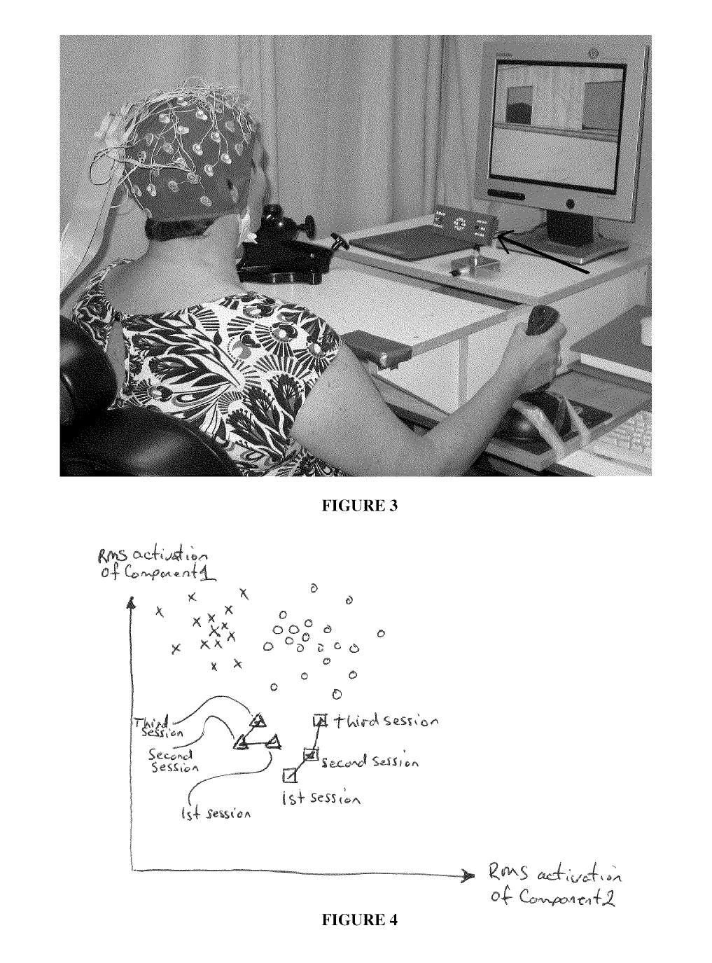

FIGS. 1 and 2 respectively illustrate example configurations of apparatus which may be used for acquiring MEG data and EEG data. FIG. 3 shows a prototype apparatus used in the development and testing of the disclosed technology. The apparatus includes: computers configured for executing the task software, collecting and storing EEG data and collecting and storing behavioural data as well as a joystick input device and a display screen for displaying stimuli. The arrow in the figure indicates an eye-tracking system. The subject is wearing a cap that carries a plurality of EEG electrodes. Electrical signals from the electrodes are amplified and recorded.

Some embodiments involve constructing a model indicative of brain function of one individual. The model may indicate changes in brain function states as the individual performs a task. The model may be applied to disagnose or classify the individual in relation to a disease or condition; to compare the individual to others (for example, for use in selecting or identifying a group of individuals who have similarities in brain function; to provide feedback to the individual for therapy, training or relaxation; to monitor changes in the individual's brain function over time or in response to a drug, therapy or other treatment; to assess the individual's fitness for certain tasks or the like.

EEG and/or MEG data can be collected from an individual in one recording session. During the recording session the individual may perform one or more tasks. The data may be processed, for example, using the general procedure of FIG. 54 to yield a set of validated components that relate to distinct areas of the brain.

EEG or MEG data for an individual may optionally be collected during multiple data collection sessions. In each session the individual may perform a different set of one or more tasks or the same set of one or more tasks. Data from multiple sessions may optionally be concatenated together and processed using the procedure of FIG. 54 to yield a set of validated components that relate to distinct areas of the brain.

In some embodiments encephalography data for an individual is analyzed with respect to a priori processed group data and an a priori determined model of brain function with respect to a task performed to determine relationships between the individual and the group.

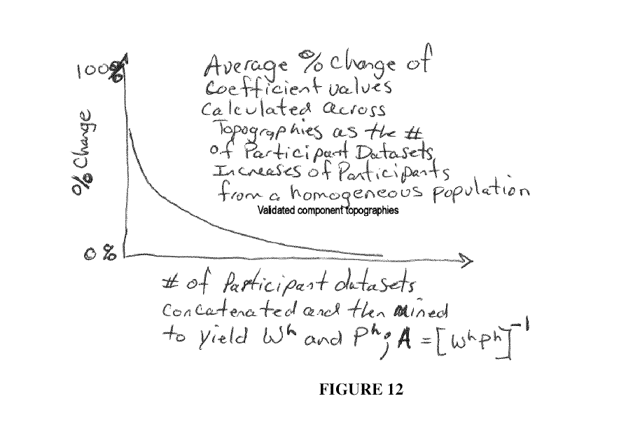

For example, EEG and/or MEG data can be collected from an individual and then processed according to a model calculated in advance based on data collected from a characteristic group of participants. For example, the participants on which the model is based could define a group representative of the population (the group may be very large in some cases). FIG. 12 shows that, as the number of participant datasets concatenated and processed by the data mining algorithm of the disclosed technology increases, the percentage change of the validated components topographies degreases.

The participants could optionally belong to a subgroup that has a characteristic brain disease, condition or dysfunction. In some embodiments the model comprises previously-determined sphering P^{h} and weight W^{h} matrices that may be applied to the encephalography data by multiplication, as described below. In some embodiments, processing encephalography data from an individual according to a model calculated in advance is performed in real time. Such real-time processing may indicate changes in activity of specific brain regions in real time.

EEG and/or MEG data collected from an individual may be combined and processed together with data collected from other individuals according to the method of FIG. 54 to yield an improved model relating encephalography data to specific brain regions. In some embodiments the EEG and/or MEG data is concatenated together for data mining.

The general procedure illustrated in FIG. 54 comprises the following steps.

### STEP 1: Referencing the Data

EEG and/or MEG data collected from each participant may be referenced. One method of referencing the data is to use an average of the data as a reference. Alternatively, the data can be referenced at infinity or de-referenced using the method described in “Van Veen B D, et al., Localization of Brain Electrical Activity via Linearly Constrained Minimum Variance Spatial Filtering. IEEE Transactions on Biomedical Engineering 1997;44:867-880” which is hereby incorporated herein by reference to obtain reference-free data.

### STEP 2: Preliminary Artefact Rejection

The EEG and/or MEG data collected from each participant may be processed by an artefact rejection algorithm to remove certain artefacts resulting from non-brain sources (e.g. artefacts resulting from muscle movements). The artefact rejection algorithm may be designed to leave any variance in the data that can not be demonstrated with high probability as belong to a non-brain source.

### STEP 3: Mapping Data to Canonical Sensor Positions

The EEG and/or MEG data from each participant can be mapped to a canonical sensor position configuration. This can be accomplished, for example, using a spline interpolation method or a wavelet interpolation method or some other method. While mapping to a common sensor position configuration can improve analysis results, it is not necessary if the sensors for each participant are all generally in the same place with respect to the individual brain anatomy of each participant. This step may optionally comprise defining a custom head model for each participant that has properties that minimize differences between participants related to variation of individual head and brain anatomy such as the size of the brain, the location and size of various lobes of the brain or other anatomical features of the brain. This head model may be used to minimize variability due to anatomical differences for data mining and between participant comparisons.

### STEP 4: Concatenating Participant Datasets

Depending on the intended application, data from an individual participant may be mined in isolation or data from a group of participants may be mined together. In some embodiments where data from an individual participant is to be mined, the data relating to all experimental conditions and tasks in the paradigm may be concatenated together to form a single concatenated EEG or MEG dataset to use in the data mining process.

If data from a group of participants are to be mined, then the data of all participants in the group relating to all experimental conditions and tasks in the paradigm may be concatenated together. This forms a single large multi-participant concatenated EEG and/or MEG dataset to be used in the data mining process.

### STEP 5: Data Mining to Identify Physically Distinct Brain Sources

The EEG and/or MEG dataset created in the previous step is mined with the intent of identifying components of the EEG or MEG data that represent the activities of distinct modular areas of the brain.

Mining the EEG or MEG data to identify components that represent the activities of distinct modular areas of the brain may be accomplished using any of a variety of methods, some of which include: independent component analysis (ICA)-based methods or other methods examining the statistics of signals second-order and above, or principal component analysis (PCA)-based methods or other methods that examine second order statistics. More generically, these methods may examine Gaussian and non-Gaussian mixtures for the purpose of identifying the sources contributing to the mixture.

Mining may be accomplished, for example, using the method of ICA called runica described by “Delorme A, Makeig S. EEGLAB: an open source toolbox for analysis of single-trial EEG dynamics including independent component analysis. J. Neuroscience Methods 2004;134:9-21. which separates data into statistically independent parts to identify component parts, with each component part having a corresponding time-varying waveform and topography.

The runica algorithm assumes that the sources comprising the data mixture are statistically independent and spatially stationary. The general mathematical model for the runica method of ICA is given in the following equations.

The general mixture model for the runica method of ICA is given by Equation 1,

x=As (1)

where x is the observed mixture and s describes the sources comprising the mixture. The matrix A of scalar values, describes the physical characteristics of the mixture. Each row of A determines how the sources combine to form a particular observed signal, and each column of A determines how a particular source is distributed among the observed signals. Both A and s are generally not known.

The observed mixture x contains K rows of spatially distributed, time-domain sensor measurements (Equation 2), while the source matrix s contains J rows of unknown time-domain sources activities (Equation 3). There should be at least as many EEG electrodes or MEG sensors as there are unknown sources, such that K≧J. In general, it is assumed that each row of x has zero mean. If this is not the case, this can be accomplished by subtracting the mean of each row of x.

x=└x₁(t), x₂(t), . . . x_{K}(t)^{T} (2)

s=[s₁(t), s₂(t), . . . s_{J}(t) (3)

Given that the assumptions about the sources stated above are satisfied, the problem reduces to the task of finding a linear transform, W, as outlined in Equation 4. The weight matrix W, linearly reduces the mixture into parts and is used by Equation 5 to solve for the mixing matrix A, and the scalp topographies of each source held in the columns of the mixing matrix.

s=Wx (4)

A=W⁻¹ (5)

Equations 4 and 5 can be expanded to include a step of data sphering that is used in the runica algorithm. In Equations 6 and 7, Equations 4 and 5 are expanded to include the sphering matrix P. Multiplication of the data by the sphering matrix P transforms each of the rows of x to have unit variance and to be uncorrelated from all other rows comprising x. This step is sometimes referred to as whitening. The sphering matrix, also known as the whitening matrix can be calculated as the inverse of the matrix-square-root of the data covariance matrix. The sphering or whitening matrix may also be calculated as a constant of 2 multiplied by the inverse of the matrix-square-root of the data covariance matrix.

s(t)=WPx (6)

A=(WP)⁻¹ (7)

In summary, the general steps in the runica algorithm include: (1) subtract the mean from each channel of data, (2) compute a sphering matrix P corresponding to the data and multiply the data by the sphering matrix, (3) solve for W using a minimization or learning algorithm, and (4) compute the mixing matrix A. From the mixing matrix A, the topographies of components of the data may be derived. From the source matrix s, the time-varying waveforms corresponding to the topographies may be derived.

The runica algorithm is an iterative algorithm that, through successive iterations, converges on a final resulting weight matrix, W via the minimization or learning algorithm that is used. Hence, the mixing matrix A, and the corresponding topographies contained in A, can be determined on each successive estimate of W. This facilitates the possibility that each component being estimated could undergo a validation process on each successive estimate of W. In some embodiments such validation processes are performed on a plurality of iterations of runica (or another iterative algorithm applied to determine source values).

Mining can also be accomplished using the runica algorithm performed in conjunction with an analysis method that is spectrally selective. For example, runica or another suitable ICA algorithm may be combined with Band-Selective ICA (BSICA) as described, for example, in Zhang, K. Chan, L. W. An Adaptive Method of Subband Decomposition ICA, Neural Computation, 18(1), 2006, pp. 191-223. In one such embodiment, BSICA is applied as a post-processing step for the runica algorithm to calculate the mixing matrix A and the matrix W. This combination of runica and BSICA makes the assumption that there is some statistical dependence between sources in some frequency band(s). However, it does not make the assumption that some sources are statistically dependent and correlated in some frequency band.

Mining can be accomplished using a method which assumes that the sources are correlated in some frequency bands and uncorrelated and statistically independent in other frequency bands and uses these attributes at least in part, to separate the data into component parts with each component part having a corresponding time-varying activity and topography.

Mining can be accomplished using a method that separates the EEG mixture into parts based on the activities in the frequency bands of least correlation and statistical dependence. In such embodiments, a mining algorithm may adaptively search for and identify those frequencies of least statistical dependence and uses the frequencies identified to separate the data into components with each component having a corresponding time-varying activity and topography that relates to physical modular volumes of the brain.

Mining can be accomplished using “Spectral Shaping ICA” or “Spectral Shaping Methodology” as described below. Such mining may apply the runica algorithm and BSICA algorithm in a manner that produces a result (such as a mixing matrix A) that is different from the results that would be obtained by application of the method described in Zhang, K. Chan, L. W. An Adaptive Method of Subband Decomposition ICA″, Neural Computation, 18(1), 2006, pp. 191-223 and is also different from the results that would be produced by either of BSICA or runica used independently. Embodiments that apply Spectral Shaping ICA may use the runica algorithm or other methods of ICA to separate data into statistically independent components. Similarly, such embodiments may apply BSICA or other algorithms that identify frequencies of statistical independence or correlation.

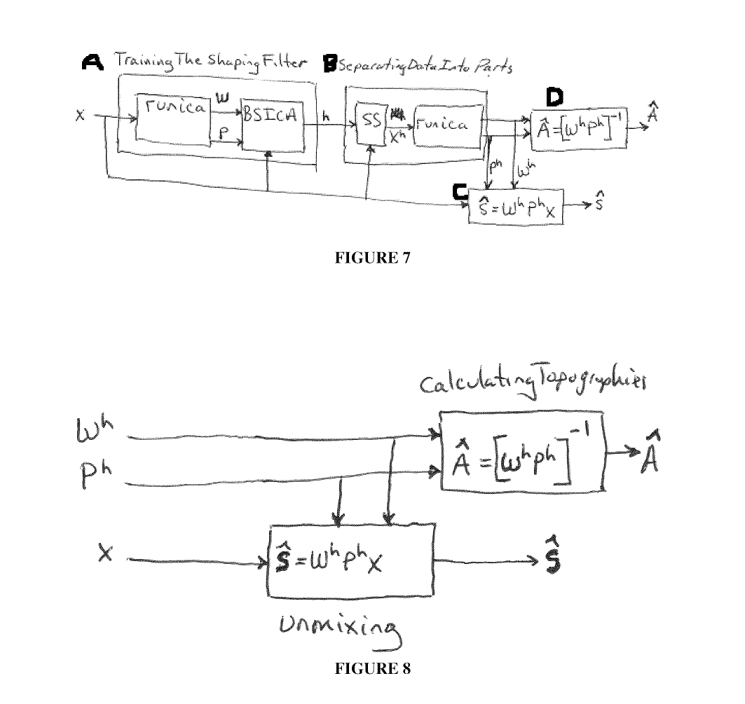

An example Spectral Shaping ICA embodiment exploits the complementary properties of runica and BSICA and is configured as illustrated in FIG. 7. In the following description, it is assumed that the data, denoted as x, have zero mean on the sensor channels. In FIG. 7, ‘runica’ denotes use of the runica algorithm; BSICA denotes use of the BSICA algorithm; ‘SS’ denotes shaping of the EEG frequency spectrum using calculated filter coefficients; the input data symbolized by x in the figure represents EEG or MEG data where the mean of each channel of data has been subtracted. The input could be: from an individual person, from a group of persons with each individual's data concatenated together to form one large dataset. Block (a) represents training of the spectral shaping filter. Block (b) represents calculation of the separation matrices for the brain activity. Block (c) represents calculation of estimated source activities. Block (d) represents calculation of topographies of components of the EEG.

In FIG. 7, a shaping filter that emphasizes frequencies of independence (de-emphasising frequencies of dependence) is calculated through a combination of runica and BSICA. Next, the data x are filtered and shaped using the calculated shaping filter and then used to define sphering and weight matrices via a second usage of runica. The sphering and weight matrices calculated in this way are denoted as P^{h} and W^{h}, respectively, with the superscript h indicating they result from the filter-shaped data. An example of the calculation of estimates of source activities ŝ by this method is given in Equation 8,

ŝ=W^{h}P^{h}x (8)

where x is the non-shaped EEG data with the mean of each channel removed.

Similarly as in Equations 4 and 6, the estimate of the mixing matrix, Â is calculated as in Equation 9, however, in this case, both the weight and sphering matrices are modified by the shaping filter.

Â=(W^{h}P^{h})⁻¹ (9)

There are 4 main processing steps, presented as 4 main processing blocks in FIG. 7. Block (a) provides training of the shaping filter and may involve two parts. First, the an ICA method such as runica is used to calculate a preliminary weight matrix W and a sphering matrix P from the zero-mean EEG data, x. These spatial unmixing matrices W and P are inputs to the BSICA block. The BSICA algorithm is used to calculate filter coefficients, h. The coefficients of h emphasize frequencies of statistical independence relative to frequencies of dependence. Block (b) separates the brain activities from the EEG mixture, calculating new spatial unmixing matrices P^{h} and W^{h}. Using h to shape the spectra of x, (denoted in the figure as ‘SS’) the dataset x^{h} is created and statistical independence is maximized by runica on x^{h}. This creates the new sphering matrix P^{h} and weight matrix W^{h}. To calculate estimated source activities ŝ, the matrices P^{h} and W^{h} are combined with the unshaped EEG data x in block (c). In block (d), the matrix of estimated brain source topographies, Â is generated from P^{h} and W^{h}. Thus, the matrix Â contains the topographies of each component of the data and the corresponding component waveforms are contained in the new source matrix ŝ.

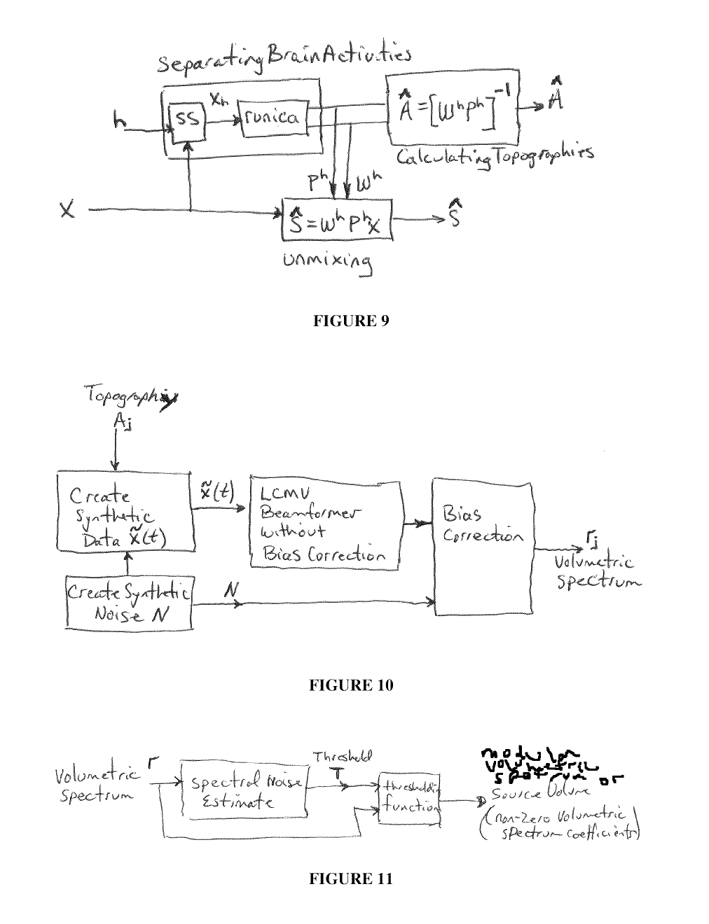

In an alternative embodiment, the shaping filter h, is determined in advance (for example based on analysis of previously-acquired encephalography data for a population or individual), thus reducing the complexity of the data mining procedure. This is illustrated in FIG. 9. In FIG. 9, input data symbolized by x represents EEG or MEG data where the mean of each channel of data has been subtracted. The output matrix A^{h} contains the topographies of each component while the matrix s^{h} contains the time-varying activities of each component. The symbol ‘SS’ denotes shaping of the EEG frequency spectrum using the calculated filter coefficients.

In an alternate embodiment, the sphering P^{h} and weight W^{h} matrices, are pre-calculated and pre-multiplied for doing analysis of new participant datasets with respect to data already provided by a group of participants. This is illustrated in FIG. 8. Ideally the data used to create P^{h} and W^{h} is created from a dataset of multiple persons large enough to encompass what sources might appear in the data from new individual persons or groups of persons. The input data symbolized by x in the figure represents EEG or MEG data where the mean of each channel of data has been subtracted. The configuration of FIG. 8 may also be used with a beamformer (such as illustrated for example in FIGS. 10 and 11) to show brain function (for example, activation and coordination) at specific areas of the brain in real-time or pseudo-real-time. This could be used in a neurofeedback application or in a diagnostic applications.

In an alternative embodiment, the estimates of the sphering P^{h} and weight W^{h} matrices (and the matrix Â) on each successive iteration of the minimizing or learning process in the data mining algorithm are provided as output. Hence, from Â the topographies corresponding to each component (at the current iteration) can be determined. This embodiment has the provision of enabling the calculation of volumetric characteristics of each component during the data mining process.

In some embodiments trajectories of components or characteristics derived from components as an iterative algorithm in the data mining process converges are used to identify diseases or conditions such as Alzheimer's Dementia or Parkinson's Dementia at an early-stage. In some embodiments, trajectories of components or characteristics derived from components are applied to validate the components and/or distinguish artefacts from components that correspond to modular regions of the brain.

STEP 6: Projection of Topographical Variance into Volume-Domain Representation

Validation of the data mining results can be accomplished, for example, by evaluating the quality of each component yielded by the data mining algorithm as representative of a distinct volume of the brain. Validation of components using volume domain characteristics, where the components were identified via ICA-based processes evaluating waveform activities, is effective for two reasons: (1) the assumptions of the data mining algorithm do not include volume-domain characteristics and are hence separate from the criteria by which data mining results are validated, (2) it is of interest to identify those components that are good estimates of brain source activities because they are the basis for the model of brain function that we are constructing through the use of the processing steps described.

Projection of the topographical characteristics of each component into a volume-domain equivalent can be done at each step or selected steps of the data mining process or at the completion of the data mining process.

Projection of the topographical characteristics of each component into a volume-domain equivalent can be accomplished, for example, using a beamforming approach where the topographical variance is represented in a 3D space head model. The beamformer used could be a Linearly Constrained Minimum Variance (LCMV) beamformer, for example. An LCMV beamforming approach may be applied to represent the topographical variance of components in a 3D space head model

One suitable LCMV beamforming approach is described in “Van Veen BD, et al. Localization of Brain Electrical Activity via Linearly Constrained Minimum Variance Spatial Filtering. IEEE Transactions on Biomedical Engineering 1997; 44:867-880” where the topographical variance is represented in a 3D space head model. This LCMV beamforming method may be implemented using the equations below to both separate time-varying EEG data into uncorrelated (not necessarily statistically independent) components and identify center locations of each uncorrelated component within a head model. The mathematics of the LCMV Beamformer link time-varying scalp-EEG data to a volume domain representation. The LCMV Beamformer utilizes the measured covariance between scalp electrodes to provide an estimate of the volume-projected variance at all points within a model head.

Equation 10 gives a noiseless forward model for a single source location qₚ where H(qₚ) is the K×3 (a separate vector for each x, y, and z direction) transfer matrix from locations inside the head to the surface of the scalp x, and m(qₚ) is the 3×1 vector representing the dipole moment at a given location. There are a total of P grid locations inside the head model for which to assign variances from K scalp electrodes.

x=H(qₚ)m(qₚ) (10)

For the case of multiple locations or voxels within the head model we can utilize the summation of Equation 11 where x is now the superposition of multiple dipoles projecting to the scalp.

$$x = {\sum }_{p = 1}^{P} H ( {q}_{p} ) m ( {q}_{p} ) \qquad ( 11 )$$

Equation 12 describes a mathematical model that relates the observed covariance, C(x), at the electrodes to the covariance, C(qₚ), at each location within the head model, and to the head model transfer matrix.

$$C ( x ) = {\sum }_{p = 1}^{P} H ( {q}_{p} ) C ( {q}_{p} ) {H}^{T} ( {q}_{p} ) \qquad ( 12 )$$

The sum of x, y, and, z variances for a dipole moment of a location, qₚ, inside the model head, is defined in Equation 13,

VAR{qₚ}=tr{C(qₚ)} (13)

where VAR{qₚ} denotes the total variance at location qₚ and tr{C(qₚ)} is the trace of the covariance matrix at location qₚ.

To isolate C(qₚ) we exploit the relationship between the covariance matrix C(x) and the transfer matrix H(qₚ) for each location qₚ to solve the inverse problem illustrated in Equation 16. To do so, Equations 14 and 15 may be utilized where m̅(qₚ) is the expected value of the moment at a given location qₚ and E is the expectation operator.

m̅(qₚ)=E{m(qₚ)} (14)

C(qₚ)=E{[m(qₚ)− m̅(qₚ)][m(qₚ)− m̅(qₚ)]^{T}} (15)

The solution to the inverse problem (Equation 16) may be obtained using a K×3 matrix V that defines a pass-band and stop-band based on C(x) for the head model characterized by H.

m(qₚ)=V^{T}x (16)

The solution for V may be provided as Equation 17.

V(q₀)=[H^{T}(qₚ)C⁻¹(x)H(qₚ)]⁻¹H^{T}(qₚ)C⁻¹(x) (17)

Substituting Equations 15 and 16 into 13 yields Equation 18,

VAR{qₚ}=tr{┌H(qₚ)C⁻¹(x)H(qₚ)┐⁻¹} (18)

the projection of scalp variance to volume domain variance for a specific location qₚ.

Since sensors are not usually equidistantly from each other placed in a sphere around the head (allowances for face, neck, and torso must be made), the LCMV beamformer suffers from a bias due to electrodes being placed in a hemisphere on the scalp of the head.

The LCMV method of beamforming compensates for non-uniform electrode spacing by collecting a “noise” sample from the EEG or MEG. This “noise” sample is typically an EEG or MEG baseline where the brain function of interest is not present in the data. In providing compensation for non-uniform sample, Equation 18 is replaced by Equation 19 as in the Equation below where the covariance matrix of the “noise” sample in this case denoted by Q is the denominator of the right-hand side of the equation.

$$VAR \{ {q}_{p} \} = \frac{tr \{ {[ {H}^{T} ( {q}_{p} ) {C}^{- 1} ( x ) H ( {q}_{p} ) ]}^{- 1} \}}{tr \{ {[ {H}^{T} ( {q}_{p} ) {Q}^{- 1} H ( {q}_{p} ) ]}^{- 1} \}} - 1 \qquad ( 19 )$$

The volumetric spectrum comprised of the variances at each location inside the head model for a given C⁻¹(x) and noise estimate or baseline condition is given as Equation 20,

r=[VAR{q₁}, VAR{q₂}, . . . , VAR{qₚ} (20)

where each voxel in the head model is specified as q₁, q₂, . . . , qₚ.

The topography of each component derived at the completion of the data mining process or at each iterative step as the data mining process converges toward completion can be transformed into a volume-domain equivalent representation using the LCMV beamformer and in conjunction with synthetic data derived from the topography. This may be accomplished through the steps described below.

First the sum of the sensor weights defining the topography is calculated and subtracted from each sensor weight defining the topography. Alternatively, the topography can be de-referenced using the method described in “Van Veen B D, et al. Localization of Brain Electrical Activity via Linearly Constrained Minimum Variance Spatial Filtering. IEEE Transactions on Biomedical Engineering 1997; 44:867-880 to obtain a reference-free topography. After this step, the topography may be normalized to have unit Euclidian norm.

A synthetic source signal is then created using the normalized topography indicated as Âⱼ in the equations below where j is an index for the topography.

Synthetic data \tilde{x}(t) are created with known second order stationary statistics using Equation 21,

\tilde{x}(t)=Âⱼ^{T}G+N (21)

where G is a Gaussian process of dimension 1×M and N is a white noise process of dimension K×M with exactly zero correlation among the electrodes and zero correlation among the dimensions of G and N. The number of time-domain samples is given by M (40,000 samples in an example implementation, however a different number can be used). The Gaussian sequence G is zero-mean and has unit standard deviation σ_{G} on each electrode. The noise process N is zero-mean and is scaled such that it has a standard deviation, σ_{N}=0.0001σ_{G}. Alternatively, a different scale factor could be used to scale the noise process. This ratio provides separation of the noise used to estimate bias and the projected topography. Numerically, it is chosen to allow the low power, uncorrelated additive noise N to be separated from the Âⱼ^{T}G term via Equation 21.

This zero-correlation relationship between G and N can be created, for example, by combining N and G in a single matrix that is then sphered to remove partial correlation. Once sphered, N and G can be extracted by position. For example, using Equation 22, an arbitrary matrix y may be created by concatenating G and N such that their new dimension is K+1×M. Once sphered by the inverse of the covariance matrix of y, the result ŷ is created. After sphering, all columns of y have exactly zero correlation.

ŷ=└C^{-1/2}(y)┘y (22)

The synthetic data \tilde{x}(t) may then be processed to solve for the numerator of Equation 19. The matrix Q⁻¹ of Equation 19 is calculated as the inverse covariance matrix of the additive noise component N. This combination yields a bias-corrected LCMV beamformer result that when used in Equation 20 is a vector containing the coefficients of the volumetric spectrum.

In one embodiment, the values in the matrices G and N are pre-calculated to have zero-correlation relationship between each other to reduce the computations required to calculate the volumetric spectra of multiple topographies.

Alternatively, a different approach that projects the topographical variance of each component of the data mining step into a volume-domain head model using a beamformer or an LCMV beamformer approach may be used to provide a closed-form solution without requiring creation of synthetic data.

FIG. 10 is a block diagram illustrating one way to implement transformation of each topography calculated in a data mining process from coefficients stored in Aj (where j is the index of the topography corresponding to component number) to coefficients describing the topography as a volume-domain representation. The output r j is the volumetric spectrum corresponding to the topography.

### STEP 7: Modular Source Volume Estimation

The modular source volume equivalent of each scalp topography can be generated by applying a threshold to each volumetric spectrum calculated in the previous step.

This threshold can be calculated as in Equation 23 where T is the threshold level, such that any volumetric spectrum coefficients that are below the threshold are assumed to be noise and are set to be zero-valued. The threshold, T can be determined as in Equation 23:

T=Rσᵣ+ r̅ (23)

where r̅ is the mean of the volumetric spectrum, R is a user-specified constant (referred to as STDM) and σᵣ is the standard deviation of the volumetric spectrum. In an example embodiment the user-defined parameter R is usually set to 4 or 5 for a head volume with 5439 voxels. This value for R was empirically determined using model data to provide a suitable representation of volume.

This method of thresholding is illustrated in FIG. 11. FIG. 11 is a flow chart illustrating the calculation of the component volume-domain representation edge boundaries for the purpose of defining a physically modular source volume. The value, r corresponds the volumetric spectrum, T is the threshold value used by the thresholding function to set all coefficients of the volumetric spectrum that are below the threshold value. The output is the modular volumetric spectrum which may be referred to as the “source volume”.

The modular source volume equivalent of each scalp topography is defined by those voxels that have not been set to be zero-valued.

Alternatively, another method of estimating the source volumes from the volumetric spectrum can be used.

### STEP 8: Calculation of Volume Domain Characteristics of Components

Various volume-domain characteristics may be derived from the volumetric-spectrum of a component topography. Values for such characteristics may can be calculated either at the completion of the data mining process or at each successive iteration of the data mining process for the purpose of showing the convergence of volume-domain characteristics of each component.

Example volume-domain characteristics are the Peak Spectral Value (PSV), Average Volume Overlap (AVO), Average Volume Overlap2 (AVO′), Median Volume overlap (MVO), and the Median Volume Overlap2 (MVO′). If volume-domain characteristics are being determined for successive iterations of the data mining process, then an additional characteristic called the Distance Travelled (DT) may be calculated.

PSV may be set equal to the largest value of the volumetric spectrum (or to some other value that provides an indication of the intensity of the volumetric spectrum such as the 98^{th} percentile of the volumetric spectrum, the average of the N largest values in the volumetric spectrum or the like).

The values of MVO, AVO may be calculated from the estimate of volume overlap given in Equation 24 which estimates volume overlap S of pairs of i,j components.

$${S}_{ij} = \vert \frac{( {r}_{i} - {\overline{r}}_{i} ) {( {r}_{j} - {\overline{r}}_{j} )}^{T}}{‖ ( {r}_{i} - {\overline{r}}_{i} ) ‖ ‖ ( {r}_{j} - {\overline{r}}_{j} ) ‖} \vert for all i \neq j \qquad ( 24 )$$

where vectors rᵢ and rⱼ are the volumetric spectra and r̅ᵢ and r̅ⱼ are their respective means. Each vector has 1×P elements, where P is the number of spectral coefficients representing the variance in the head volume. The overlap ranges between 0 and 1 indicating no overlap and maximum overlap, respectively.

The AVO may be calculated as the sum across Sⱼ for all i≠j, while holding j constant and then divided by the number of values in the summation. This may be repeated for each component j and iteration of runica. The MVO may be calculated as the median of Sⱼ for all i≠j while holding j constant and then repeated for each j component.

To obtain values for AVO′ and MVO′, the ‘volume overlap absolute’ is used as calculated as shown in Equation 25. The volume overlap absolute has the advantage of avoiding some instabilities that can be associated with the first overlap estimation method. For this second method of measuring overlap, the means are not subtracted from vectors rᵢ and rⱼ prior to scaling to unit Euclidian norm. This is illustrated in Equation 25,

$${S}_{ij}^{′} = \vert \frac{( {r}_{i} ) {( {r}_{j} )}^{T}}{‖ ( {r}_{i} ) ‖ ‖ ( {r}_{j} ) ‖} \vert for all i \neq j \qquad ( 25 )$$

where the pair-wise overlap is designated as S′ᵢⱼ. As in Equation 24, a result of 0 indicates that the two spectra compared have no volume-domain overlap while a 1 indicates they have perfect volume-domain overlap. From the volume overlap absolute from Equation 25, the MVO′ and AVO′ may be created following the same steps as described above for the MVO and the AVO.

The distance travelled (DT) may be calculated as the three-dimensional x-y-z position of the spatial coefficient with the largest value (the coefficient containing the PSV) in the current successive iteration of the data mining process compared with the x-y-z position in a previous iteration. This is described in Equation 26,

DTₖ=\sqrt{(xₖ−xₖ₋₁)²+(yₖ−yₖ₋₁)²+(zₖ−zₖ₋₁)²} (26)

where k varies from 2 to the total number of iterations. For iteration number 1, the starting position of comparison is defined to be 0, 0, 0. Alternatively, the starting point can be related to the topography and equivalent volumetric spectrum generated from the sphering matrix used in the data mining algorithm.

Calculations of the volume-domain characteristics of the components determined at the completion of the data mining process is the same those calculations used during the data mining process. However, there is an additional characteristic to calculation which is called the Total Distance Travelled (TDT). This characteristic calculated as the sum of the DT values for each component separately over the data mining process.

In another embodiment, the topographies corresponding to each successive iteration of the data mining process are stored. Once data mining has completed, the volume domain projection and corresponding volume domain characteristics of each topography can be calculated to generate curves of convergence.

### STEP 9: Validation of Components Found by Data Mining

Validation of components being representative of distinct brain sources may be done in two parts. The first part is a qualitative view of the relative convergence characteristics of each of the components identified, while the second part is an automated selection of which components are characteristic of distinct brain sources. In theory, the user of the algorithm always has the option to reject specific components based on component convergence characteristics, however, multiple uses of this methodology has shown that generally the qualitative convergence results reflect the quantitative final results and that no user input in the validation procedure is required.

In another embodiment, the characteristics of convergence of one or more of the PSV, MVO, MVO′, AVO, AVO′ and/or other characteristics of components found in the data mining process are combined with the final results to generate improved quantitative characteristics for validating components.

### Qualitative Evaluation: Convergence of Volume-Domain Characteristics

The step of qualitative evaluation of components provides the user with confidence that the components generated by the data mining algorithm and the subset of components approved by the validation algorithm as being likely representatives of distinct brain areas provides the user of the algorithm with confidence in the results. It essentially provides the user with a ‘look inside the black box’ of processing so that they can see that final result at which was arrived makes sense.

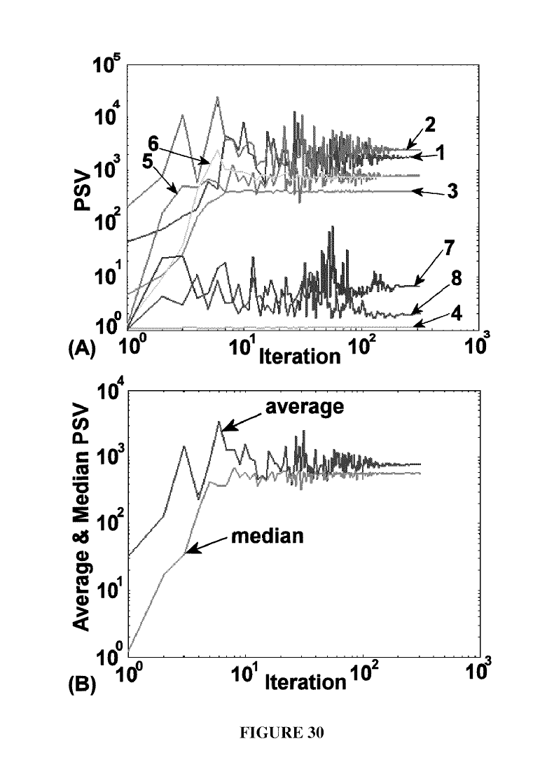

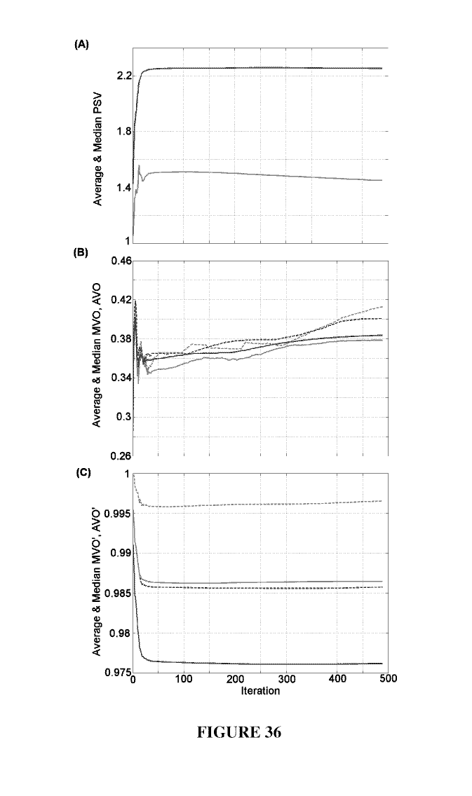

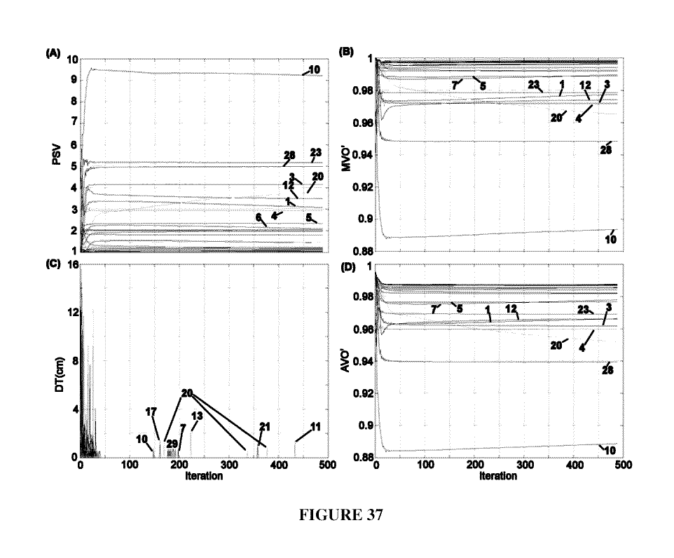

The qualitative evaluation may comprise plotting the values of PSV, MVO, MOV′, AVO, AVO′, and DT or a subset of these with respect to each successive iteration of the data mining algorithm. Example convergence curves derived from synthetic data are given in FIGS. 30, 32, and 33. Convergence curves derived from real EEG data are given in FIGS. 36 and 37. An evaluation may be made by determining which components have volume-domain characteristics that improve and then converge to a final value over successive iterations in the data mining process.

FIG. 30 shows a number of convergence curves illustrating convergence characteristics of the peak spectral value (PSV) over iterations calculated from synthetic EEG data; (A) for each component at each iteration; (B) for the average and median calculated across components for each iteration. Components identified by ICA are labelled (1) through (8). The horizontal and vertical axes are log-log scale to emphasise early changes in the PSV. The vertical axes are unitless. The volumetric spectrum from which the PSV is derived is calculated using a ratio of variances.

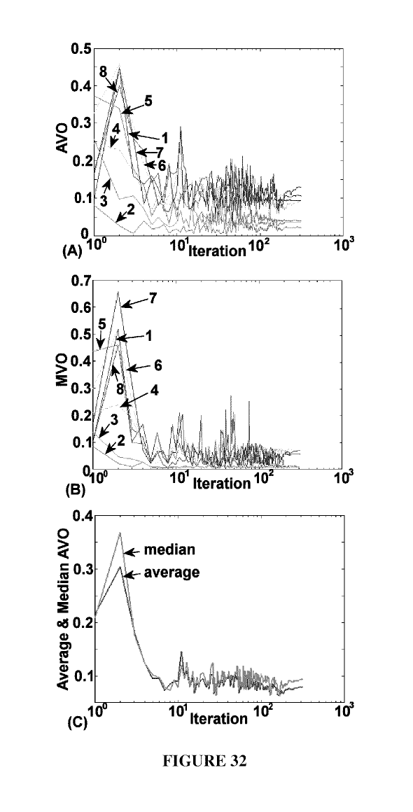

FIG. 32 shows a number of convergence curves illustrating convergence characteristics of the average volume overlap (AVO) and the median volume overlap (MVO) over iterations calculated from synthetic EEG data; (A) the AVO for each component; (B) the MVO for each component; (C) for the average and median AVO calculated across components for each iteration. Components identified by ICA are labelled (1) through (8). Plots are log-log scale to emphasise early changes in the MVO and AVO. The vertical axes are unitless as values are derived as dot products of volumetric spectra.

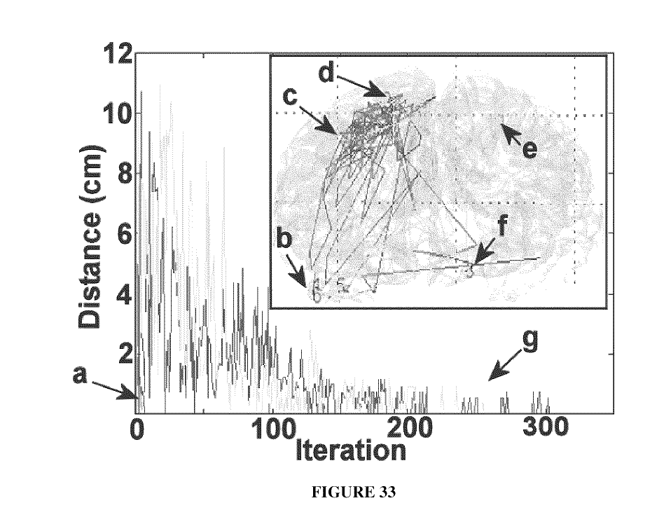

FIG. 33 shows a number of convergence curves illustrating movement of component centers of mass for each iteration of the runica maximization of statistical independence revealing major differences between components of source activities versus components resulting from rank estimation error (calculated from synthetic EEG data). Main Figure: Distance travelled (DT) is the difference in location of centres of mass from one iteration to the next. The distances travelled for the rank estimation error components (7) and (8) are large and carry through past iteration 200. These are given in light-grey (component 7) and dark-grey (component 8). The late center of mass changes for these components is indicated by label (g). The source activity components (1, 2, 3, 4, 5, and 6) travel short distances and convergence before iteration 15 as indicated by label (a) and are barely visible on the figure. Inset: A Frontal view of 3D model brain. Path of travel is indicated as lines within the model cortex. Components are labelled as (b) proximal brain activity components (1, 5, and 6); (f) multi-voxel brain activity component (3); (d) central distal brain activity component (2); (e) electrode artefact component (4); (c) web of path of travel indicated by connecting lines for components (7) and (8). Most connecting lines visible in the figure result from movement of (7) and (8) labelled in light-grey and dark-grey, respectively.

FIG. 36 shows a number of convergence curves illustrating convergence characteristics of the average volume overlap (AVO), median volume overlap (MVO) and the peak spectral value (PSV) of components over iterations calculated for real EEG data; (A) the average and median PSV calculated across all components of the decomposition; (B) the average MVO and AVO, and the median MVO and AVO calculated across all components of the decomposition. The vertical axes are unitless as values are (A) derived as a ratio of variances and (B) derived as dot products of volumetric spectra. Average values calculated across components are indicated in dark-grey while median values calculated across components are indicated in light-grey.

FIG. 37 shows a number of convergence curves illustrating convergence characteristics of each component indicated by the (A) peak spectral value (PSV); (B) the median volume overlap (MVO); (C) the total distance travelled by the center of mass in centimetres (TDT); (D) the average volume overlap (AVO). The vertical axes for (A, B, and D) are unitless; the volumetric spectrum from which the PSV is derived is calculated using a ratio of variances and the median and average volume overlaps are dot products of volumetric spectra. In plot (C) those components with late iterations are indicated in the figure. In plots (A, B, and D) only those components with final values that are visually distinguishable have been labelled. (See Table 1 for the final values of all components.)

### Quantitative Validation: Ranking of Final Volume-Domain Characteristics

Quantitative validation of components provides automation for component selection so that the data analysis process from step 5 of the overall analysis methodology through to step 10 can execute without having the user make decisions that could influence the results. Quantitative validation can be done, for example, using the final values of PSV, MVO, MOV′, AVO, AVO′, and TDT or a subset of these.

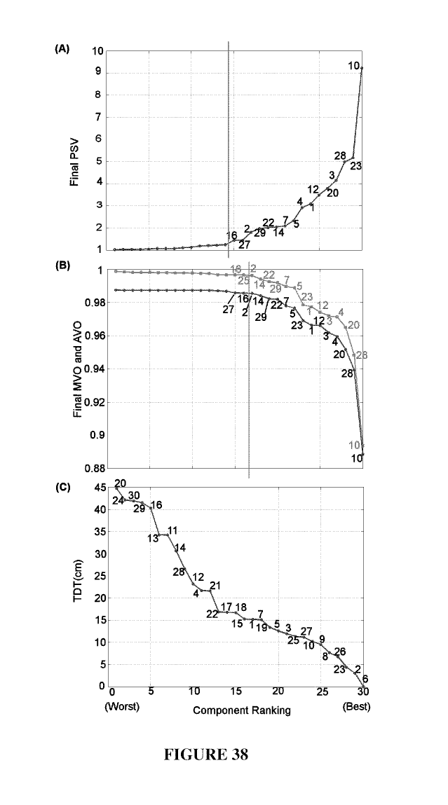

The step of quantitative validation may use the final volume-domain characteristics of each component found in the data mining process to determine which of the components to reject as artefacts. This can be done in a number of ways. One way is to rank the components for each of the volume domain characteristics and then determine a threshold for which components should be rejected. The threshold for each of the volume-domain characteristics can be determined from plots showing the sorted components. This can also be accomplished using an automated process of clustering of volume-domain characteristics where those components that relate to poor estimates of distinct sources cluster together. Component ranking curves and selection of components that are good representations of distinct brain sources from poor representation is illustrated in FIG. 34 for synthetic data and in FIG. 38 for real EEG data.

FIG. 34 shows ranking results for components calculated from synthetic EEG data. (A) peak spectral value (PSV); (B) total distance travelled (TDT); (C) median volume overlap (MVO); (D) average volume overlap (AVO). Component numbers are indicated on the plots next to each rank position point (+). Wherever possible, a vertical line was placed in each figure where natural features in the data separate artefacts from brain activities. The plots shows that those components that score well on all three measures of MVO, PSV, and TDT are ‘good’ modular brain sources.

FIG. 38 shows ranking results for components calculated from real EEG data. (A) peak spectral value (PSV); (B) median and average volume overlap (MVO and AVO); (C) total distance travelled (TDT). Component numbers are indicated on the plots next to each rank position point. A vertical line was placed in (A) and (B) to indicate the knee of the curve. (No knee is evident for (C).)

In each of FIGS. 34 and 38, a horizontal or vertical line drawn through the curves indicates the threshold selected. Those components that have all of the volume-domain characteristics on the distinct modular source side of the threshold are retained while those that do not are rejected.

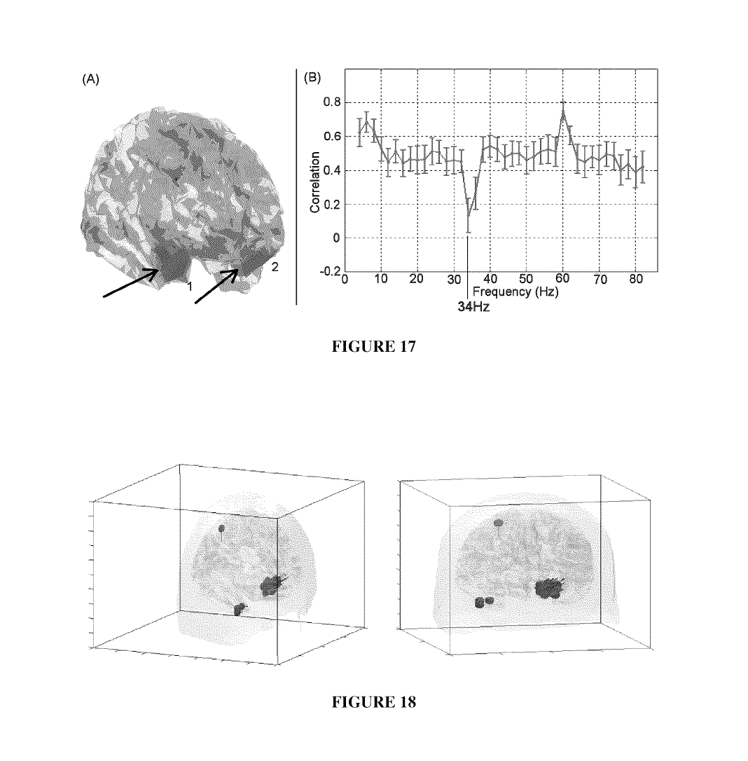

The quantitative validation and qualitative evaluation have one important caveat. That is, if the mathematical head model used in the volume-domain calculations includes the ability to resolve sources located between the skin and the skull, then there is the possibility that components which pertain to specific muscles or the eyes can be validated as good distinct modular sources. This however is not a problem, because these are canonical sources and can be easily detected against a template and can be removed from the group of sources originating from the brain. For example, FIG. 17 illustrates a case where artefacts relating to the eyes are well-represented as sources within the head and are accurately depicted.

FIG. 17 shows results of components generated by a shaped decomposition. (A) The volumes of components 1 and 2 for 5 standard deviations above the volume noise estimate. The new component volumes are the darkened regions beneath the orbitofrontal cortex indicated by arrows. These new component volumes are positioned in the location of the eyes. (B) The pair-wise correlation as a function of frequency for components (1 and 2). Error bars are the standard deviation of random samples of the mean.

In another embodiment, numeric values of one or more of the PSV, AVO, AVO′, MVO, MVO′ and TDT are compared against a template. This comparison is used to determine which components are artefact and which components are good representations of brain activity origination from an anatomically modular region of the brain. These values are compared against a database to determine what an acceptable threshold is to classify a component as artefact or modular brain source.

In another embodiment, this method of volume-domain validation could be combined with a method of characterizing and validating components by their time-varying waveform properties. In such a case, the frequency spectra of the time-varying waveforms of each component, or some other transformation could be used in the validation process.

STEP 10: Generation of Numeric Output with Respect to Task Software Events and Participant Response Events

In an example embodiment, for each participant dataset, after it has been processed in steps 1 (referencing), 2 (artefact rejection), and 3 (mapping data to canonical sensor positions for the purpose of improving comparisons between participants), the source waveforms of each validated component are calculated by multiplying them by the sphering matrix and weight matrix calculated in the data mining process.

These matrices are multiplied as in Equation 8 where ŝ represents the component activities of the participant's dataset. Before multiplication of the participant's data by the sphering matrix and weight matrix, the mean of each sensor channel is subtracted to yield x. The time-varying activities of each source is contained in the columns of ŝ.

In some applications it is required that the ‘RMS-projected’ source amplitude be determined and used in subsequent calculations. This may be calculated by projecting the source waveform to the scalp domain through the mixing matrix. Calculation of the mixing matrix Â is given in Equation 9.

To do the projection of a single component to a topographical equivalent, the matrix ŝⱼ is created where all elements of ŝⱼ are set to zero except for the row j containing the source activation waveform of the component of interest. This yields the scalp-projected component waveform, x′ⱼ as in Equation 27.

x′ⱼ=Âŝⱼ (27)

Calculating the ‘RMS-projected’ value may be accomplished by calculating the RMS value across the sensor dimension (the columns) of x′ for each time sample separately. This yields x′ⱼₘₛ. For the purpose of generalizing in subsequent description, we will simply refer to the source time-varying activities of each component as the component waveform.

The EEG or MEG data may be analyzed with respect to the task that the participant was engaged in when the data were collected. This could be either a Complex Task (where events could be trial starts, or button presses for example) or a Simple Task (where the event is the start of recording data while eyes are closed, start of recording while eyes are open). If for example, eyes-open data are recorded and the participant experiences brain seizure activity, then an event of interest to examine would be the onset and continuation of the seizure.

For a given event that is either generated by the task software or is of a participant response during the task, the EEG or MEG data could be analyzed for an interval before the event, during the event, or after the event. For example, analysis for an interval before the event might be done to examine brain function related to planning for the event. Analysis could also be done on the instantaneous samples of data.

### Analysis of Intervals of Data

Given an interval for analysis identified according to an event in the task software (for example, the start of a trial or the presentation of a reward to the participant) or a participant response event (for example, a button press), the EEG or MEG data can be analyzed with respect to the event in the following way.

These steps can be repeated for each participant and experimental condition in each task and are repeated for each component yielded by the data mining algorithm that has been validated according its volume-domain characteristics. Example methods of calculating activation and coordination, determining the significance of coordination, comparing experimental conditions, comparing a participant against a group, and examining group consistency and outlier identification are given.

### Activation: Root-Mean-Square (RMS)

In some cases, it is the activation of brain areas that is of interest by the user or required in the application in which the disclosed technology is used. The steps below could be followed to calculate activation.
1. Select a segment of EEG or MEG data for analysis corresponding to the event of interest.
   a. The length of this segment can be anywhere from 2 samples of data to a larger user-defined-number of samples.
   b. The segment of data could filtered to emphasise or de-emphasis particular frequency bands.
   c. The segment of data could be multiplied by a windowing function (for example, a Hanning window or a Hamming window) for the purpose of deemphasising the start and end of the segment of data.
2. Each sample contained in the segment of data is then squared.
3. The mean of the squared samples is calculated across segment.
4. The square-root of the mean is calculated.

The result is the RMS activation for the segment of EEG or MEG data with respect to an event in the task software or a participant response event

### Coordination: Correlation, Coherence, Mutual Information

In some cases, an estimate of the coordination among components is of interest. Coordination may indicate that specific modular areas of the brain represented by different components are communicating with one another. Coordination can be estimated in a number of ways. For example, one may calculate: correlation, coherence, or estimates of mutual information, magnitude squared coherence, minimum description length coding, phase locking value, synchronization likelihood, and dynamic itinerancy with synchronization entropy, or some other method. In each case, the estimate of coordination is calculated from the time-varying waveform activities of the validated components.

The estimate of coordination can also be done for multiple pair-wise lags. For example, the lagged-coordination of two different components can be estimated using each of the coordination estimates above. However, if a lagged measure of coordination is of interest, then for each pair of components examined, the interval of analysis of one component will differ by a selected lag (time shift) with respect to the other component's interval of analysis.

Using the calculation of correlation as an estimate of coordination, the following steps can be used for the activities of all validated components. Similar steps could be used for both zero-lagged and non-zero lagged coordination estimates.
1. Select a segment of EEG or MEG data for analysis according to the event of interest for each component pair.
   a. The length of this segment can be anywhere from 2 samples of data to a larger user-defined-number of samples.
   b. The segment of data could filtered to emphasise or de-emphasis particular frequency bands.
   c. The segment of data may be multiplied by a windowing function (for example, a Hanning window or a Hamming window) for the purpose of deemphasising the start and end of the segment of data.
2. Calculate the value of correlation between the pair of segments.
   a. The calculated value can be transformed by an absolute-value function to transform all negative-valued functions to have positive values.
   b. The calculated value of correlation can be transformed using a mathematical function or empirically determined curve for the purpose of mapping the distribution of correlation value to a Gaussian distribution. For example, this function could be atanh.
3. Repeat steps 1 to 2 for all other pairs of components that have been validated.

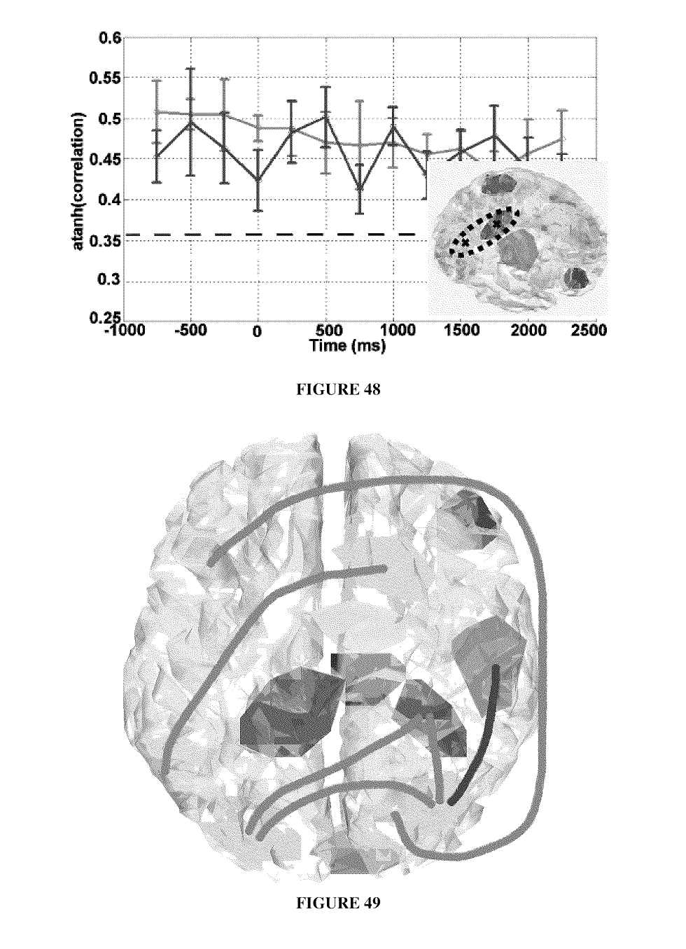

The result is an estimate of the coordination between all pairs of components for the interval of time selected for analysis with respect to an event in the task software or a participant response event. FIGS. 41 and 48 illustrate examples where correlation was used to estimate coordination.

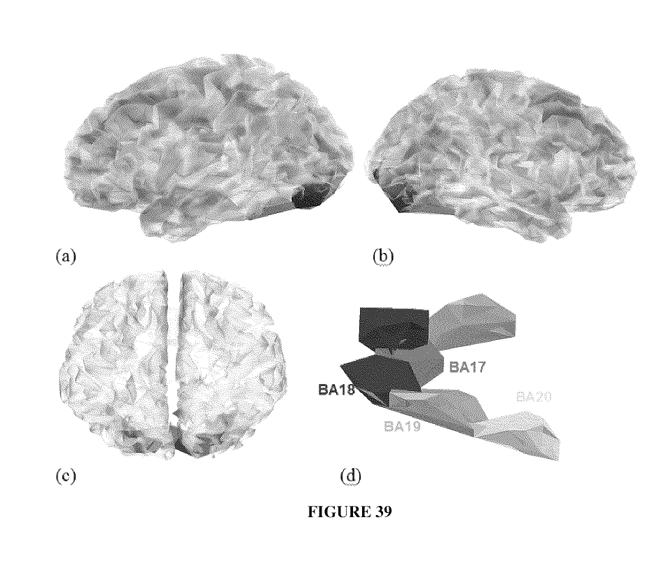

FIG. 39 shows example active modular brain volumes depicted on a white matter head model calculated from EEG data from a place/cue dataset using the disclosed technology. These data were collected from a single subject recording session. Volume edges are defined at 4 standard deviations above the mean noise level (STDM) originating in each ICA topography projected into the head model volume. Two lateral perspectives (a and b) and a posterior view (c) are provided. View (d) is a close-up of active regions of the dorsal visual pathways from (a, b, c). Shades of grey used indicate symmetry across the midline. The brain model grid spacing is 0.5 cm.

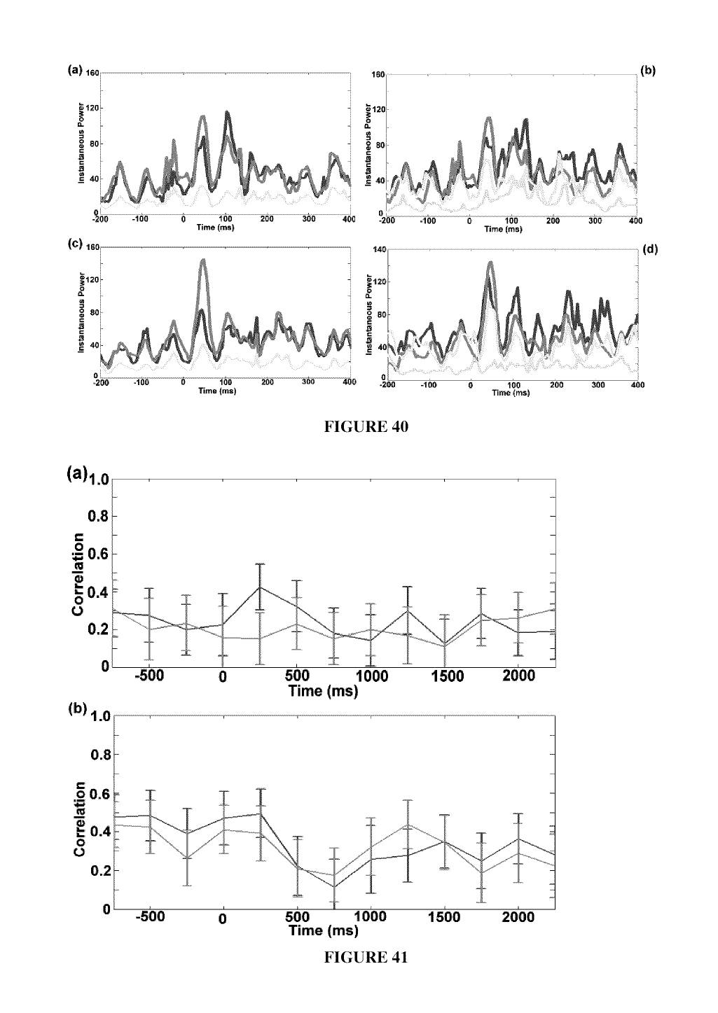

FIG. 40 shows an ensemble averaged instantaneous power as a function of time for activities of the brain volumes of FIG. 39 band pass filtered 3 to 40 Hz. Plots (a,c) are left hemisphere and (b,d) are right hemisphere. Plots (a,b) correspond to the cue condition. Plots (c,d) correspond to the place condition. The shades of grey of the lines in this figure match the volume shades of grey in the volume estimation illustrations. Instantaneous power was calculated as the square of each time sample for each individual trial. The power activities calculated for each individual trial were then ensemble averaged to obtain the current figures. This shows the stimulus locked power fluctuation of multiple visual areas of the brain in relation to the start of the trial.

FIG. 41 shows two examples of the time-varying pair-wise zero-lag correlations in the frequency band 34 to 36 Hz relating to a subset of the brain volumes illustrated in FIG. 39 (Blue=cue condition, Red=place condition). (a) The case that there is a difference in correlation between two behavioural conditions. There is a difference between cue and place for the interval around 250 ms for correlation between hand area (H) dorsolateral region of the left hemisphere frontal lobe and Brodmann area 18 of the right hemisphere. (b) The case that there is a large change in the time-domain correlation between two brain areas over time. There is a difference between the windows centered at 250 ms and 500 ms for both the cue and place conditions. The correlation for this case was calculated between the two ventral Brodmann areas 18 of the left and right hemispheres. Pair-wise correlation is measured in intervals of time, using 500 ms windows with 50% overlap. Plotted points in the figure indicate the center of each window. Error bars represent the standard deviation of the mean of 2000 random samples of 20 trials from the trials of each condition.

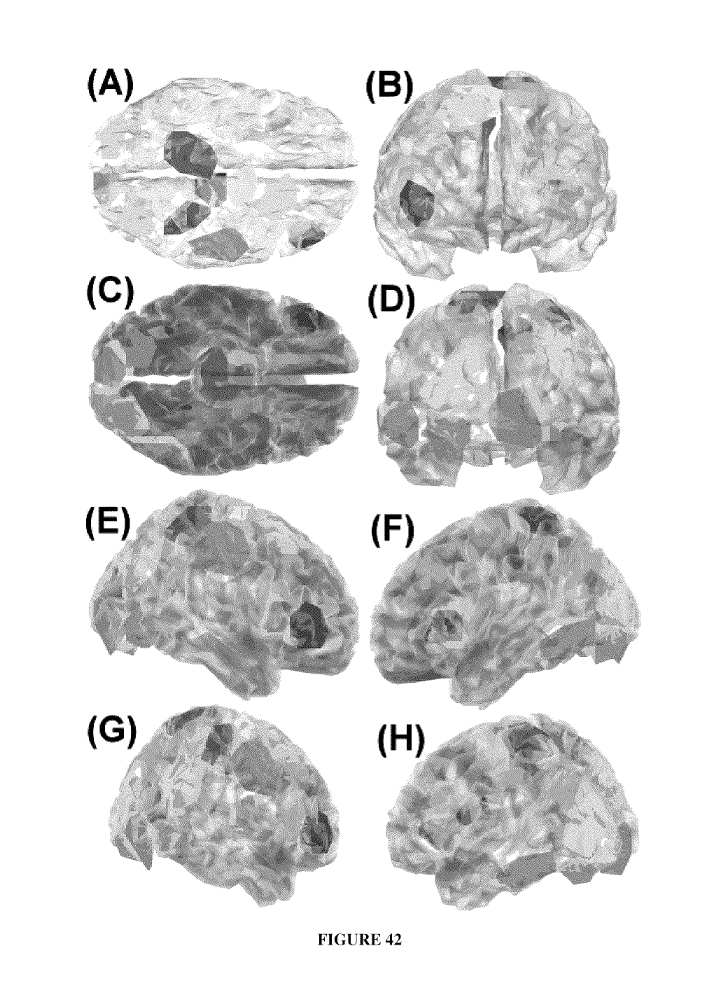

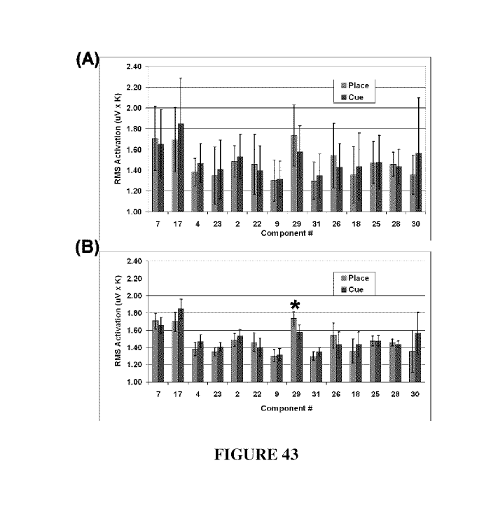

FIG. 42 shows volume regions estimated for component source origins calculated from the EEG data of the participant group illustrated on a canonical cortex. Areas colored as various shades of grey represent the estimated source volumes pertaining to activities of the brain for 5 standard deviations above the mean (STDM) volume-domain noise estimate.

FIG. 43 shows comparisons of RMS activation levels for the first second of spatial navigation for each component between the cue and place conditions. Error bars are the 95% confidence interval. (A) activation for non-standardized data; (B) activation for standardized data. A significant effect of condition is present for component 29 for standardized data. Significant differences are indicated by (*).

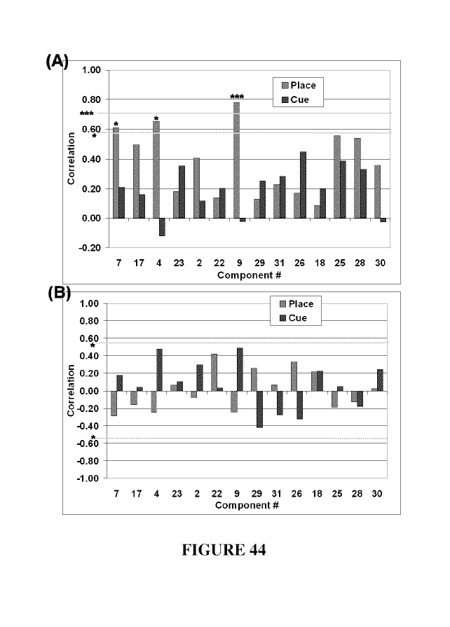

FIG. 44 shows correlation of 8-30 Hz RMS brain activations with behavioural measures. (A) Correlation of behavioural trial completion latencies with RMS activations of each component across study participants. (B) Correlation of RMS brain activations with DS measures of explicit knowledge of platform location. Significant correlation (p<0.05; r>0.576) is indicated by (*) in the figure. (***) indicates significant correlation (p<0.01; r=0.708).

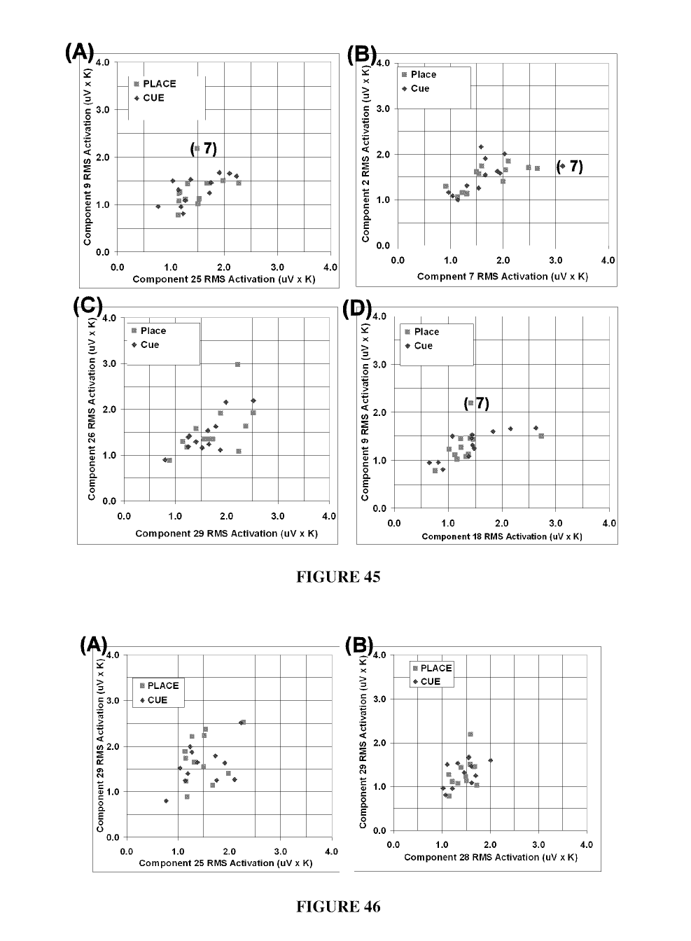

FIG. 45 shows scatter plots depicting RMS activations of a selection of paired components for the first second of spatial navigation demonstrating components that are correlated across study participants for one of the behavioural conditions. (A) relationship of components 9 and 25 is significant for cue but not for place; (B) relationship of components 2 and 7 is significant for cue and for place; (C) relationship of components 26 and 29 is significant for cue but not for place; (D) relationship of components 9 and 18 is significant for cue but not for place. Plots (A, B, D) illustrate a possible outlier (participant 7, indicated in the plots) that has been included in these significance calculations and has not been rejected because it is difficult to determine if this participant is a true outlier given the small sample size.

FIG. 46 shows scatter plots depicting RMS activations of a selection of paired components for the first second of spatial navigation demonstrating components that are not correlated across study participants for both the cue and place conditions. (A) components 29 and 25; (B) components 29 and 28.

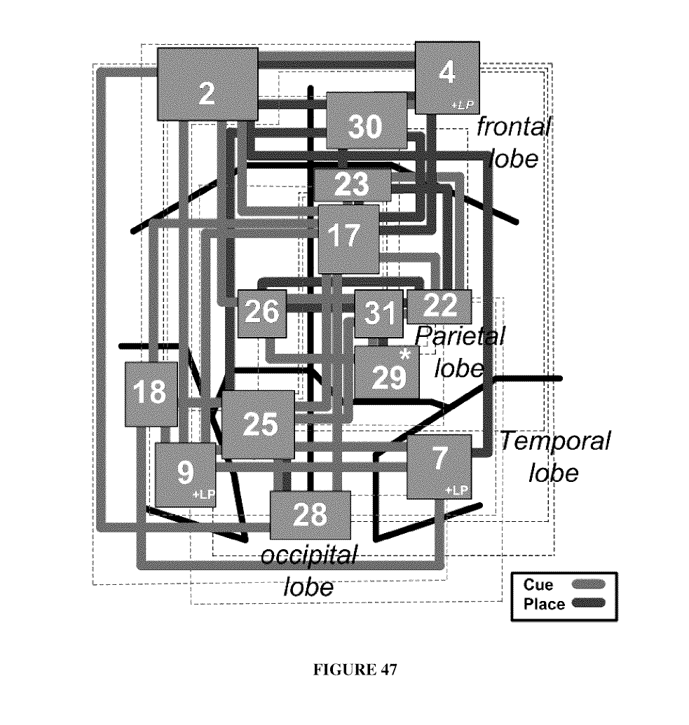

FIG. 47 is a block diagram of pair-wise correlation relationships calculated across the participant group (including possible outlier participant number 7) for the cue and place conditions. Relationships were calculated using the non-standardized RMS activations for the first 1 second of cue and place behavioural trials. Significance levels for this figure provide thresholds by which relationships between components are represented. Thick lines correspond to correlation greater than 0.71 (p<0.01), while thin dashed lines correspond to correlation greater than 0.66 and less than 0.71 (p<0.02). Only lines indicating relationships greater than p<0.02 are given so that only primary relationships are represented. Block numbers correspond to component numbers. Blocks have been spatially arranged in the figure to represent their relative locations in a dorsal view of the cortex. Black lines represent divisions of the cortex: occipital, temporal, parietal, and frontal for each hemisphere. The primary visual cortex (component 28) is represented at the bottom of the figure as a single block. (The primary visual striate cortex was not split into a left hemisphere component and a right hemisphere component by data mining.) Filled colors of blocks correspond to the colors of the estimated volumes in FIG. 2. Blocks have been annotated to show additional information. (*) indicates the RMS activation is significantly greater in the place condition than in the cue condition. LP indicates activity in this area has a correlation relation to place latency measured across the participant group. (+/−) indicates positive and negative correlation, respectively. Italic LP indicates an approach to significance (0.576>|R|>0.4) while non-italicized annotation indicates a significant correlative relation (|IR|≧0.576; p<0.05).

FIG. 48 shows zero-lag correlation between components 29 (posterior parietal cortex) and 31 (superior parietal lobule) estimating coordination among brain areas (8-30 Hz). Main Figure: Cue condition is indicated in blue. Place condition is indicated by light-grey lines. Horizontal dashed line indicates the (p<0.05) significant correlation level. Error bars indicate 1 standard deviation of random samples of the mean. Figure Inset: Isometric view of posterior right hemisphere of the head model showing the location of components 29 and 31. The region of components 29 and 31 in the head model are highlighted using an ‘x-mark’ at each brain regions and a dashed ellipse encompassing both of them.

FIG. 49. Coordination among brain areas measured via zero-lag correlation estimates for the interval around trial onset, −500 ms to 500 ms, 8-30 Hz instantaneous power. Light-grey lines indicate component pairs having significantly greater correlation in the place condition than in the cue condition. Dark-grey lines indicate component pairs in the cue condition with significantly greater correlation than in the place condition.

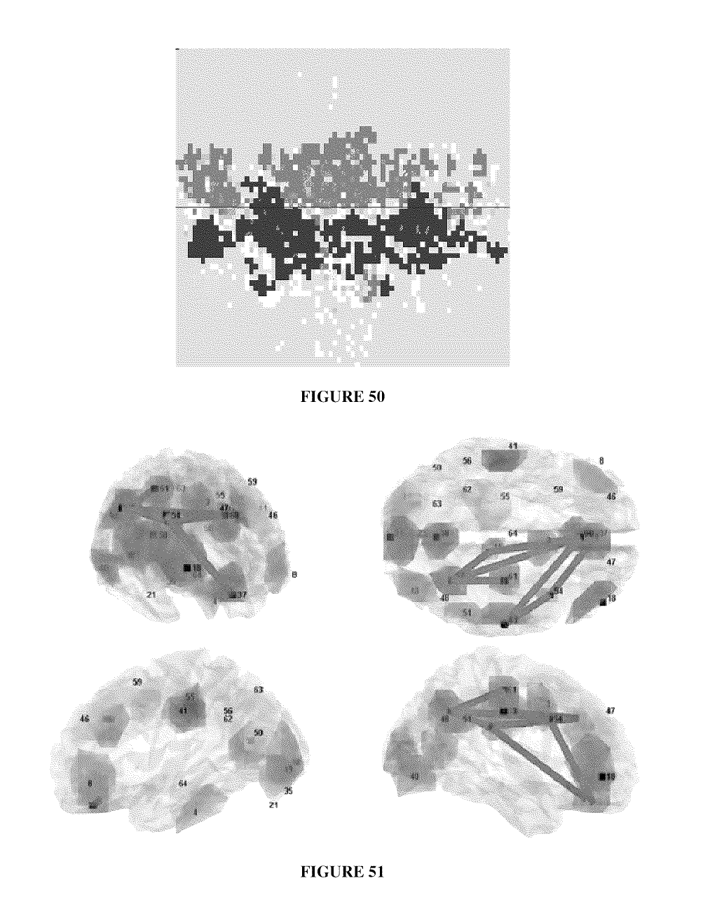

FIG. 50. Heat maps of showing that eye-gaze position on the display screen differs between the ‘cue’ and ‘place’ conditions for the first second of navigation using the vMWT paradigm. Horizontal line indicates horizon that separates visual stimuli in this task that elicit either egocentric or allocentric navigation strategy. Light-grey indicates where participants look in the ‘place’ task that is related to allocentric strategy while dark-grey indicates where participants look in the ‘cue’ task that is related to the egocentric strategy.

### Determining Significance of Coordination

It is often useful to determine if a coordination estimate is significant and that the measured estimate of coordination does not arise out of the natural characteristics of the noise present. This can be done multiple ways. One way is to use surrogate data methods whereby synthetic data are generated either using random samples of the component activity waveforms themselves or by generating synthetic data based on parameters of the component waveforms. FIG. 48 illustrates comparisons of coordination estimates to an established level of ‘chance’ coordination.

A statistical test evaluating the difference between coordination estimates arising from the real data and coordination estimates arising from the surrogate data can reveal if the coordination estimated from the real data simply arise as a property of the noise characteristic of the data.

### Comparing Experimental Conditions

Comparisons of experimental conditions can be made multiple ways. These comparisons can be made with respect to task software or participant behavioural events using outputs such as the RMS activation values, or measures of coordination. Three example methods of measuring the difference between conditions are: subtractive, parametric, and non-parametric. Some parametric methods that can be used are the t-test, ANOVA, or others. A non-parametric test that can be used is the permutation test or random sampling. FIGS. 51 and 54 illustrate comparisons made between two task conditions using non-parametric methods revealing systems of the brain that have greater activation and coordination for one task but not the other.

FIG. 51 illustrates a data-driven model of brain function created from data collected from persons while they navigated a computer-based virtual environment design to elicit cognitive spatial navigation strategies. The difference between two navigation conditions is illustrated showing which regions of the brain are more active during the ‘cue’ maze (designed to elicit brain function associated with simple navigation) indicated by dark-grey regions. Dark-grey lines connecting brain regions indicate the level of coordination between these regions. Those regions marked with a black square indicate brain activity (measure as RMS activity) that is significantly greater in the ‘cue’ condition versus a baseline ‘guidance’ condition. The figure shows activation of multiple low-level (sensory) and high-level areas of the brain and the ways in which they are coordinated with each other. Activity was much stronger and more co-ordinated on the right side of the brain while the participants were navigating in the cue condition compared to the guidance condition.

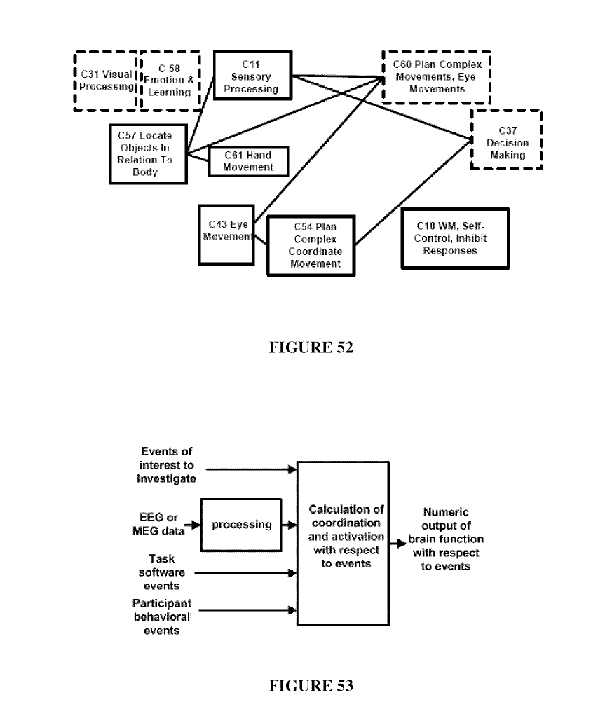

FIG. 52 shows the results of FIG. 51 mapped into a schematic diagram format where the areas of activation above baseline in the cue navigation task are indicated as blocks in the diagram and the relationships of coordination are indicated by lines connecting the blocks. Blocks with solid borders were significantly more active than baseline while blocks with dashed borders were not. (Note: it makes sense that the block labelled C31 relating to activity of the visual cortices was not more active in either the baseline or the cue-spatial condition because both the baseline and the cue-spatial condition required visual processing.) The functional operations of each block are provided from a neuroscience database relating known brain anatomical locations to ‘type of information processing’. This schematic diagram illustrates the relationship of information process and can be used to better understand healthy brain function and disease or dysfunctional brain function. It can also be used to help understand how persons with a brain injury are compensating for their injuries.

FIG. 54 illustrates a data-driven model of brain function created from data collected from persons while they navigated a computer-based virtual environment design to elicit cognitive spatial navigation strategies. The difference between two navigation conditions is illustrated showing which regions of the brain are more active during the ‘place’ maze (designed to elicit brain function associated with complex navigation) indicated by dark-grey regions. Dark-grey lines connecting brain regions indicate the level of coordination between these regions. Those regions marked with a black square indicate brain activity (measure as RMS activity) that is significantly greater in the ‘place’ condition versus a simple-spatial navigation baseline ‘cue’ condition. This is a very important finding in our research; these results obtained using the disclosed technology implicate the coordination and activation of these areas as a possible system of brain function supporting a type of navigation that is impaired in persons with traumatic brain injury (TBI).

### Comparing the Individual to the Group

One way construct a data-driven model of brain function from the data of an individual person is to process it using the disclosed technology as illustrated in the configuration of FIG. 54. Alternatively, if the goal is to compare the data of an individual with respect to a group, then additional options are available. The data of the individual could be concatenated to the group data and the concatenated data could be processed as illustrated in FIG. 54. Another way is to process the data collected from the individual using an a priori defined weight matrix and sphering matrix. In such as case, comparisons of the data from the new participant could be made to the group using a simplified calculation. Data from a group or an individual could be processed, for example, as shown in FIG. 9 for the case that a canonical shaping filter is known.

An individual can be compared to the group in terms of coordination and activation in a number of ways. An intuitive method is using a scatter plot where the processed results of each participant can be seen. Alternatively, automated classifiers can be used to identify subgroups within the main group or identify outliers. FIGS. 4, 45 and 46 illustrate examples of scatter plots used to show how participants cluster.

FIG. 4 is a simplified simulated example of brain function data collected from two persons (triangle and square) who wish to have their brain function assessed on a regular basis with respect to brain function data corresponding to persons with Parkinson's Dementia (X) and Alzheimer's Dementia (O). The figure illustrates that one person (square) having regular assessment has a trajectory of changing brain function heading towards the Alzheimer cluster of samples. In the case of the other person (triangle), there is no conclusive trajectory. The vertical and horizontal axis each represent activation of a different component of brain function related to the behavioural task during which data were recorded identified using the disclosed technology.

### Calculation of Consistency Across the Group and Identification of Outliers and Subgroups

To show the consistency of RMS activation of pairs of components for the purpose of demonstrating differences between two experimental conditions, the correlation across the group for each pair of components can be calculated. If it is preferred to have all positive correlation values, the absolute value of this correlation can be calculated. FIG. 47 illustrates results obtained in an investigation of spatial navigation behaviour for two types of task: a cue task, and a place task. The consistency of RMS activation of pairs of components calculated across the group for the two conditions is graphically depicted in the figure.

### Part 2: Application Examples Including Associated Peripheral Apparatus

### Standard Equipment Configuration

A typical apparatus configuration for acquiring EEG or MEG data from a person or animal for use with the disclosed technology is provided below, however an alternate equipment configuration could be used.

The apparatus configuration generally comprises:
an EEG and/or MEG machine for collecting data containing brain activity from the participant in an investigation,
a display screen (or output device or another modality such as a loud speaker) for providing the participant with information relevant to the task,
a processor for running software that provides the task in which the participant will participate and for synchronizing data. For example:
   EEG or MEG data as it arrives from the EEG or MEG machine,
   Data containing events in the task software (such as moments of stimulus change or moments of task trial start and end),
   Data containing the responses of the participant (such as is identified by movement of a joystick or other input device),
   Data acquired from any other behavioural or biometric measurement device such as an eye-tracker, or heart-rate monitor, or galvanic skin-response measurement device,
an input device to the processor (such as a joystick) so that the participant can interact with the task software running on the processor,
a disk for recording data from the EEG or MEG equipment, data containing events in the task software, data containing participant responses, and any other types of data of interest.

FIG. 1 and FIG. 2 illustrate an example of how the apparatus may be interconnected with respect to the subject for collecting data that can be processed using the disclosed technology. Depending on task and paradigm requirements, the participant could be awake, sleeping, or in a non-communicative or “locked-in” state, such as being in a coma or having a sever disease that interferes with standard methods of communication.

In the case that EEG data are collected, EEG electrodes can be placed uniformly or non-uniformly on the subject's scalp. The disclosed technology can compensate for non-uniform electrode spacing and compensate for spatial gaps in electrode placement. Similarly, the disclosed technology can compensate for spatial gaps in the MEG sensor array.

FIG. 3 depicts the equipment configuration and placement of a participant engaged in a computer-based task. The task shown in the figure was used to elicit the activities of particular brain systems so that their activities could be recorded in relation to participant responses to the task and in relation to events in the task software. Brain function analysis results pertaining to this task are given elsewhere in this document.

### Typical Data Collection Paradigm

This standard data collection paradigm is referred to by multiple application examples in this document and is hence outlined here for brevity.

The disclosed technology can be used to process data from two types of tasks. The first type of task is called a ‘complex task’ in which the participant interacts with the task software. A complex task is a task designed to elicit complex brain function. For example, the participant might be pressing buttons or navigating a maze. To support this behaviour, the participant's brain will have multiple areas or systems coordinating to facilitate the button pressing or navigating behaviour. The second type of task is a ‘simple task’ in which the participant does not interact with the task software.

### Example Complex Task

A complex task could be a game of card sorting, object naming, or navigating in a computer-based virtual environment. For example, the disclosed technology has been used in conjunction with a task called the virtual Morris Water Task (vMWT) designed to elicit various types of spatial navigation behaviour and non-spatial navigation behaviour. In eliciting types of spatial navigation behaviour, the task elicits spatial navigation cognition and the underlying activities of systems of the brain associated with these types of cognition. Similarly, components of the task that elicit non-spatial navigation behaviour elicit non-spatial types of navigation cognition and the brain function associated with this type of cognition. In our own investigations, we have used the disclosed technology to identify the systems of the brain associated with allocentric and egocentric types of navigation, and some instances, have demonstrated, when these different systems are active with respect the events in the navigation task paradigm. Results of these investigations are provided in this document.

A complex task could also be comprised of a battery of one or more neuropsychological tests or more generally, any set of activities from which psychometric data can be obtained. This could be embodied as a game that is either computer-based or non-computer based. Tests designed for neuropsychological testing, while providing the ability to assess brain function, also cause activation of those areas being assessed. Hence, this activation could be recorded using EEG methods and then processed using the disclosed technology to reveal the activities of systems of the brain involved in the task of doing these tests.

A complex task could also be a neurofeedback task where the participant receives stimulus from a device in response to the measured activities of their brain real-time or pseudo-real-time. Control of a wheelchair, prosthetic limb, computer or the like may be implemented in response to measures of the levels of one or more sources.

A complex task could also be embodied as a game created purely for the purpose of entertainment of the person playing the game. Other complex tasks could be: mentally rotating an object, learning a foreign language.

### Example Simple Task

An example simple task is often referred to as “eyes-closed/eyes-open”. In this task, an interval of data is collected while the subject has their eyes either open or their eyes closed or for both eyes-open and eyes-closed. In such a task, mental imagery could also be a part of this task.

FIG. 53 is a high-level block diagram illustrating the processing relationship of the EEG or MEG data and task software events and participant behavioural event and how they may be combined to generate numeric outputs with respect to the events of interest.

### APPLICATION EXAMPLE 1

### Participant Screening and Homogenous Group Identification by Examining Brain Systems Activation for the Purpose of Minimizing Group Variance in CNS Pharmacological Investigations

In investigations of CNS pharmaceutical therapies and treatments it is beneficial to minimize between-participant variability. Doing so can help reveal a drug effect that would be otherwise hidden by differences between participants. A screening process that uses brain function characteristics as screening criteria alone or in combination with other criteria has the following advantages:
1. Creates homogeneous groups having similar brain function.
2. Supports the use of characteristics of brain function as evaluation criteria for determining if a drug is appropriately affecting target areas of the brain.
3. Provides for the opportunity to create subgroups from the participant pool that have particular disease characteristics

Using standard behavioural or psychometric methods, classification of disease stage of progression or even the type of disease present can be difficult, particularly at the early stages of a disease. For example, diseases such as Parkinson's disease/dementia and Alzheimer's disease/dementia are typically grossly classified as early-stage, mid-stage, and late-stage according to behavioural symptoms and psychometric scoring and potentially miss important aspects of the continuous pathology of the disease. There are often multiple interfering factors that make classification difficult or cause classification errors. In contrast, classification according to brain function would potentially identify more characteristic stages of the disease and a sensitive metric that can then be used as a dependent variable in the drug investigation.

The disclosed technology can be used in the participant screening process in investigations of drug therapies and treatments for a variety of brain diseases, brain dysfunctions, and brain injuries that include: Parkinson's disease, Alzheimer's disease, Parkinson's dementia, Alzheimer's dementia, schizophrenia, dyslexia, attention deficit hyperactivity disorder, mild cognitive impairment, traumatic brain injury, seizure disorders, and stroke. It can be used to classify potential participants for an investigation of therapies or treatments for these diseases.

Below are example steps that can be followed for using the disclosed technology for participant screening for a drug study.
1. Position the participant and connect equipment according the description in the Standard Equipment Configuration section of this document
2. Begin recording EEG or MEG data
3. Begin the data collection paradigm and have the participant do tasks that are appropriate for the drug that will be investigated and record data containing events in the task software and data containing responses of the participant
   a. The tasks selected could have 3 components:
      i. Simple Task: eyes-open/eyes-closed
      ii. Complex Task: activate systems of the brain that the drug is designed to affect, and
      iii. Complex Task: activate systems of the brain that the drug is not designed to affect
4. Upon completion of the paradigm, stop all recording and store the data for later analysis
5. Repeat steps 1 to 4 until all participants have contributed data
6. Use the disclosed technology to process the recorded MEG or EEG data for all participants with respect to the recorded events in the task software and the data containing participant responses
7. Use the numeric output calculated and stored for each participant to create scatter plots and from these scatter plots visually identify outlier participants or subgroups within the participant data. (Alternatively, use an algorithm such as the kmeans algorithm for identifying outliers and subgroups within the participant data.)
8. Using the results of the grouping and outlier identification process do one or both of the following:
   a. reject the outlier participants that differ from the main group and use the remaining participants in the pending drug investigation, or
   b. examine the brain systems active for each subgroup and select the participants in the subgroup with the brain system activation that best suits the pending drug investigation. For example, if the drug is designed to increase the activity of particular system of the brain, the potential participants that are underutilizing these systems could be selected so that increased activity and use of these systems (caused by use of the drug) can be demonstrated. Conversely, if a drug is designed to reduce the activity of a specific system of the brain, then participants could be selected that have high activity in these systems and then use these participants to show that the drug can reduce their activity. In cases where compensation for a disease or dysfunction is believed to be taking place, participants can be selected based on the activation of compensatory systems for damaged or dysfunctional systems of the brain that the drug therapy or treatment is designed to affect. The goal in this case would be to show that the activities of compensatory systems decrease when the drug is consumed.

### Brain Function Effect Proof, Personalized Dose and Schedule

### APPLICATION EXAMPLE 2

### Determining for Groups of Persons and/or for Individual Persons if a Drug Affects Target Brain Systems and does not Affect Non-Target Brain Systems

It is useful to demonstrate that a drug appropriately affects target brain systems and does not affect non-target brain systems. It is also useful to determine which persons in the population, or which subpopulations respond positively to a particular drug and which persons or subpopulations respond poorly or have negative side-effects to the drug. This can be done after a drug has been accepted for general public use or during the investigational phases of the drug. Doing so allows an investigator to show that the drug performs as intended or a clinician or medical doctor to determine if the drug is suitable for their patient.

Determining efficacy of a drug is essentially to determine that the target brain systems are appropriately affected by the drug. Similarly, to determine if there are brain function side effects is essentially to determine if non-target systems of the brain are affected by the drug adversely or unexpectedly. For example, it is important that a drug treatment or therapy designed to address movement symptoms in Parkinson's disease does affect the activities of the motor systems but does not adversely affect memory systems of the brain.

Below are example steps that can be followed for using the disclosed technology for identifying if a drug affects target systems of the brain and does not affect non-target systems of the brain. These steps should be followed for both on-drug and off-drug conditions. The order of off-drug and on-drug testing should be randomized across participants.

### Off-Drug Condition Data Collection:

1. Position the participant and connect equipment according the description in the Standard Equipment Configuration section of this document
2. Begin recording EEG or MEG data
3. Begin the data collection paradigm and have the participant do tasks that are appropriate for the drug under investigation and record data containing events in the task software and data containing responses of the participant
   a. The tasks selected could have 3 components:
      i. Simple Task: eyes-open/eyes-closed
      ii. Complex Task: activate systems of the brain that the drug is designed to affect, and
      iii. Complex Task: activate systems of the brain that the drug is not designed to affect
4. Upon completion of the paradigm, stop all recording and store the data for later data analysis
5. Repeat steps 1 to 4 until all participants have contributed data

### On-Drug Condition Data Collection:

Followed steps 1 to 4 as above however, this time the participant is receiving the drug treatment or therapy. Store the data collected for later data analysis

### Data Analysis:

To analyze the collected data using the disclosed technology, the steps below can be followed.
1. Use the disclosed technology to process the recorded MEG or EEG data for all participants with respect to the recorded events in the task software and the data containing participant responses and the on-drug off-drug conditions
2. Use the numeric output (for intervals of data related to key moments in the behavioural task) calculated and stored for each participant to create scatter plots and from these scatter plots visually identify outlier participants or subgroups within the participant data. (Alternatively, use an algorithm such as the kmeans algorithm for identifying outliers and subgroups within the participant data.) This can be done for both the on-drug and off-drug conditions separately or it can be done using the subtractive difference between the conditions.
3. from the numeric output:
   a. if the goal is to increase the activity of target brain areas, determine if there is a statistically significant increase from the off-drug state to the on-drug state for target brain areas (for example, a drug to increase dopamine for treating PD should cause an increase in areas of the cortex that receive projections from the striatum)
   b. if the goal is to decrease the activity of target brain areas, determine if there is a statistically significant decrease from the off-drug state to the on-drug state for target brain areas (for example, a drug to decrease dopamine for treating schizophrenia should cause a decrease in areas of the cortex related to uncontrolled thoughts and perceptions)
   c. if the goal is to show that the drug under investigation does not affect specific brain systems, make statistical comparisons of the on-drug and off-drug state for the part of the paradigm where the task is performed that elicits activations of systems of the brain that are not supposed to be affected by the drug.

### APPLICATION EXAMPLE 3

### Measuring Brain Function in Relation to Dose and Dosing Frequency for the Purpose of Determining an Effective Dose and Dosing Frequency to Maximize the Desired Drug Effect on Brain Function while Minimizing Brain Function Side Effects

It is desirable to determining the pharmaco-dynamics associated with a drug designed to treat brain disease or dysfunction for homogeneous subgroups of the population for individual persons. It is desirable because often having too little drug in one's system or too much drug can lead to inadequate treatment or side effects, respectively. Identifying optimal drug dosage and dosage frequency for individuals or homogeneous subgroups of the population can lead to improved treatment or therapy for the disease or dysfunction being targeted by the drug and reduce negative side-effects caused by non-optimal dosages and dosage frequency.

For the example of schizophrenia, too much antipsychotic can decrease dopamine too much resulting in motor rigidity and depression while too little antipsychotic does not address the symptoms of schizophrenia. Similarly for Parkinson's disease, a balance is also required. If someone with PD receives too much levodopa, this will lead to too much dopamine in the brain and hence will lead to dyskinesia and agitation while having too little levodopa will not address the motor function and tremor issues associated with too little dopamine.

The disclosed technology can be used in a system of dosage and dosage frequency finding to match the pharmaco-kinetics of the individual person or a homogeneous subgroup of the population. The ideal parameters for drug dosage and dosage frequency can be determined using the disclosed technology by the drug developer during a drug investigation or it can be determined through an investigation of a patient by their physician. This provides an opportunity for developers and for patients and physician's to quickly identify the ideal dosage characteristics.

Two different but related methods of identifying optimal dosage characteristics are required. The first utilizes an intravenous tube to provide the drug directly to the blood stream or a tube to provide the drug directly to areas of the brain and the drug effect is nearly immediate. The second method follows a dosage characteristic and brain function effect measurement schedule that extends over a period of multiple days. The second case could involve starting with 1 dose per day and measuring its effectiveness. If the desired effect is not achieved, the dosage can be increased and the subsequent effect (both positive and negative effects) can be measured until you reach a steady state of medication and/or the desired effect.

The steps of a first example method that has a more immediate drug effect in response to a change in dosage characteristics is described below and can be used for investigating dosage characteristics in a drug investigation. A subset of these steps would be used to investigate dosage characteristics for an individual. This use of the disclosed technology requires a pseudo-real-time implementation so that data can be processed as they are received.
1. Position the participant and connect equipment according the description in the Standard Equipment Configuration section of this document
2. Connect the participant to an intravenous tube connected to a machine that provides the participant with a specified drug dose at a specified dose frequency.
3. Begin recording (buffering) EEG or MEG data
4. adjust the drug dose and dose frequency parameters
5. Begin the paradigm
6. Have the participant do tasks that are appropriate for the drug investigated and record data containing events in the task software and data containing responses of the participant
   a. The tasks selected could have 2 components: (the required tasks can be embodied as an on-going video game)
      i. Complex Task: activate systems of the brain that the drug is designed to affect, and
      ii. Complex Task: activate systems of the brain that the drug is not designed to affect
7. Use the disclosed technology to process the recorded (or buffered) MEG or EEG data with respect to the recorded events in the task software and the data containing participant responses
8. Using the numerical output and graphical output of the disclosed technology verify levels of activity in systems of the brain targeted by the drug and levels of activity in systems of the brain not targeted by the drug.
9. If the requisite brain function has not been achieved, make the required drug dose and dose frequency parameter adjustments and go back to step 6.
10. End the paradigm

### APPLICATION EXAMPLE 4

### Identification of Brain Function Progression Towards Characteristic Brain Disease or Dysfunction

It is desirable to detect the progression of brain function towards a disease or dysfunctional state as early as possible so that steps can be taken to alter the course of progression of brain function change. Treatments and therapies or lifestyle changes made early can delay the end-stage symptoms of some diseases or mitigate the presence of dysfunctions and this can positively impact quality of life of the persons with the disease and their family.

One can characterise brain function related to Alzheimer's dementia and brain function related to Parkinson's dementia by collecting brain function data from multiple persons at various stages of these diseases, and create a data-driven “map” of disease progression. Given the existence of such a map, the brain function of individual persons can be characterized with respect to disease locations on the map. Taking this a step further, given data collected from the same person over multiple recording sessions spaced months apart, a trajectory of brain function change can be calculated with respect to the disease locations on the map. FIG. 4 illustrates the principles involved for a 2-dimenstional map using the example metrics of RMS activation of components of the EEG or MEG data. In practice however, this map will have many more dimensions.

It is difficult to build classifiers of early stage disease or dysfunction since it is often difficult to determine if persons have an early stage of a disease or dysfunction; there is often compensation taking place and behavioural measures are often indeterminate. This method of determining a trajectory for persons to compare them to disease or dysfunction classes has the advantage that it is not absolutely necessary to build a classifier for early-stages of a disease or dysfunction. This is because this method of identifying a vector of change provides information to show that a particular person's brain function is changing on a path towards the disease or dysfunction. Once the person's brain function begins to diverge from the statistical distribution of their peers, towards the mid-stage or late-stage disease classifier, then one can consider that they are in the early stages of the disease or dysfunction.

The disclosed technology may be used for two purposes in this application. Brain disease and dysfunction classes may be identified to facilitate determination of whether a person's brain function is changing towards any of those classes. Once disease and dysfunction classes have been identified using the disclosed technology and a set of numerical results describing the disease and dysfunction classes have been generated, the data of other persons from the population can be compared to these classes. The broad steps in this application example are: (Part I) identification of system-level brain function characteristics of specific brain-related diseases and dysfunctions, (part II) identification of changing brain function trending towards specific brain-related diseases and dysfunction.

### Part I: Identification of System-Level Brain Function Characteristics of Specific Brain-Related Diseases and Brain-Related Dysfunctions

This example describes characterization of brain function of persons with Parkinson's dementia, however a similar set of steps may be followed for other brain diseases conditions or dysfunctions. Data should be collected while participants are not taking a drug treatment or therapy for their conditions if possible.
1. Position the participant and connect equipment according the description in the Standard Equipment Configuration section of this document
2. Begin recording EEG or MEG data
3. Begin the data collection paradigm and have the participant do tasks that are part of a battery of tests that examine many aspects of brain function and record data containing events in the task software and data containing responses of the participant
   a. The tasks selected could have 2 components:
      i. Simple Task: the standard eyes-open/eyes-closed task
      ii. Complex Task: activate many systems of the brain (one task for example, may be the vMWT)
4. Upon completion of the paradigm, stop all recording and store the data for later data analysis
5. Repeat steps 1 to 4 until all participants have contributed data

### Data Analysis:

To analyze the collected data using the disclosed technology for the purpose of characterizing the brain-related disease or dysfunction of interest, the steps below can be followed.
1. Use the disclosed technology to process the recorded MEG or EEG data for all participants with respect to the recorded events in the task software and the data containing participant responses
2. Use the numeric output (for intervals of data related to key moments in the behavioural task) calculated and stored for each participant to generate a set of values for each participant and add them to the ‘map’. For example, these values could be the RMS power of component activations in the time interval around a particular button press in the task. If we consider a 2-dimenstional example, the RMS power of two components in the time interval can be plotted for each participant. In this case, the resulting scatter plot of data from all participants is the ‘map’.
3. A statistical distribution for each of the numeric outputs of the disclosed technology with respect to the events of the task software and the participant responses is calculated across the participant group. In this example, this statistical distribution characterizes the group of participants with Parkinson's Dementia and this statistical distribution provides a reference point to relate data collected from ostensibly healthy persons.
   Part 11: Identification of Persons with Changing Brain Function Trending Towards Specific Brain-Related Diseases and Brain-Related Dysfunction

Since the objective is to identify the trend of a person's brain function moving in the direction towards having properties of the characteristics of diseased or dysfunctional brain function (using PD as the example), it is necessary to collect data from the person being investigated on multiple occasions to reveal a directional vector of change. On each occasion, the same data collection steps 1 through 4 are followed as was done for the creation of the disease or dysfunction dataset.

FIG. 4 illustrates a 2-demensional example for two different ostensibly healthy persons wishing to have their brain function examined to determine if their brain function is trending towards that of a characterized disease or dysfunction.

### EXAMPLE 5

### Application Software Installed on a Computer or a Client-Internet Server or Cloud-Based System Used by Users Such as Brain Researchers and Quantitative-EEG (QEEG) Practitioners, and Medical Personnel, or Other Persons to Verify that Artefacts Selected for Removal by Automated Artefact Rejection Algorithms do Indeed Physically Originate from Artefact Sources

A problem associated with automated artefact removal algorithms is that the users of the algorithm generally do not trust that the algorithm will only remove artefact from the data and not unintentionally remove some variance related to brain activity. Hence, many potential gains by automated artefact removal algorithms have not been realized. For example, a brain researcher using an automated noise removal algorithm risks having the experimental effect he or she is interested in finding removed from the data. Similarly, medical personnel using automated artefact removal algorithms are often concerned that a feature pertaining to abnormal brain function might be removed from the data and their patient's life might be put at risk because of missed symptoms.

Using the disclosed technology in an application of EEG or MEG artefact removal helps to address this problem. The disclosed technology does so by adding the functionality to an artefact removal process to map artefacts to their equivalent anatomical volumes and allows the user of the algorithm to decided if an artefact that is modeled as, for example, an eye-ball or muscles of the eye, to choose to reject it from the data or keep it in the data.

There are a few simple examples where inclusion of the disclosed technology in artefact removal processes will provide immediate benefit. In the QEEG practices, it is not uncommon to avoid automated artefact rejection algorithms and to manually select segments of EEG data, by manual inspection, that are believed to be without artefact. By providing QEEG practitioners with the ability to identify the physical origin of artefacts identified by automated artefact removal processes, they will be more likely to use such artefact removal processes and be able to spend less time manually searching the EEG datasets of their clients for segments of artefact-free data. The case is similar for medical personnel in hospital applications and for brain researchers investigating effects on brain function.

While the disclosed technology was originally designed to mine EEG or MEG data to identify activities of distinct modular areas of the brain, the algorithm can also be used to identify distinct and anatomically modular artefacts. This property of the disclosed technology is utilized in this application.

The steps for using the disclosed technology as part of a client-server automated artefact rejection process are given below. Similar steps would be used in a cloud-based implementation and a subset of similar steps would be used in a stand-alone application. Herein we refer to the automated artefact rejection algorithm as the ‘cleaning algorithm’ for clarity.

Example steps are as follows:
1. the user of the system sends EEG or MEG data to the server to be cleaned
2. raw multi-channel EEG or MEG data are processed by the cleaning algorithm to yield a ‘cleaned dataset’ on the server
3. the difference between the raw multi-channel data and the ‘cleaned dataset’ output from the cleaning algorithm are subtracted to yield the multi-channel artefact data that were removed by the cleaning algorithm
4. the multi-channel artefact data removed by the cleaning algorithm are then processed by the disclosed technology and a head model that includes multiple layers that mathematically approximate anatomy between the skin and the skull (this can be a standard 3-shell BERG head model)
5. the disclosed technology identifies which artefacts can be mapped into the head model as physically modular sources
6. the server sends the results of the disclosed technology that identifies which artefacts can be mapped into the head model as physically modular sources to the client application so that they can be viewed by the user
7. the user chooses which of these modelled artefacts fit with known canonical artefact sources (such as the eye-balls, eye-muscles, facial muscles, muscles near the ear and jaw) and sends their choices to the server through the client application
8. the activities of those artefacts that are not selected by the user are then added back to the ‘cleaned dataset’ created in step 2.
9. the new cleaned dataset with only the user-selected artefacts removed is provided to the user.

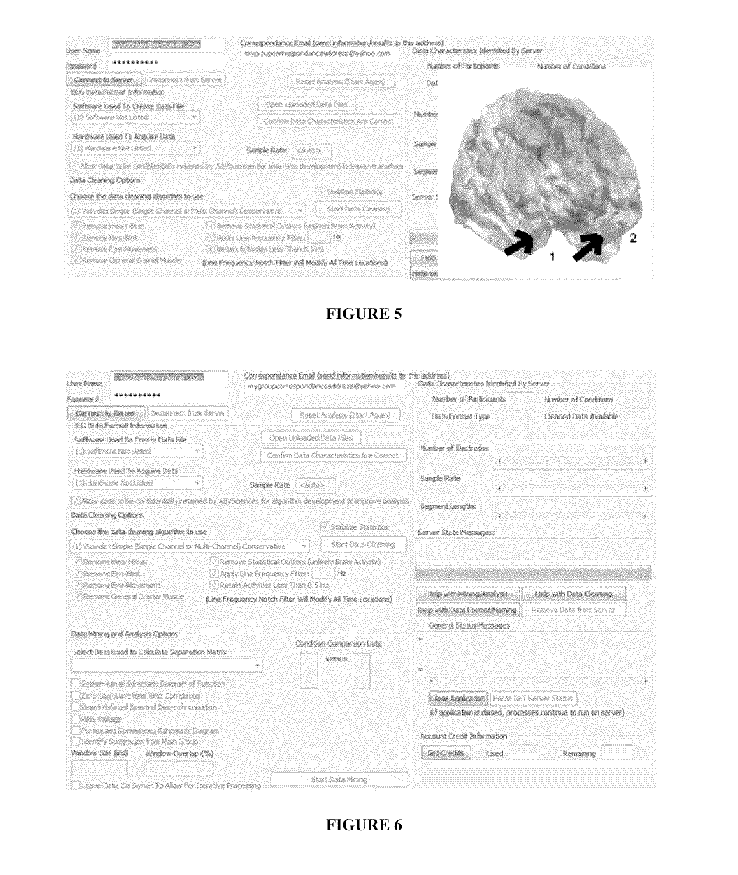

Example use of the disclosed technology in this application is given in FIG. 5 where the client-side user interface is illustrated. In this Figure, the results of the disclosed technology are also illustrated showing that the disclosed technology can identify eye-artefacts from EEG data. Similarly, the disclosed technology has been used to identify muscle artefacts from below the ears and muscle artefacts on the face.

FIG. 5 shows a possible user interface for client-side software used in a client-server model for providing users with, access to EEG or MEG data cleaning software that utilizes the disclosed technology for estimating the anatomical location of detected artefacts and providing supervised artefact rejection. In this figure, arrows indicate semi-sphere volume estimated components that are in the approximate locations where the eyes would be suggesting that these potential artefacts identified by the data cleaning algorithm relates to the eyes and not the brain. The insert of this figure is derived from real EEG data where the disclosed technology identified sources from the EEG pertaining to the eyes. Access to use this system may be provided through a subscription payment process in some embodiments.

### EXAMPLE 6

### Construction of Models of Brain Function that Shows the Coordination of Activities of Multiple Parts of the Brain that Takes MEG or EEG Data as Input and is Implemented as Either Stand-Alone Application Software or a Client-Internet Server or Cloud-Based System which is Used by University Brain Researchers, QEEG Practitioners, Medical Personnel, Game Developers, Training Simulation Developers, Investigators Using Neuromarketing, Neurocinematic or Neuroeconomic Methods, or Other Persons

In the recent years there are have been many new emerging companies using biometric data such as EEG and MEG data in the design of their products and services and the number of companies continues to grow. These companies products and services range from: (a) creating applications to understand how our brains and our behaviour are affected by various types of advertising and how people make decisions and how a person's decisions can be manipulated (persons using neuromarketing, neurocinematic, or neuroeconomic methods), (b) applications to control computers for the purpose of computer gaming and general human-computer interaction. Other companies such as computer game designers are interested in how well their game engages the player. Still other companies and organization are using EEG and MEG methods for investigating brain function with respect to stimuli (university brain researchers), while still others are using EEG and MEG methods for diagnostic purposes in their private practices and public health facilities (QEEG practitioners and hospital personnel).

In all of these cases, the disclosed technology can be used to identify in detail the systems of the brain and provide users of the disclosed technology with information about how their application relates to specific function of the brain. For example, in the case of neuromarketing, it is an investigative question of what systems of the brain are affected by the various visual and auditory events in a movie or commercial. This is because the neuromarketing goal is to determine if the current version of their movie or commercial elicits a positive affective response in the viewer with the end goal of selling more movies or selling more products using commercial advertising. If the brain function response is not suitable, they will adjust aspects of the movie or commercial until the desired brain function response is elicited. The case is analogous for neuroeconomic purposes. The disclosed technology is helpful in investigations of computer gaming so that developers can identify how to optimize their games such that they can elicit activities of specific areas of the brain and specific cognitive function. This is useful because one goal of developing games is to create a positive user experience, or to train a user how to use a particular part of their brain to solve a problem, or to use the game in a health-exercise or health-diagnostic application. QEEG practitioners and hospital personnel in contrast are interested in investigating brain function to identify brain damage, disease, or dysfunction. A QEEG practitioner uses EEG methods to detect the presence of problems such as ADHD, and then makes recommendations of how to address the problem. Similarly, the disclosed technology can help hospital personnel with brain function diagnostics to determine what systems of the brain are functioning or have been rendered non-functional by an accident.

The disclosed technology can be made available for all of these different applications via either stand-alone application software or a client-internet server or cloud-based system. As an example, the use of the disclosed technology can support an online internet data processing portal that mines EEG or MEG data. It then can provide a data-driven model of brain function with respect to the behavioural task during which the data were collected is described below as a data mining service.

Example steps for using the disclosed technology as part of a client-server EEG or MEG data mining service are given below. Similar steps would be used in a cloud-based implementation and a subset of similar steps would be used in a stand-alone application.
1. the user executes the client application and the client application connects to the server
2. the user of the system sends EEG or MEG data to the server to be cleaned
3. the user of the system also sends data describing events of the behavioural task (such as trial start and end events) and events of participant behaviour (such as button presses, joystick movements, galvanic skin response, heart-rate, or eye-gaze or saccadic movements) to the server
4. the user specifies any required input parameters through the client to the server such as data sampling rate, or others
5. the user specifies which auxiliary algorithms should be used to process the numeric output of the disclosed technology such as event-related desynchronization, or whether or not sub-groups from the main group should be identified, or if outliers should be identified and removed, or group consistency
6. the user begins the data processing function of the server via the client application
7. the disclosed technology processes the EEG or MEG data that were uploaded to the server and generates numeric and graphical results
8. the server provides numeric results with respect to the behavioural task events and with respect to the participant behaviour events for download or to the client application
9. the server provides graphical results with respect to the behavioural task events and with respect to participant behavioural events for download or to the client application
10. These results can include but are not limited to: (1) depiction of the location of active brain volumes, the activities of these volumes and the coordination of their activities on a 3D head model with respect to events specified by the user of the software or events automatically detected (this could be a difference between experimental conditions or for a single condition), (2) depiction of activation waveforms corresponding to components that could be instantaneous power or voltage or RMS power, or estimates of coordination such as zero-lag correlation, (3) depiction of scatter plots of numeric data depicting the relative distribution of brain function for multiple participants, (4) depiction of consistency of numerical output across the a group of participants, (5) depiction of classification results identifying subgroups within a larger group, (6) schematic diagrams of brain function derived from the numeric data showing relationships between processing functions with respect to task events, (7) depiction of the curves of convergence of volume-domain characteristics of components, (8) depiction of the ranked components and the automatically determined threshold separating those components with good volume characteristics and those that do not. Examples of some of these depictions of results are given in FIGS. 37, 38, 39, 40, 41, 42, 45, 46, 47, 48, 49, 51, 52, and 54.

FIG. 6 shows an example user interface for client-side software used in a client-server model for providing users such as university brain researchers, QEEG practitioners, medical personnel, game developers, computer-based training simulation developers with automated brain function model construction to reveal activities of systems of the brain in relation to their application. Access to use this system may be provided through a subscription payment process in some embodiments.

### EXAMPLE 7

### Analysis of Brain Function in Conjunction with Neurofeedback for the Purpose of Showing what Systems of the Brain are Affected in the Neurofeedback Session and the Lasting Effects of Neurofeedback Outside of the Session

The EEG biofeedback industry has created a variety of ‘protocols’ or combinations of visual, auditory, or tactile stimuli for the purpose of feeding back to the brain, via sensory input, information about the brain's function. This principle is that if the ‘brain’ and the biofeedback user can become aware of characteristics of how the biofeedback user's brain is functioning, then the user can gain the ability to bring their brain into desired operating states without requiring the biofeedback device. Such ‘brain states’ can be used to decrease the symptoms of such dysfunctions as attention deficit hyper activity disorder, or to improve language learning and comprehension, or other bring the user awareness of how to use their brain for other types of information processing. One problem with biofeedback methods is that it is not easy to obtain empirical evidence of what areas or systems of the brain a particular biofeedback protocol is targeting. Having a method to determine what areas or systems of the brain a protocol is affecting will help guide the improvement of existing protocols and the development of new protocols. It can also provide the ability to determine if a particular biofeedback protocol is actually effect.

The disclosed technology can be used to examine the brain function associated with at least three aspects of neurofeedback. First, it can be used during the neurofeedback training session to show the direct impact of the neurofeedback protocol. Second, it can be used while measuring brain function related to other task behavior outside of the neurofeedback session for the purpose of determining if the biofeedback training is affecting brain function outside of the training sessions themselves. Third, feedback may be generated based upon the measured activity of components corresponding to specific regions within the brain.

The steps for measuring brain function for the cases described above are similar. The main difference is that in the case of measuring the direct effects of neurofeedback, the task that the participant is engaged in is the neurofeedback task. In the case of measuring the effects of neurofeedback outside of the feedback session, the task may be an alternative task, for example a simple task such as eyes-open/eyes-closed or a more complex task such as interacting with a video game environment for the purpose of testing function of specific systems of the brain. Data could be collected for a group of participants to show how a particular neurofeedback training protocol affects brain function in a population, or data could be collected for an individual participant to assess their individual response and brain function changes.

The following are example steps that may be used to collect data.
1. Position the participant and connect equipment according the description in the Standard Equipment Configuration section of this document
2. Begin recording EEG or MEG data
3. Begin the either a paradigm to analyze the lasting effects of neurofeedback using a set of tasks to assess brain function or a neurofeedback paradigm.
   a. The tasks to assess the lasing effects of neurofeedback on brain function could be:
      i. Simple Task: eyes-open/eyes-closed
      ii. Complex Task: activate systems of the brain that the drug is designed to affect, and
      iii. Complex Task: activate systems of the brain that the drug is not designed to affect
4. Upon completion of the paradigm, stop all recording and store the data for later data analysis
5. Repeat steps 1 to 4 until all participants have contributed data

The following are example steps that could be used to analyze data.

To analyze the collected data using the disclosed technology, the example steps below may be followed.
1. Use the disclosed technology to process the recorded MEG or EEG data for all participants with respect to the recorded events in the task software relating to the neurofeedback protocol or a different protocol for assessing brain function outside the neurofeedback session and data containing behavioural participant responses. See FIG. 39 for example brain source identification for 1 participant. See FIG. 51 for example brain source identification for a group.
2. To evaluate how brain function is affected in the neurofeedback session when group data are available, use the numeric output (for intervals of data related to key moments in the behavioural task) calculate
   a. activation of brain areas
      i. create scatter plots and from these scatter plots visually identify outlier participants or subgroups within the participant data. See FIGS. 45 and 46. (Alternatively, use an algorithm such as the kmeans algorithm for identifying outliers and subgroups within the participant data.)
      ii. create plots of consistency of activation across the group. See FIG. 47.
   b. consistency across the group
   c. coordination among brain areas
      i. Create scatter plots and from these plots visually identify outlier participants or subgroups within the participant data (or use automated methods of grouping and outlier detection)
3. To evaluate how brain function is affected in a neurofeedback session when only 1 participant dataset is available (for intervals of data related to key moments in the behavioural task) calculate
   a. activation of brain areas (See FIG. 40.)
   b. coordination of brain areas (See FIG. 41.)

### Test Results

This section describes example applications of the technology that have been used in research to successfully generate results.

### Case Illustrating Correct Operation of the Spectral Shaping ICA Data Mining Algorithm

This application demonstrated that statistics of scalp-EEG can differ across the EEG frequency spectrum and that shaping of the EEG frequency spectrum prior to ICA decomposition can improve estimates of source activities and separate EEG data into more anatomically specific components.

The Band-Selective ICA (BSICA) algorithm was used in conjunction with the runica ICA algorithm to calculate a frequency spectrum shaping filter that emphasizes frequencies of statistical independence. This filter was used to shape the frequency spectrum of EEG data prior to using runica, a second time, to calculate new spatial source separation filters. These spatial source separation filters were used to decompose unshaped EEG data into its component parts. Components calculated by these steps were compared to components obtained by standard application of runica. Differences between the results were identified by examining characteristics of component topography, modelled physical brain volume overlap, and pair-wise time-domain correlation of activities as a function of frequency.

The spectral shaping method separated bilateral activities of the EEG into two distinct, anatomically correct components (that standard ICA methods did not) and visibly improved the topographical representation of two other contributors to the EEG. Spectral shaping brought about statistically significant changes to the pair-wise correlation of brain activity components (as compared to the standard runica method) as a function of frequency for the frequency bands 2-18 Hz (increased correlation) and 20-38 Hz (decreased correlation), assigning the greatest correlation to the low frequencies of the brain activity frequency spectrum. Shaping also significantly decreased the pair-wise correlation of brain activity component activities in the 40-76 Hz frequency band to appropriately assign the lowest level of correlated activity of the EEG spectrum to the frequency band thought to contain non-brain activity related noise. Shaping provided no significant changes to the physically modelled overlap of estimated brain volumes.

The statistics of the scalp EEG differ according to the frequency band examined. Further, the proposed spectral shaping method preserved the real correlative structure of brain source activities and separated the contributors to the EEG into more anatomically specific parts better than the standard method.

Decompositions of EEG data by each method yielded 30 components with corresponding waveforms and topographies. Subsequent projection of topographical variance to the volume domain via the volume-projection algorithm yielded a volumetric spectrum corresponding to each component.

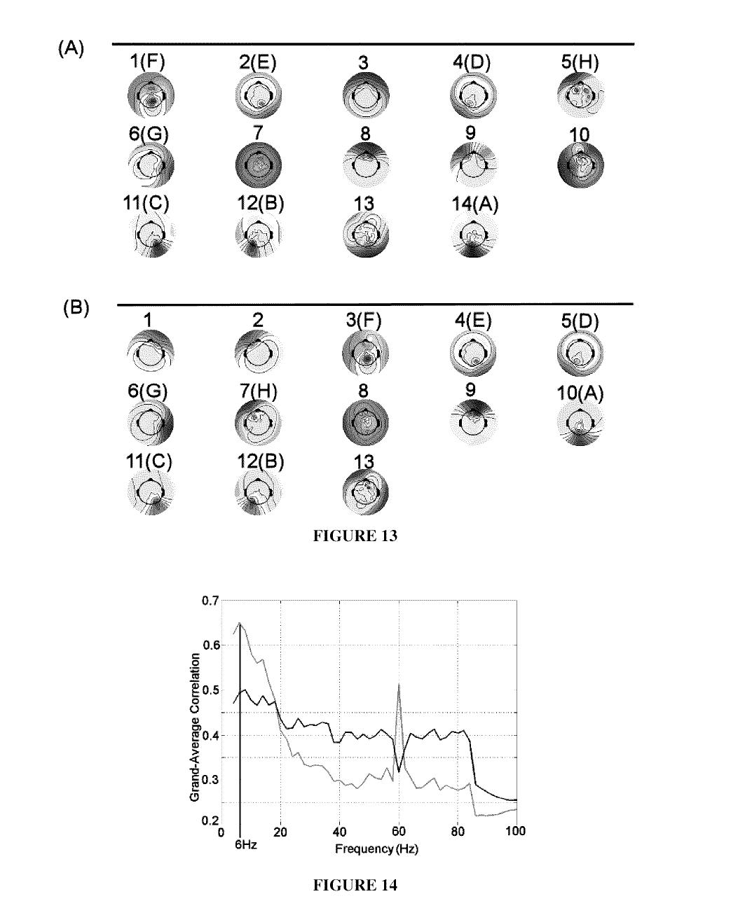

Components have been numbered 1 through 30 according to their descending PSV scores, separately, for each decomposition method. When sorted by their PSV scores, most noise-related components were separated from possible brain-related activities. The topographies of components above the threshold used to separate artefacts from possible brain activity sources are plotted in FIGS. 13A and 13B. EEG components topographies calculated using the standard method are given in FIG. 13A while those calculated using the shaped method are given in FIG. 13B.

FIGS. 13A and 13B show topographies corresponding to EEG components calculated by standard ICA (FIG. 13A) and ICA involving spectral shaping (FIG. 13B). Topographies are sorted in descending order according to PSV rank. Only those components greater than the cut-off threshold are shown. Components are cross-labelled by letter to retain their PSV sorted rank and component matching for standard and shaped components/topographies. Dark regions indicate maximum intensity. Light regions indicate neutral or zero. Component topographies have a sign ambiguity when they are evaluated independently of their waveform activity. In the current figure, relative polarities as positive or negative are not important when making comparisons between topographies.

By comparing components according to their topographies across decomposition methods, a set of 8 common dipolar components was identified and cross-labelled as (A) to (H) (FIG. 13) to be used to compare decomposition characteristics of the two methods. Component 8 of the standard method and component 9 of the shaped method, although they match across decomposition methods and they fit our criteria of a dipolar component, were not cross-labelled and included in the methods comparison.

Visible differences (FIGS. 13A and 13B) between the topographies Aⱼ of the cross-labelled components suggest improvement was provided by the spectral shaping method over the standard method. Topographies F and H, calculated using the spectral shaping method, are visibly smoother and more dipolar (using the definition given in the introduction of this paper) for the spectral shaping method than those calculated via the standard method. This is most clearly visible for component H where better separation of a left hemisphere contributor from possibly 2 right hemisphere contributors to the EEG is evident.

Three noise components from the standard method and two components omitted from the spectral shaping method that were not eliminated via the PSV threshold were eliminated because they did not meet the dipolar criteria. These were components 7 from the standard decomposition method and 8 from the spectra shaping decomposition method and components 10 and 13 from the standard decomposition method with topographies that are similar to component 13 from the spectral shaping decomposition method.

Two unique components calculated by each decomposition method did not have matching topographies between decompositions but did have dipolar topographies and warranted further investigation. Similarities of location of topographical foci (neglecting polarity of the foci) for the topographies of components 3 and 9 of the standard decomposition and 1 and 2 calculated using the spectral shaping decomposition method suggest that these components from the two decompositions are related. Components 3 and 9 of the standard decomposition could thus be describing common origins of activities. Component 3 of the standard decomposition is characteristic of an in-phase, anterior bilateral source pair and appears to be related to the bilateral pair of components 1 and 2 of the spectral shaping decomposition. Component 3 might also be and in-phase version of the out-of-phase component 9 of the standard decomposition. This speculation is supported by the absence of components similar to component 3 and 9 from the spectral shaping decomposition and the presence of components 1 and 2.

The grand-average pair-wise correlation spectrum of components obtained using the spectral shaping method has a greater range of correlation than that of the standard method. This difference is illustrated in FIG. 14 which shows average pair-wise correlation spectra for the standard (black) and shaped (grey) decomposition methods.

This range of correlation of components activities calculated using the spectral shaping method has a shape more similar to the power spectrum of scalp EEG than that of the standard method, with maximum correlation at low frequencies (<20 Hz) and minimum correlation at high frequencies. There is a peak correlation of 0.65 at 6 Hz, with a gradual decrease in correlation to approximately 0.3 at 36 Hz. The correlation spectrum is relatively flat at approximately 0.3 in the interval 36-85 Hz (excluding a peak at 60 Hz). In contrast, the average pair-wise correlation spectrum for the standard decomposition is relatively flat with correlation equal to 0.4 between 20 and 85 Hz. However, it has a peak correlation of 0.5 between 2 and 20 Hz. For the spectral shaping method and the standard method, the correlation is low (consistently <0.3) for frequencies greater than 85 Hz.

The grand-average pair-wise correlation spectra of the shaped method indicate that there are distinct regions where correlation of brain activities exists and where correlation does not. It demonstrates that areas of the brain have varied levels of correlated activities in the frequency band 2-38 Hz, whereas at frequencies higher than 38 Hz, there is little to no correlation relating to brain activity. Frequencies greater than 38 Hz illustrate the noise floor indicating uncorrelated activity. There is a clear average difference between correlation activities of the band 2-20 Hz and 20-40 Hz.

A 60 Hz peak that is likely AC power noise in the grand-average pair-wise correlation spectra illustrates that the spectral shaping method does not suppress the correlated 60 Hz noise activity present in each component. Conversely, for the standard decomposition there is a notch in the correlation structure at 60 Hz. This AC power noise is ever present in the environment and its visibility in this result is not unreasonable.

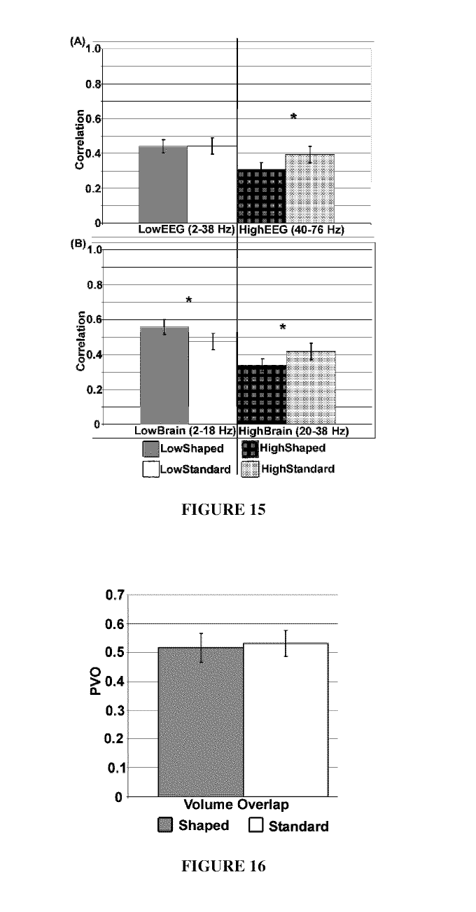

The plots of FIG. 15 show the average pair-wise correlation level calculated across components of each decomposition method. FIG. 15 shows summarized pair-wise correlation of components for various frequency bands comparing the shaped and standard methods. Error bars indicate standard error. Statistically significant differences are indicated as (*). (A) is a comparison of shaped and standard methods for the EEG frequency spectrum divided into two bands, LowEEG (2-38 Hz) and HighEEG (40-76 Hz). The difference between the methods for the HighEEG band is significant (p=0.0201) for p<0.05. (B) is a comparison of the shaped and standard methods for the brain activity portion of the EEG spectrum. There is a significant difference (p=0.0232) for LowBrain frequency band (2-18 Hz) and a significant difference (p=0.0159) for HighBrain frequency band (20-38 Hz) for p<0.05.

Significant differences between decomposition methods for the pair-wise correlation spectra of the high EEG frequency band are indicated by the plots of FIG. 15A that compare the brain (2-38 Hz) and non-brain (40-76 Hz) frequency intervals. The average correlation level of the shaped spectrum is significantly lower than that of the standard spectrum (p=0.0201) for the high frequency band (40-76 Hz). For the low frequency EEG band (2-38 Hz), there is no significant difference (p=0.933) between the average correlation level for the two decomposition methods. This indicates that the activities in the high frequency EEG band are uncorrelated.

The plots of FIG. 15B, illustrate differences between the decomposition methods. These plots show that on average, the standard decomposition method obscures the correlative relationships between activities of different parts of the brain and that the shaped method preserves them. The shaped method provides a statistically significant increase (p=0.0232) in the pair-wise correlation of the low frequency brain activity band (denoted in the figure as ‘LowBrain’) (2-18 Hz) and a significant decrease (p=0.0159) in the high frequency brain activity band (denoted in the figure as ‘HighBrain’) (20-38 Hz) compared to the standard method. This result indicates that the low frequency brain activity band has higher correlation for the shaped method than the standard method while for the high frequency brain activity frequency band the shaped method has lower correlation than the standard method.

FIG. 16 shows that the spectral shaping method has a lower average PVO than the standard method. FIG. 16 is a comparison of the pair-wise volume overlap (PVO), ranging from 0 to 1 for components common to both the shaped and standard decomposition methods showing that there is on average no statistically significant difference. A value of 0 indicates no overlap while a value of 1 indicates maximum possible overlap. Error bars are the standard error. A t-test examining the difference between methods yielded a non-significant difference (p=0.56) in the pair-wise volume overlap properties indicating that this decrease was not significant and that there is no substantive difference in the volume representations of components.

The plot of FIG. 17A shows that components 1 and 2 of the shaped decomposition method pertain to a pair of bilateral modular volumes. These comprise a bilateral source pair located in the orbital regions of the skull beneath the left and right poles of the frontal lobe of the cerebral cortex. Visual comparison of calculated modular volumes of these components with fMRI results examining activities of the orbital muscles of a different study provides corroboration that these two components represent the activities of the orbital muscles of the right and left eyes, respectively. This result shows that the spectral shaping method correctly identified these sources contributing to the EEG as two distinct entities. The standard decomposition method does not yield similar component topographies that can be localized as similar source volumes and thus it does not appropriately separate the ocular muscles activities contributing to the EEG. The ocular movement activities contributing to the EEG were, in fact, separated into an ‘in-phase’ component and an opposite-phase component as visible by their topographies (see components 3 and 9 of the standard decomposition in FIG. 13A).

The pair-wise cross correlation of components 1 and 2 of the shaped decomposition illustrated in FIG. 17B shows a distinct notch in the pair-wise correlation at 34 Hz. There is also a peak in the correlative structure at 60 Hz and a general increase in pair-wise correlation from 2-8 Hz. Excluding the 34 Hz frequency band, the correlation is approximately flat between 8 and 84 Hz with an average correlation of approximately 0.46. This result shows that the spectral shaping method can successfully separate the activities of two components that have a relatively high correlation at most frequencies except for a single narrow frequency band.

### Example Illustraing Correct Operation of the Volume-Domain Projection and Volume Estimation Algorithm Using Synthetic EEG Data

We present a methodology that relates topographies calculated via Independent Component Analysis (ICA) of EEG data to volume domain representation not constrained to a single voxel. We investigate the feasibility of using such projections to estimate brain source volumes and volume overlap of source pairs, and determine if neural and artefact sources might be differentiated by their volume domain characteristics.

Synthetic EEG data were comprised of artefacts and multiple sources within a head model and decomposed into parts using ICA. An adaptation of Linearly-Constrained Minimum-Variance Beamforming, projected ICA-derived topographies into a model brain volume. Additive noise was used to meet the Beamformer requirement for an EEG baseline to remove volume domain bias. Estimates of volume domain noise were used to calculate the edges of modular brain volumes.

FIG. 18 shows two views of the head model with simulated sources. Arrows at each source voxel indicate positive source dipole direction. Three functionally distinct, single-voxel sources are proximally spaced in the right anterior temporal pole. A single 26-voxel source is modelled in the left orbital frontal pole. A single-voxel source located in the right parietal region models a distally spaced source. The scalp is shown with superimposed projected field polarities originating from the model sources. From the location and orientation of the placed sources in this depiction and the time-domain activities of each model source the synthetic EEG mixture was created.

Volume projections calculated from synthetic EEG data are similar to actual model source volumes. Estimates of volume modularity are similar to actual volumes for a threshold of 5 standard deviations above the mean volume noise estimate. Components relating to model brain sources placed at unique proximal and distal locations have non-overlapping volumes and high volumetric spectrum peaks. Modelled artefact components have low volumetric spectrum peaks and overlapping volumes.

The disclosed methodology provides a basis for linking time and topography domains of ICA components to physical volume domain representations. Further, volume domain projections provide source information that might be used to differentiate brain activity from artefact components.

A mathematical head model was constructed to both create synthetic EEG data and to evaluate the effectiveness of the proposed method. Matlab 7 (Mathworks Inc.) and EEGLab 4.515 were used to do all modelling, calculations, and plotting. BERG head model parameters and artwork for the model were derived from the BrainStorm software package for Matlab. The BERG parameters used in the three-layer head model are listed in Table 1. The head model was calibrated with a volumetric grid/voxel resolution of 0.5 cm³ and implemented using the equations provided in the previous sections. 5439 voxels were used to define the volume of the head model in this study. Localization was not constrained from occurring in the ventricles or other physiologically improbable areas, following the philosophy of minimal assumptions in the localization process.

TABLE I
Parameters used for construction of the head model calculated
using the BrainStorm software package.

| | Center | Radii | sigma | Mu | lamda |
|---|---|---|---|---|---|
| Layer1 | 0.0083 | 0.0899 | 0.3300 | 0.5732 | 0.5195 |
| Layer2 | 0.0006 | 0.0949 | 0.0042 | 0.0394 | 0.0275 |
| Layer3 | 0.0489 | 0.1025 | 0.3300 | 0.9464 | 0.1441 |

‘Center’ is the center of the head.
‘Radii’ is the radii of the sphere.
‘sigma’ is conductivity.
‘Mu’ defines the BERG eccentricity factors.
‘Lamda’ defines the BERG magnitude factors.

We defined three physical source characteristic scenarios by strategically assigning source properties. The first scenario's single-voxel source is placed in the central region of the right hemisphere, distal from all other sources, providing the simplest case for source separation and volume estimation. This is labelled as source (1). This thus created the case where topographical comparisons of source may not provide a good measure of the difference between individual sources. The second scenario examined difficult source separation with a high likelihood of volume overlap when source separation is imperfect. This entailed a group of single-voxel sources, proximally placed in the right temporal pole. These are labelled as sources (2) through (4). Each source was assigned the same orientation, differing from that of source (1). The third scenario examined the volume estimation for a trapezoidal multi-voxel source. This 26-voxel source was placed in the left orbito-frontal region, distally from other sources. All voxels comprising this source were assigned the same orientation, differing from all other sources. FIG. 18 illustrates the source locations and orientations on a head model used

The topographical projections of each model source were calculated to create simulated scalp-EEG data and to later compare ICA-derived topographies of each source to their original counterparts. These were calculated by projecting each modelled volume source onto a model scalp, as described in the forward solution of Equation 6, for a given dipole orientation.

The time-domain activities of simulated EEG data were created by combining the dipole projected topographies with synthetic waveforms and additive uncorrelated sensor noise. Distinct waveforms were assigned to each source for easy visual identification in plots. Sources (1) through (5) were assigned the waveform activities of a ramp, a sinusoid, a random uniformly distributed waveform, a square wave, and a waveform with a random super-Gaussian distribution, respectively. All neural sources were set to have zero mean and unit standard deviation in each trial. Each source waveform was projected to the scalp-domain using the topographies calculated via model dipoles in the previous step. The topographically projected activities were summed to create 124 channels, 875 samples/trial, and 29 trials of simulated EEG data with source activities repeating in every trial. Random, uncorrelated sensor noise was added to each trial of simulated EEG after mixing simulated neural sources. Sensor noise amplitude was set to 28 dB below the average source volume levels.

Electrode artefacts were added to the simulated EEG to investigate possible characteristic volumetric spectra that might differ from simulated neural sources. Electrode artefacts having an amplitude approximately 60 dB greater than the simulated neural sources were modelled at one electrode at a location near the apex of the scalp. The artefact was a spike event occurring in every trial at a random latency. A block diagram illustrating the construction of the simulation data is given in FIG. 19.

Multiple waveforms and topographies were calculated from the EEG mixture using ICA. The extended runica algorithm available in the EEGLab 4.515 analysis package was used to separate the simulated EEG into parts. An initial PCA step to reduce the rank of the data from 124 to 8 was used to facilitate convergence of the runica algorithm. This intentional over-estimate of the number of sources comprising the data simulated real analysis situations when the actual number of sources is not known. (It has been recommended to maximally separate the data using all available degrees of freedom thus it is appropriate to simulate an over-estimate of rank.) Both the original model source waveforms and those waveforms recovered using the proposed method were mean-subtracted and normalized to unit standard deviation. As well, the original and recovered topographies were mean subtracted and normalized to have unit norm. The waveforms and topographies were plotted for visual comparison.

In order to calculate differences between the actual model and estimated centers of mass for each of the single-voxel sources (1) through (4), from the synthetic EEG, the difference between the x, y, z coordinates as in Equation 28 was calculated. The actual model centers of mass and estimated centers of mass are x₀, y₀, z₀ and x₁, y₁, z₁, respectively.

error=\sqrt{(x₀−x₁)²+(y₀−y₁)²+(z₀−z₁)²} (28)

The error associated with the center of mass of source 5 (the volume source comprised of 26 voxels) was not calculated because its center of mass is not on the head model grid. As designed, the volume source was originally comprised of 27 voxels arranged as 3×3×3, placed on the edge of the white-matter frame. Modular volumes represent active grey matter while the white matter framework provides a physical frame of reference so that the resolved volumes can be named anatomically. One of the voxels however was truncated from the model volume during processing for being too close to the edge of the head model, thus leaving only 26 voxels for evaluation. Fortunately, this does provide the model with the case when the center of mass of a source is not precisely on the grid.

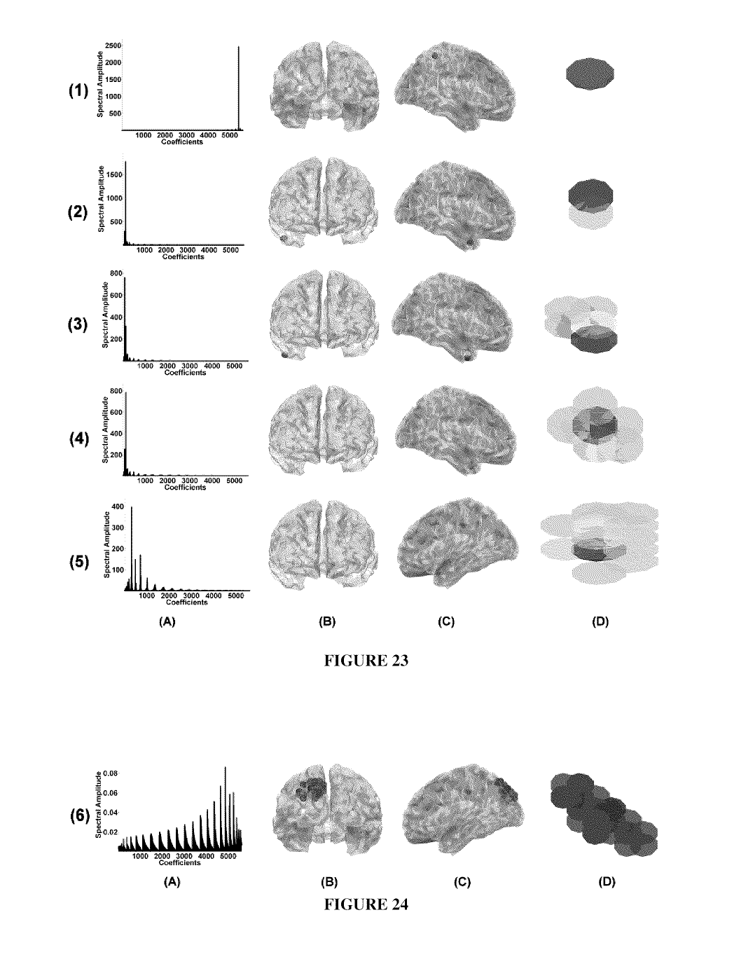

Source center of mass estimates were plotted on the head models of FIG. 23. They are indicated as the peaks of the volumetric spectra. Associated errors were tabulated.

Modular Source volumes were estimated using the thresholding method described in the previous section. We assumed a threshold of 5 standard deviations above the mean (STDM) as an appropriate confidence interval for making source volume plots base on previous experimentation with different sources during the development of the volume estimation algorithm. To relate the thresholded source volume plots to a continuous measure of volume estimate, the modular volumes of each source with respect to multiple threshold levels from 3 to 6 STDM were calculated (FIG. 26). FIG. 26 shows estimated source volumes for 3 STDM to 6 STDM. The recovered EEG components (1) through (8) are indicated on the plot. Components (1)-(5) are simulated neural sources, (6) is an electrode artefact, and (7) and (8) are rank-error components. The source volume estimate error was calculated as the difference between the number of voxels comprising the model versus the number of voxels comprising the estimated source as in Equation 29,

error=|v₁−v₀| (29)

where volumes v₀ and v₁ are the actual volume and the estimated volume, respectively. The original and recovered source volumes with associated error are listed in Table 2.

Overlap was calculated according to Equations 30 and 31.

Each raw volumetric spectrum was mean-subtracted and normalized for unit norm in Euclidian space according to Equation 30,

$$\hat{r} = \frac{r - \overline{r}}{\sqrt{{( {r}_{i} - \overline{r} )}^{2} + {( {r}_{2} - \overline{r} )}^{2} + … + {( {r}_{p} - \overline{r} )}^{2}}} \qquad ( 30 )$$

where r̅ is the mean of all rᵢ elements of r. Each r has 1×P elements. The resulting zero-mean normalized volumetric spectrum is designated as \hat{r}.

The overlap was calculated between pairs of the zero-mean, normalized volumetric spectra pertaining to the recovered topographies as in Equation 31,

S=|\hat{r}ᵢ\hat{r}ⱼ^{T}| (31)

where rᵢ and rⱼ are the zero mean, normalized volumetric spectra compared. This volume overlap, S is a measure of the similarity between the volumetric spectra.

For example, zero overlap of two arbitrary vectors u and v is evidenced by a value of 0. A value of 1 indicates maximum overlap of u and v. The results were tabulated for later inspection.

To check the correctness of the volume projection component of the algorithm, the pair-wise overlap of the raw volumetric spectra for each of the original model topographies used to create the simulated EEG were tabulated and compared. This was done in a similar manner to that of the recovered volumetric spectra, using Equations 30 and 31. This also created an instance of idealized volumetric spectra (with errors only relating to mean subtraction of the topographies) by which the recovered volumetric spectra of the ICA-derived topographies were compared, also using Equations 30 and 31. Similarly, a comparison was made between the original model topographies and the recovered topographies.

Spectral peak characteristics of all ICA-derived components were tabulated and compared to identify a possible volume-domain characteristic that can be used to distinguish between EEG components relating to neural sources, components relating to electrode artefact, and components resulting from ICA rank estimate error.

The resulting decomposition contains 5 components pertaining to simulated neural sources, 1 component relating to electrode artefact, and 2 components resulting from the intentional rank estimation error. Sources resulting from the rank estimate error are herein referred to as ‘rank error’ components.

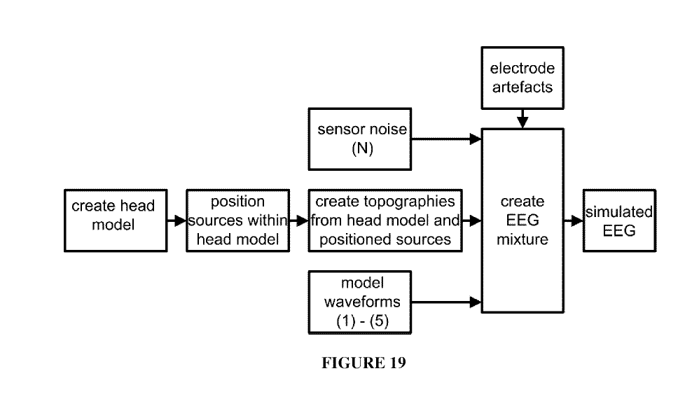

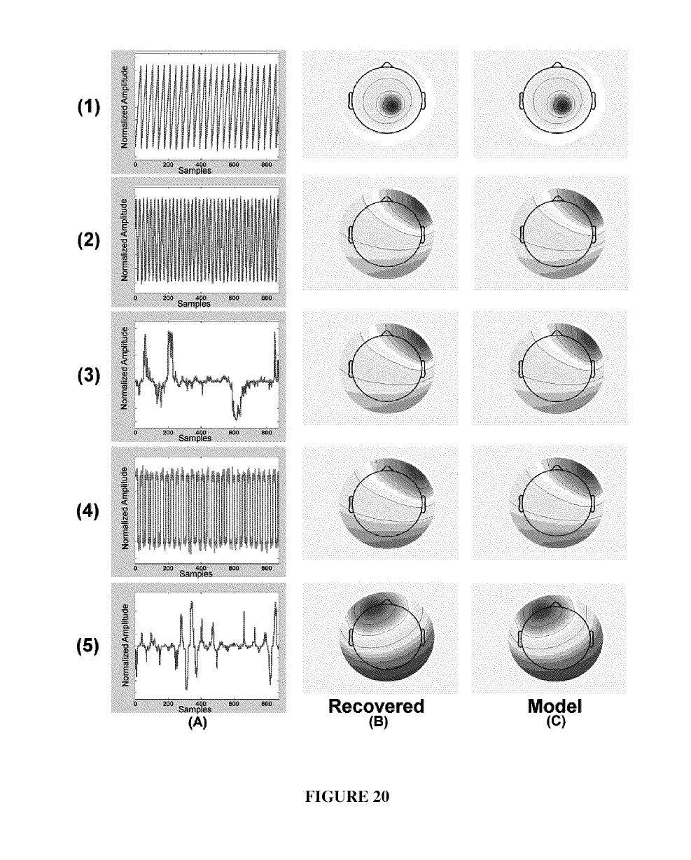

The topographies and waveforms of EEG components (1) through (5) recovered using ICA are plotted in FIG. 20 with the model topographies and waveforms used to construct the stimulation data.

FIG. 20 shows original and recovered topographies and sources (1) to (5). Original model waveforms (dark dotted) and recovered waveforms (light solid) associated with each topography are given in column (A). In cases where only a single waveform appears to exist, the original and recovered waveforms are overlapped. Recovered topographies are given in column (B) while original model topographies are given in column (C).

Plots show the trial-averaged waveform. Visual comparison of the recovered sources (1) to (5) reveals that unmixing and trial averaging produces little distortion. Proximal temporal source waveforms for (2), (3), and (4) demonstrate separation of activities with most visible distortion for (4). The distal source waveform for (1) and the multi-voxel source waveform for (5) also have little distortion. There is no apparent distortion when comparing topographies.

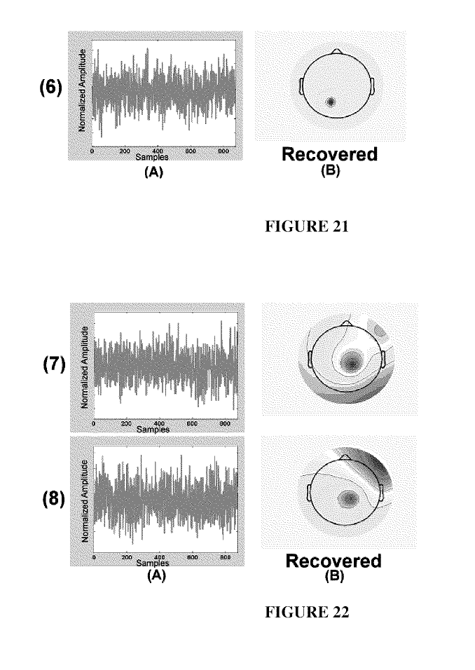

The electrode artefact (6), and associated waveform extracted from the EEG, is given in FIG. 21. FIG. 21 shows extracted electrode artefact component (6) added to the EEG. The recovered waveform is given in column (A). The recovered topography is given in column (B).

The plots of FIG. 21 show the trial-averaged waveform. The position of the focal point in the topography of (6) corresponds to the single electrode at which artefacts were simulated. The corresponding waveform reflects the average spike activity temporally distributed over the entire epoch of the averaged data.

Component topographies and waveforms related to rank estimate error (7) and (8) are given in FIG. 22. FIG. 22 shows extracted ICA rank error components (7) and (8). Recovered topographies are given in column (B). Recovered waveforms are given in column (A). Artefacts (7) and (8), resulting from the attempt to separate the simulated EEG into more parts than actually comprise the data, have topographies that resemble combinations of simulated sources. The waveforms corresponding to these components do not clearly represent any one source. Plots show the trial-averaged waveform.

There was no volume estimate error for the distally spaced source. For proximally spaced single-voxel sources and for the multi-voxel source, only small volume estimate errors were present. There were no center of mass errors for the sources examined. The results of the source location and volume estimate for a 5 STDM threshold are summarized in Table 3 and illustrated in FIGS. 23, 24, and 25. The volumetric spectrum is given in column (A) of each figure. The locations and volumes with respect to the model head are given in columns (B) and (C) of each figure. Source centers are indicated by the peak of the volumetric spectrum, as shown by the single red sphere in the head model plots. Where applicable, column (D) gives a magnified depiction of the estimated volumes.

FIG. 23 shows localization and volume estimate results for simulated neural sources (1) to (5). (A) volumetric spectra. On the vertical axis, spectral amplitude indicates the values of the coefficients of the volumetric spectrum. Each coefficient defining the three-dimensional volume is plotted as a vector of values on the horizontal axis; (B,C) estimated volumes in relation to the head model; (D) magnified side-view of volume estimates. Fall-off from the spectral peak is reflected in the color of the spheres, ranging from dark-color (corresponding to the peak of the volumetric spectrum) to light-color (corresponding to the minimum of the volumetric spectrum).

FIG. 24 shows localization and volume estimate results for the electrode artefact, component (6). (A) volumetric spectra. On the vertical axis, spectral amplitude indicates the values of the coefficients of the volumetric spectrum. Each coefficient defining the three-dimensional volume is plotted as a vector of values on the horizontal axis; (B,C) estimated volumes in relation to the head model; (D) magnified volume estimates. Fall-off of coefficient values from the PSV is reflected in the color of the spheres, ranging from dark-color (corresponding to the peak of the volumetric spectrum) to light-color (corresponding to the minimum of the volumetric spectrum). In this case, only dark-color spheres are present as the spectrum does not have a sharp fall-off, indicating that there is very little difference between signal as a resolvable modular volume and the noise in the volumetric spectrum.

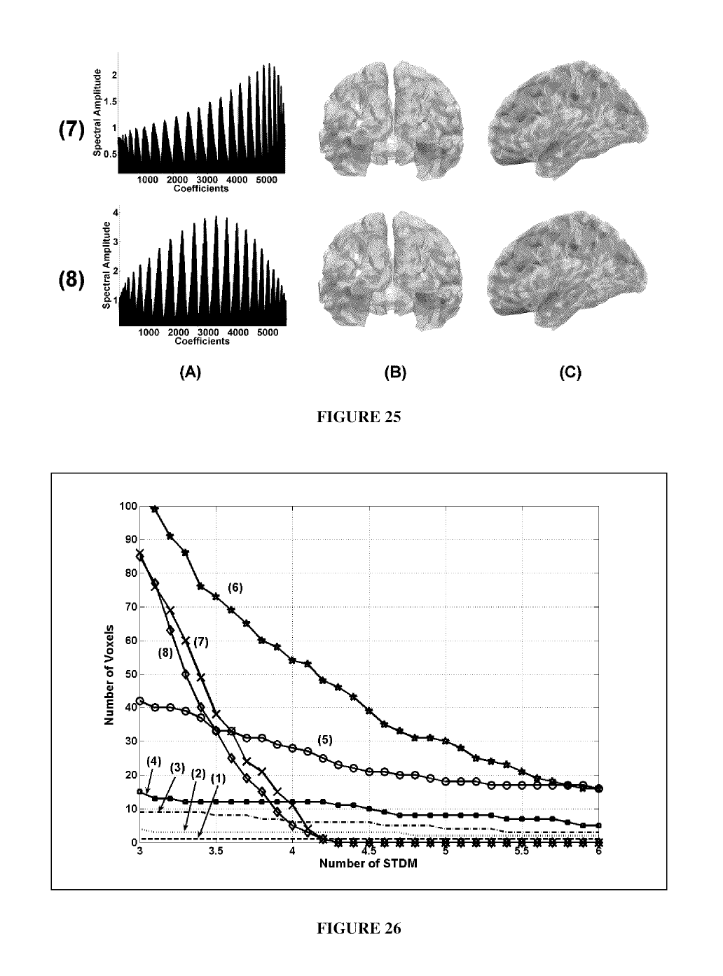

FIG. 25 shows localization and volume estimate results for rank-error components (7) and (8). (A) volumetric spectra. On the vertical axis, spectral amplitude indicates the values of the coefficients of the volumetric spectrum. Each coefficient defining the three-dimensional volume is plotted as a vector of values on the horizontal axis; (B,C) estimated volumes in relation to the head model. Absence of spheres indicates all spectrum coefficients were below the 5 STDM threshold.

A volume was estimated for the electrode artefact, component (6). This volume yields a peculiar spectral fall-off compared to the simulated neural sources making it distinct from the non-artefact sources. It has a characteristically blunt spectral peak and gradual fall-off from the peak. The volumetric spectra of the rank error components (7) and (8) are also blunt like the electrode artefact (indicated by the spectrum of FIG. 25a), however all coefficients were less than the threshold of 5 STDM.

TABLE 2
Source location and volume estimates at a 5 STDM threshold for each
type of simulated neural source. Units are given as number of voxels.
(The double dash indicates that a comparison was not made.)

| | Source | Recovered | | |
|---|---|---|---|---|
| | Volume | Estimate of | Volume | Location |
| | (voxels) | Volume (voxels) | Error | Error |
| (1) Distally spaced | 1 | 1 | 0 | 0 |
| (2) Proximally spaced | 1 | 2 | 1 | 0 |
| (3) Proximally spaced | 1 | 5 | 4 | 0 |
| (4) Proximally spaced | 1 | 8 | 7 | 0 |
| (5) Multi-voxel | 26 | 18 | 8 | -- |

The volume estimates at thresholds increasing from 3 to 6 STDM are plotted in FIG. 25. This volume-STDM plot shows that for different STDM thresholds there is an adjustment in the modular volumes estimated. There is a distinct difference between the curves associated with the rank error components (7) and (8), and the simulated neural components (1) through (5). The curve relating to the electrode artefact (6) has characteristics of both the neural components and the rank error components, but is different from neural source components in that the volume estimates are relatively stable over the 3 to 6 STDM threshold interval examined.

Overlap of the volumetric spectra pertaining to the spectra estimated from the ICA-derived topographies and the spectra pertaining to the actual model topographies are given in Table 3. With regards to the recovered spectra, there is a notable overlap between sources (2) and (3), and between the electrode artefact (6), and the rank error artefacts (7) and (8). However, there is approximately zero overlap between volumes for the original model spectra. An overlap of 0.540 was calculated for the pair-wise comparison of recovered sources (2)-(3). The pair-wise comparisons for recovered sources (2)-(4), and (3)-(4) gave an overlap of 0.0663 and 0.121, respectively. For the distally spaced sources (1)-(5), the maximum overlaps were 0.00722 and 0.00722, respectively (the overlap with each other). The overlaps of the volumetric spectra calculated from the model topographies were all less than 0.00346.

TABLE 3
Distance between volumetric spectra calculated for topographies. Distances
pertaining to volumetric spectra of recovered topographies are given in [ ],
while distances pertaining to the volumetric spectra of model topographies used
to create the simulated EEG are given in { }. Maximal difference is indicated
by 0, minimal distance is indicated by 1. Values greater than 0.3 have been highlighted
to emphasise values of low overlap from high overlap. Numbers across the top of the
table in parenthesis indicate component number. (The double dash indicates that the
corresponding comparison is in the upper half of the table. e.g, 2 vs. 3 = 3 vs. 2)

| | (2) | (3) | (4) | (5) | (6) | (7) | (8) |
|---|---|---|---|---|---|---|---|
| (1) | [0.00143] | [0.00242] | [0.00316] | [0.00722] | [0.0148] | [0.0757] | [0.00590] |
| | {0.000178} | {0.000178} | {0.000178} | {0.00346} | | | |
| (2) | -- | [0.540] | [0.0663] | [0.00460] | [0.0322] | [0.00911] | [0.0384] |
| | | {0.000141} | {0.000176} | {0.00297} | | | |
| (3) | -- | -- | [0.121] | [0.00559] | [0.0548] | [0.0107] | [0.0688] |
| | | | {0.000176} | {0.00307} | | | |
| (4) | -- | -- | -- | [0.00228] | [0.0705] | [0.00427] | [0.0994] |
| | | | | {0.00249} | | | |
| (5) | -- | -- | -- | -- | [0.114] | [0.0761] | [0.000614] |
| (6) | -- | -- | -- | -- | -- | [0.361] | [0.416] |
| (7) | -- | -- | -- | -- | -- | -- | [0.0127] |

Table 4 compares both actual and estimated topographies and volumetric spectra using the metrics defined by Equations 25 and 26. The greatest difference in volumetric spectra is with the multi-voxel source (5), followed by the proximally spaced single voxel sources. This is also true for the topographies. There is not however, a consistent trend between topographical error and error of the volumetric spectra. Thus the size of error in one domain can not imply the size of error in the other domain.

TABLE 4
The similarity between model vs recovered topographies and model
vs recovered raw volumetric spectra. Maximum similarity is indicated
by 1. Minimum similarity is indicated by 0. Mod. = Model;
Rec. = Recovered. Numbers in parenthesis across the top of
the table indicate component number. (The double dash indicates
a comparison that was not made.)

| | Mod. Vs | Mod. Vs | Mod. Vs | | Mod. vs |
|---|---|---|---|---|---|
| | Rec. | Rec. | Rec. | Mod. vs | Rec. |
| | (1) | (2) | (3) | Rec. (4) | (5) |
| Topography | 0.9999 | 0.9995 | 0.9994 | 0.9996 | -- |
| Volume | 0.9968 | 0.9793 | 0.8990 | 0.8797 | 0.8664 |

There is large variation in the maxima of the volumetric spectra across recovered components. The peaks are listed in Table 5 for each component of the EEG. The peaks of the recovered neural sources vary between 2,463 and 369.5. The peak for the artefact electrode is 0.08614 and for those components relating to the rank error, the peaks range from 2.212 to 3.868. There is at minimum two orders of magnitude difference between the recovered neural sources and the artefacts. The peaks of the volumetric spectra computed directly from model topographies range from 36,540,000 to 18,950,000. These peaks are extremely large and tend to increase towards infinity (as machine representation) because they indicate a near perfect voxel specificity of the topographies. In examining real data, such large numbers are unlikely to occur as an EEG-derived topography will contain at least some noise and will not necessarily have a volume that is defined and perfectly centered within a single 0.5 cm head model voxel.

TABLE 5
Peaks of the volumetric spectra for each of the model and ICA-derived topographies.
Peak Mod. = Peak of the model spectra; Peak Rec. = Peak of the recovered
spectra. Numbers in parenthesis across the top of the table indicate component number.

| | (1) | (2) | (3) | (4) | (5) | (6) | (7) | (8) |
|---|---|---|---|---|---|---|---|---|
| Peak Rec. | 2463 | 1777 | 761.0 | 786.0 | 369.5 | 0.08614 | 2.212 | 3.868 |
| Peak | 36540000 | 19390000 | 20030000 | 18950000 | — | — | — | — |
| Mod. | | | | | | | | |

### Example Illustrating Correct Operation of the Volume-Domain Projection and Volume Estimation Algorithm Using Real EEG Data

This example demonstrates the method of volume estimation of independent component analysis (ICA)- derived scalp topographies using real EEG data and that the measure of volume overlap of components provides an objective and anatomically meaningful metric of the physical volume separation of each component.

EEG data were collected from 31 participants while they navigated a virtual computer environment. The data for all participants and conditions were concatenated and then separated into components using ICA. The brain volumes for a selected set of components were calculated. The volume overlap of components and the distance between component centers of mass were calculated and examined in conjunction with topographical characteristics to determine the volume separation of each component.

Component volumes occupied brain regions involved in spatial navigation, working memory, and object recognition. The collective characteristics of distance between centers of mass and dipole characteristics of topographies indicate that, of the 9 components examined, all pertain to unique parts of the brain. The metric of volume overlap, however, indicates that there is a possible partial volume overlap for 2 pairs of components. This indicates that these components may not be fully associated with separate anatomical brain areas.

We conclude that our methodology successfully estimates the volumes of ICA-derived EEG components and that the separation of brain activities can be evaluated by a physical volume-domain measure.

Data from 31 participants were used. To provide enough data for a strong representation of brain activity, components for volume estimation were calculated from a single ICA decomposition of a grand EEG dataset. Each participant contributed a dataset of 29 trials, with 875 samples/trial, for each of the 2 behavioural conditions. To reduce the memory requirements to process the data, the sampling rate was reduced. The data of each trial were band-pass filtered from 8 Hz to 30 Hz using a 60-point zero-phase FIR filter and then down-sampled to 125 samples/second. Each of the datasets were concatenated to provide for a single grand dataset of 112 channels with 785726 samples (i.e. 29 trials×437 samples/trial×31 participants×2 conditions). The runica algorithm was used to compute component activities and topographies. The ‘logistic’ parameter was used for the EEG data decomposition. To compensate for the rank reduction of the data via the previous artefact removal step, we reduced the rank of the data to 70 components using the ‘PCA’ parameter. While the concatenation of datasets from multiple participants does weaken the assumption of spatial stationarity, our experience is that the effect of this is more than offset by the overall increase in the quantity of data.

Of the 70 components returned by runica, 9 components were selected to demonstrate the volume estimation methodology. The section criterion was of source ‘goodness’, based on the dipolar characteristics of source topographies (Onton and Makeig, 2006). A dipolar topography has ideally two foci and a smooth transition of variance away from each of the foci. A topography may also be considered as dipolar with one foci and the requisite smooth transition away from the foci, presuming that the orientation of the dipole places the second foci in the ventral direction were no electrodes are placed. The selected source topographies were prepared for projection to the volume domain by subtracting the mean from each topography (as was previously done in the step of average referencing the data) and were normalized using the Euclidean norm.

The raw volumetric spectra and the volumetric spectra for multiple thresholds were calculated using the volume estimation algorithm. This algorithm utilizes a combination of the LCMV Beamformer and statistical thresholding of an estimated volumetric spectrum to define modular source volumes from the continuous volumetric spectrum.

The volumetric spectra were first calculated for each topography. This algorithm utilizes the LCMV beamformer to project the variance of ICA-derived scalp topographies into a continuous representation of variance in head model volume. An idealized LCMV Beamformer input is created using topographies calculated via an ICA decomposition of EEG data. Idealized data created from each topography, were then individually projected into the volume domain using a bias-corrected version of the LCMV Beamformer. This provides for a spectrum of volume domain coefficients representing projected variance at all points in the head volume.

We then calculated modular volumes to depict the volumes of sources recovered in the ICA decomposition of EEG data. To do so, an estimate of the volume domain noise was used to define the edges of the volume-projected source. To calculate this noise estimate, it is assumed that the volumetric spectrum is largely representative of noise and that the volume of the source is represented by relatively few coefficients. The brain source volume is represented by outliers in the distribution of noise and the edges of the volume are identified by a threshold calculated as some number of standard deviations above the mean noise estimate (STDM). From the thresholded spectra, the modular volumes for each selected component are defined. For this study, those voxels within 4 and 5 STDM are defined by boundaries indicated by 3D concentric shells encapsulating each region.

To view the relationship between the threshold selected and the calculated source volume, the modular volumes for multiple thresholds was calculated. A summary of the volume estimates corresponding to a threshold range of 2 STDM to 6 STDM for each of the components examined was plotted.

To examine differences in the overlap estimate for the raw and the thresholded spectra of the modular volumes, the pair-wise volume overlap (PVO) was calculated for both cases. This was done using Equations 33 and 34for both the raw spectra (Table 1) and for the spectra thresholded at 4 STDM (Table 2).

First, each volumetric spectrum was mean subtracted and normalized using Equation 33,

$$\hat{r} = \frac{r - \overline{r}}{\sqrt{{( {r}_{1} - \overline{r} )}^{2} + {( {r}_{1} - \overline{r} )}^{2} + … + {( {r}_{1} - {\overline{r}}_{P} )}^{2}}} \qquad ( 33 )$$

where r is a 1×P vector containing the volumetric spectrum coefficients r₁, r₂, to rₚ, r̅ is the mean, and \hat{r} is the zero-mean, normalized spectrum.

The PVO was then calculated as a pair-wise scalar product as in Equation 34,

Sᵢⱼ=|(\hat{r}ᵢ\hat{r}ⱼ^{T})|for all i i≠j (34)

where Sᵢⱼ is the overlap for an arbitrary pair of zero-mean and normalized volumetric spectra \hat{r}ᵢ and \hat{r}ⱼ.

To calculate differences in the centers of mass between components, the following steps where used. First, the center of mass of each source was found as the head volume index location of the peak coefficient of each volumetric spectrum. Second, the distance between pairs of source centers dᵢⱼ was calculated for all pairs of components using Equation 35,

dᵢⱼ=\sqrt{(xᵢ−xⱼ)²+(yᵢ−yᵤ)²+(zᵢ−zⱼ)²} (35)

where xᵢ, yᵢ, zᵢ and xⱼ, yⱼ, zⱼ are the centers of mass of a pair of sources. The results were tabulated with corresponding measures of volume overlap for comparison.

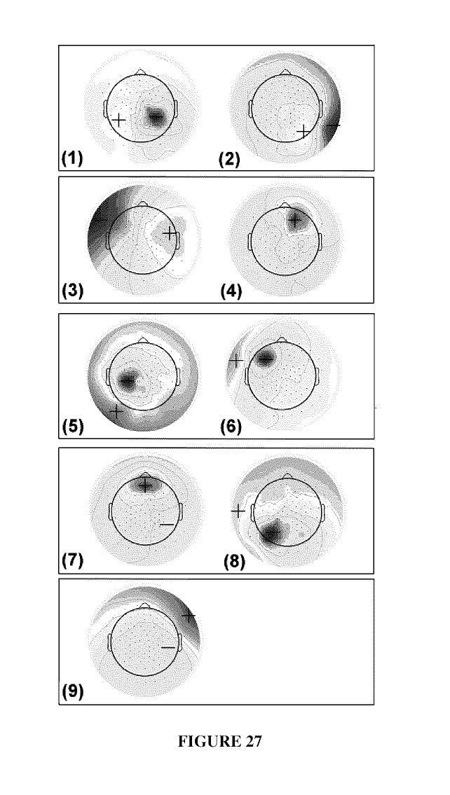

ICA decomposition of the EEG data yielded 70 components. Of these, the topographies of 9 components selected for volume estimation are plotted in FIG. 27 and labelled as (1) through (9).

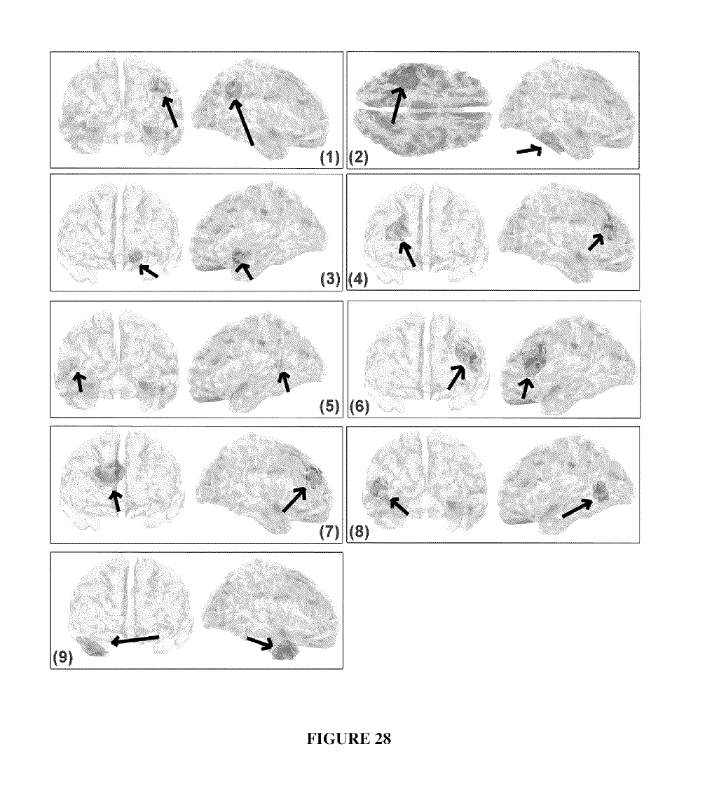

Source volumes were found to reside in multiple areas of the brain and are depicted in FIG. 28. FIG. 28 shows source localization and volume estimation results for brain sources (1) through (9) projected into a white-matter frame. The two views that best illustrate the source volume within the model head are given. STDM thresholds are indicated as colored shells; 4 STDM is indicated by regions of light-grey; 5 STDM is indicated by regions of dark-grey.

Two perspectives of each source volume provide axes for viewing volume size, shape, and location. Visual inspection reveals that the estimated source volumes are approximately located in the: (1) right parietal cortex, (2) right inferior posterior temporal cortex (or ventrotemporal cortex), (3) left medial orbitofrontal cortex, (4) right lateral superior frontal sulcus (or middle frontal gyrus), (5) left inferior posterior parietal cortex (or left angular gyrus), (6) left dorsolateral prefrontal cortex, (7) right medial superior frontal gyrus, (8) left posterior superior temporal gyrus, and (9) the right temporal pole.

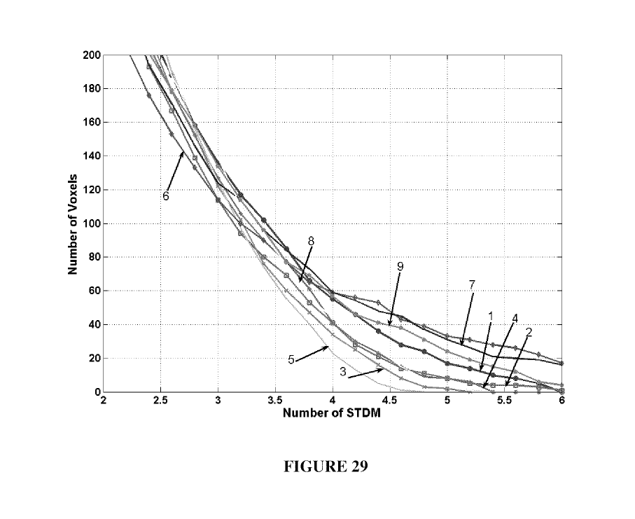

A plot illustrating the number of voxels occupied by each source at various STDM thresholds is provided in FIG. 29. FIG. 29 shows source volume estimates for multiple STDM thresholds plotted as the volume estimate (number of voxels) versus the threshold used (number of STDM). Each of the 9 brain sources examined is labelled using arrows as 1 through 9. Notably, all sources have similar characteristic curves relating the number of voxels as a function of STDM. Generally, the curves have a steep negative slope between 2 and 3.5 STDM that decreases when values are greater than 3.5 STDM.

The PVO of raw volumetric spectra and the absolute distance between the centers of mass of each source is given Table 6. The notably large PVOs are 0.881, 0.935, and 0.553 for source pairs (4)-(7), (5)-(8), and (2)-(9), respectively. The distance between centers of mass for these components (below each PVO given in Table 1), however, illustrates that these pairs of sources do not have the same centers of mass. The distances measured between source centers of these components are not precisely zero. Notably, source pair (5)-(8) has a source center separation of only 1.12 cm while source pairs (4)-(7) and (2)-(9) have a separation of 2.12 and 4.95 cm respectively.

TABLE 6
Raw source volume overlap (top row) and distance between centers of mass
(bottom row). Values greater than 0.5 are given in bold italics. Distance units
are centimetres. Distances less than 5 centimetres are given in standard bold.

| | (2) | (3) | (4) | (5) | (6) | (7) | (8) | (9) |
|---|---|---|---|---|---|---|---|---|
| (1) | 0.202 | 0.428 | 0.272 | 0.196 | 0.390 | 0.311 | 0.0693 | 0.271 |
| | 6.53 | 10.1 | 9.11 | 9.71 | 12.1 | 9.97 | 9.87 | 9.86 |
| (2) | — | 0.0235 | 0.0471 | 0.236 | 0.391 | 0.167 | 0.105 | 0.553 |
| | — | 8.57 | 10.2 | 11.0 | 13.4 | 11.4 | 10.9 | 4.95 |
| (3) | — | — | 0.152 | 0.0313 | 0.297 | 0.141 | 0.176 | 0.356 |
| | — | — | 7.37 | 7.78 | 5.89 | 6.80 | 8.38 | 6.78 |
| (4) | — | — | — | 0.464 | 0.0277 | 0.881 | 0.460 | 0.341 |
| | — | — | — | 12.4 | 8.63 | 2.12 | 13.2 | 8.56 |
| (5) | — | — | — | — | 0.0246 | 0.404 | 0.935 | 0.330 |
| | — | — | — | — | 7.57 | 11.6 | 1.12 | 12.5 |
| (6) | — | — | — | — | — | 0.131 | 0.0847 | 0.135 |
| | — | — | — | — | — | 6.82 | 8.63 | 12.36 |
| (7) | — | — | — | — | — | — | 0.421 | 0.199 |
| | — | — | — | — | — | — | 12.5 | 9.66 |
| (8) | — | — | — | — | — | — | — | 0.342 |
| | — | — | — | — | — | — | — | 12.7 |

The PVO for a threshold of 4 STDM (corresponding to the light-grey shells of FIG. 28) are given in Table 2. Notably, when noise in the volumetric spectrum is removed via thresholding, the overlap is still evident between source pairs (4)-(7) and (5)-(8), while there is no overlap between (2)-(9). In fact, the overlap between (2)-(9) for this 4 STDM threshold is less than the overlaps for (7)-(9) and (6)-(9) when no threshold is used.

TABLE 7
The volume overlap of pairs of sources thresholded at 4 STDM. Numbers in
bold italics correspond to comparisons highlighted in Table 6.

| | (2) | (3) | (4) | (5) | (6) | (7) | (8) | (9) |
|---|---|---|---|---|---|---|---|---|
| (1) | 0.00877 | 0.00799 | 0.00866 | 0.00657 | 0.0104 | 0.0105 | 0.00919 | 0.103 |
| (2) | — | 0.00690 | 0.00748 | 0.00567 | 0.00899 | 0.00903 | 0.00794 | 0.00893 |
| (3) | — | — | 0.00681 | 0.00516 | 0.00818 | 0.00822 | 0.00723 | 0.00813 |
| (4) | — | — | — | 0.00560 | 0.00887 | 0.297 | 0.00784 | 0.00882 |
| (5) | — | — | — | — | 0.00672 | 0.00676 | 0.337 | 0.00668 |
| (6) | — | — | — | — | — | 0.0107 | 0.00941 | 0.0106 |
| (7) | — | — | — | — | — | — | 0.00947 | 0.0106 |
| (8) | — | — | — | — | — | — | — | 0.00936 |

### Example Illustrating Correct Operation of the Volume-Domain Validation Algorithm

To determine whether the volume-domain characteristics of the runica method of ICA decomposition of EEG data can be used to differentiate components relating to brain activity from artefacts. To show ICA convergence characteristics provide confidence in component estimates.

Synthetic EEG data containing brain and noise sources were created. The mixture was separated using the runica, ICA algorithm. For each progressive iteration of reduced statistical dependence calculated via runica, scalp variance from component topographies was projected to the volume domain. Using these projections, the peak spectral value, average volume overlap and median volume overlap between estimated component volumes, and the distance travelled by component centers of mass were calculated. ICA components were then ranked using these measures calculated for the final iteration. This was repeated using real EEG data.

Ranking components according to these measures differentiated synthetic brain activity components from artefacts. Good volume representation was indicated by large peak spectral value while minimal volume overlap and component uniqueness was indicated by low median and average volume overlap. Progressive iterations improved these measures beyond the initial PCA step of runica. The distance travelled by components at each iteration indicated which components had late-converging or non-convergent centers of mass. Analysis of real EEG data yielded comparable results.

Volume-domain characteristics of components can be used to differentiate artefacts from sources originating from inside the head.

For the current analysis, we first determined if the algorithm would perform as expected on idealized data as it is an assumption that clear convergence of volume-domain properties will result over iterations of the runica algorithm; it isn't appropriate in this first step to complicate the analysis by modelling source activities that are not separable by ICA. This part of the study examined the convergence of volume-domain characteristics of simplified synthetic data to determine whether maximization of statistical independence among idealized brain source time-domain activities relates to improved voxel specificity of their volume representation, decreased volume overlap, and consistent, timely convergence of the centers of mass of these components and in contrast, that canonical artefacts do not share these characteristics. Using the volume-domain characteristics calculated on the final iterative step of the runica algorithm, in conjunction with the total distance travelled by the centers of mass of components over all iterations of the algorithm, the components were ranked and classified to determine if canonical artefacts can be separated from idealized brain sources.

Once the logical convergence of volume-domain characteristics for modular brain volumes was successfully demonstrated on idealized data, the algorithm was examined using real EEG data in the second part of this study. In the second part, real EEG data were examined to determine if the proposed validation methodology can be applied to data that are more typical of real EEG data having high noise levels, varied strengths of source representation in the data, sources do not perfectly satisfy the assumption of spatial modularity, and sources that do not perfectly satisfy ICA assumptions of non-Gaussianity and statistical independence.

The head model artwork and mathematical head model parameters used for this study was derived from the open-source BrainStorm software package. A total of 5439 voxels define the three-shell BERG head model with a voxel grid spacing of 0.5 cm There was no constraint placed on the projection of variance other than that variance can only be represented within the confines of the model head volume. Matlab 7 (Mathworks Inc.) and EEGLab 4.515 with the runica source separation algorithm were used to do all modelling, calculations, and plotting.

The runica algorithm converged after 316 iterations, yielding multiple components from the synthetic EEG mixture. The decomposition resulted in five components corresponding to the five simulated brain sources, one component relating to the electrode artefact, and two components resulting from the rank estimation error. Five of the 8 recovered components were identified as the 5 synthetic sources used to create the EEG data and have been numbered: 1, 2, 3, 5 and 6. The topographies and waveforms of these components, calculated for the last iterative step of the runica algorithm, closely matched the synthetic waveforms used in the construction of the data (not shown). By visual inspection of the scalp topographies of recovered components, the electrode artefact was identified and labelled as component number 4. The recovery of these components demonstrated the successful separation of them from the EEG mixture as was intended by design of the data. Visual inspection of the resulting topographies also revealed two rank estimation error artefacts; these were labelled as component numbers 7 and 8. The topographies of component numbers 7 and 8 appeared to be a mixture of the other sources comprising the data (not shown).

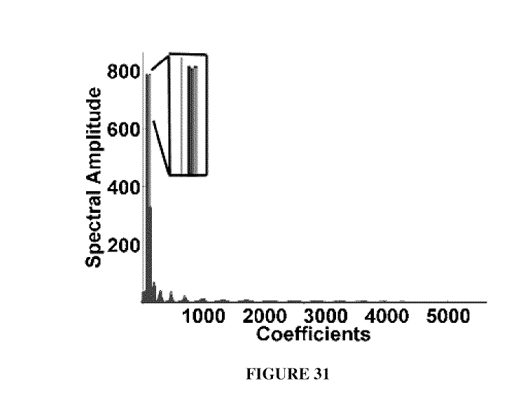

The volume-domain results corresponding to the calculated EEG components are plotted in FIGS. 30 to 31. The PSVs of each component corresponding to iterative step of the runica algorithm are plotted in FIG. 30a. The average and median PSV are given in FIG. 30b. The AVO and MVO are plotted in FIGS. 31a and 31b, respectively. The average AVO and median AVO are plotted in FIG. 31c. FIG. 31 shows the volumetric spectra of components 3 (dark-grey) and 4 (light-grey) superimposed to illustrate the concept of pair-wise overlap. Vertical axis is coefficient value. The horizontal axis is coefficient index. The more similar the spectra, the greater the calculated overlap ranging between 1 (maximum overlap) and 0 (minimum overlap).The inset magnifies the subtle differences in the spectra. The DT for each iteration is given in FIG. 32. The final PSV and TDT are plotted as FIGS. 33a and 33b while the final MVO and AVO are plotted as FIGS. 33c and 33d.

By the final iteration of the runica process of estimating maximally statistically independent components via the time-domain activities of the EEG mixture, the PSV of each of the 8 individual components had converged (see FIG. 30a). The final PSVs of the brain activity components (1, 2, 3, 5, and 6) are generally much larger than those for the electrode artefact (4) and rank estimate artefacts (7 and 8). The PSV convergence of the multi-voxel component (component 3), is notably smoother than the other components suggesting some fundamental difference between source types. This smoothness is an indication that ICA is sensitive to the voxel specificity of a brain source, since by design, the 26 voxel source is much wider and occupies more voxels than the single voxel sources. The PSVs of all components clearly exceeded the initial value (calculated in the PCA step) over multiple iterations with the exception of the electrode artefact, component (4).

The average and median PSV curves summarize the overall PSV convergence characteristics of all 8 components (see FIG. 30b). Both the average and median values are low for the first iteration and increase and stabilize at a higher value. Notably, the average shows a greater overshoot and instability than the median for early iterations as the average estimate reflects components (namely components 1 and 2) that have very large spectral peaks and some initial instability. The overshoot of component 2 plotted in FIG. 30a is clearly reflected in the average plotted in FIG. 30b. Comparing FIG. 30a with 30b, it is clear that the bona fide components provide the greatest PSV contribution that accounts for the overall average and median PSV improvement.

An illustration of the concept of pair-wise volume overlap is given in FIG. 31 showing why this metric is a continuous measure of volumetric similarity. The coefficients describing the projected volume-domain variance for components 5 and 6 (from topographies derived from the final iteration of the runica algorithm) have been plotted on the same figure to compare the relative values of the coefficients. Each coefficient represents the projected volume-domain variance (or spectral amplitude) at a unique location inside the head model. The volume overlap is a summary measure of how the values of coefficients differ at each grid location in the head model. The coefficients are ordered on a 1-dimensional axis according to the index position in the head model. If the two spectra in the figure were both identical, the volume overlap would be exactly 1. This would indicate perfect overlap of the volumetric spectra and that both volumetric spectra pertain to exactly the same brain volume.

The changes in the AVO and MVO over iterations reveal that the estimated overlap as a covariance of spectral coefficients of volume pairs decreases over runica iterations. The AVO and MVO of each component for each iterative step of the runica algorithm is given in FIGS. 32a and 32b, respectively. At the second iteration, the AVO of 4 of the 8 components (numbers 1, 6, 7, and 8) dramatically increase and then decrease to values less than the first iteration indicating an initial momentary instability but overall improvement in this volume overlap estimate. The AVO of the remaining components (2, 3, 4, and 5) decreases from the first iteration to the second and generally continues to improve thereafter. The MVO reveals a similar pattern for all components; after the second iteration the MVO for each component decreases. This general decrease shows multiple local peaks and irregularities to approximately 200 iterations at which point the AVO and MVO stabilize.

The average and median AVO for all 8 components are given in FIG. 32c. As with the AVO and MVO for each component separately, the average and median AVO show the initial increase in the estimate of volume overlap on iteration number 2 and that these values decrease quickly towards convergence at a low estimate of overlap. This indicates that when the values converge, the sources are unique by modular volume and therefore well separated. During convergence, however, local peaks and irregularities continue for about 200 iterations and then stabilize. The final average and median AVO are lower than those values calculated for the first iteration, indicating that ICA separation of these sources reduced the effective volume overlap of components.

FIG. 33 illustrates the instability of the rank estimate error components (components 7 and 8). The main plot of the figure illustrates the distance travelled by the centers of mass of the rank error components for each runica iteration. The continuous movement of the center of mass of these components beyond the 200^{th} iteration , and up to approximately iteration 300, is indicated by the arrow (g) in the figure. For comparison, the distance travelled for each iteration of the more stable bona fide components in the data are indicated by arrow (a) showing convergence before the 15^{th} iteration. Notably, the centers of mass of these components never travel more than 2 cm on an iteration and are represented only in the bottom left-hand corner of the figure. On a single iteration, the rank estimate error artefacts, however, moved distances greater than 7 cm—greater than half the width of the brain. The inset of FIG. 33 provides a 3-dimensional depiction of the travel of the centers of the mass of the rank-error estimate components, displaying their spatial variation (indicated by arrow (c)). This ‘spider web’ of lines showing the path of the centers of mass between iterations clearly shows the unreliability of these components. Their centers of mass do not simply reverberate around a single location, but travel around most of the right hemisphere. The centers of mass of bona fide components (1, 2, 3, 4, 5, and 6) clearly have different characteristics of travel. They move a very short distance (as indicated by the main figure window) and are thus minimally represented in the 3-dimensional plot of the figure inset. The summary statistic, TDT captures the extent of travel of these components for the entire minimization process.

The starting location of each center of mass is indicated as a ‘+’ in the inset of FIG. 33. The final locations are indicated in the figure inset as (b) for the three proximal simulated brain sources (f) for the single 26 voxel brain source, (d) for the distal brain source. The electrode artefact component (4) did not travel after the first PCA iteration and has zero travel distance, indicated in the figure inset as (e).

### Component Ranking by Volume Characteristics

Each of the 8 components are ranked in FIG. 34 on the basis of their final PSV, AVO, MVO, and TDT scores to illustrate how relative scoring by each measure separates artefacts from the simulated brain sources. For each figure, the values have been sorted and plotted from left to right such that the worst components are on the left side and the best components are on the right side of each plot. Where possible for each measure, a line dividing brain activity components from artefacts indicates that the measure separated artefacts (or a class of artefact) from other components. The ranked PSVs are given in FIG. 34a, with a separation of the ‘rank estimate artefacts’ (7) and (8) and the electrode artefact (4) from the simulated neural components. Similarly in FIG. 34c, the MVO clearly separates artefact components (7), (8), and (4) from the simulated neural components. FIG. 34b illustrates clear separation of the ‘rank estimate artefacts (7) and (8) from the other components using the measure of TDT. Lastly, FIG. 34d shows that artefact and simulated neural source components were not separated using the AVO.

### Real EEG Data

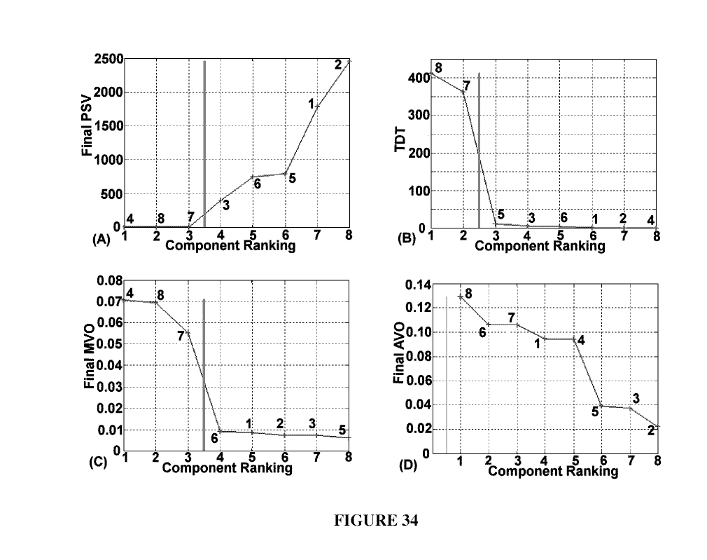

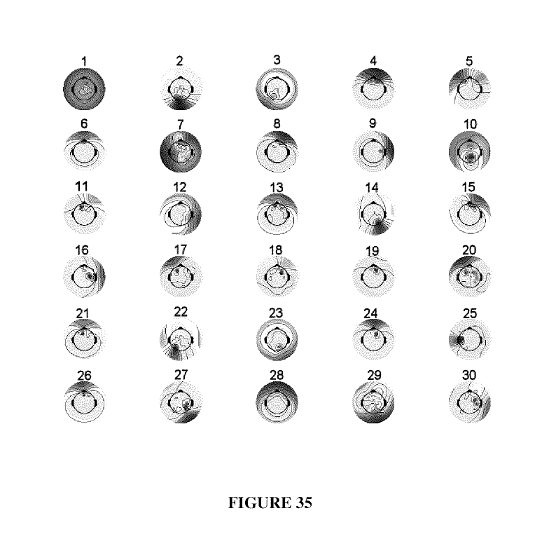

The runica decomposition and following component validation process followed steps similar to the analysis of the synthetic EEG data. The runica decomposition of real EEG data automatically converged and exited after 488 iterations yielding 30 components with corresponding waveforms and topographies. The topographies of components calculated for the final iteration are plotted in FIG. 35 for visual inspection. A subset of these components likely corresponds to good representations of brain activities originating from modular areas of the brain while others are artefact, non-modular brain activities that do not pertain to a specific area of the brain, or are poorly separated brain activities.

### Volume Domain Convergence

The average and median PSV convergence curves plotted in FIG. 36a show that, runica processing of real EEG data, generally improves the volume-domain specificity of components. Essentially, over sequential steps of the runica iterative process, the volume representations of components become more focal towards a single voxel, and specific to a particular region of the head. By iteration 100, the median PSV plateaus; subsequent iterations actually decreases the median value slightly suggesting possible overtraining of the ICA weight separation matrix and iteration of the runica algorithm on noise. The average PSV from iteration 100 to 488 is visually flat. The median and average PSV of components clearly exceeded the initial value (calculated in the PCA step) over multiple iterations. The offset difference between the median and the average (the average has a large positive offset compared to the median) suggests that the PSV improved by a large amount for a small number of components while for most components, the PSV improved only by a small amount, decreased, or did not change.

The changes in the average and median AVO and MVO given in FIG. 36b suggest that the measure of volume overlap by how the coefficients of the covary as a surface using the current AVO and MVO measures is unstable for real data. This instability is evident as an initial spike for both the MVO and the AVO corresponding to early iterations (similar to the case for the synthetic data), however after the spike the measures only decrease by a small amount and then slowly increase as iterations continue. After iteration 100 (for which the PSV converged), the average and median MVO and AVO begins to diverge and increase; the average and median measures do not converge for the real data as it did for the synthetic data.

The changes in the average and median AVO′ and MVO′ (which make volume overlap comparisons analogous to comparing the direction of vectors) of FIG. 36c reveal that this measure of estimated overlap generally decreases over runica iterations and is a stable measure. By iteration 100 these plots appear to be nearly converged on minimum values. The median and average plot of the MVO' demonstrates a slight increase between iteration 100 and 488 suggesting possible overtraining.

Examining the AVO and MVO of individual components in relation to the average plot of FIG. 36b reveals that the AVO' and MVO measure is indeed unstable for real data (results not shown). Many of the individual components have AVO and MVO values that change from decreasing to increasing after iteration 50 while others change between decreasing to increasing multiple times prior to completion of the runica algorithm indicating that these measure are not a good property to describe the volume overlap and volume uniqueness characteristics of an individual component.

Examining the PSV, AVO′, MVO′ and DT for components individually (FIG. 37) shows improved volume-domain characteristics over the runica estimation process for a select subset of components. Notably for approximately 15 components, the PSV (FIG. 37a) increased as expected while the MVO′ and AVO′ (FIGS. 37d and 37d, respectively) decreased as expected. Interestingly, the MVO′ of all of the components started at approximately 1 indicating that after the PCA step of the runica algorithm, there was considerable overlap among all components of the decomposition. By iteration 100 of the runica process most components appear to have converged according to the PSV, MVO′, and AVO′ plots; this is with the exception of component 20 which appears to be approaching convergence. Up to iteration 100, these volume-domain characteristics have improved for at least 15 components. After approximately iteration 100, the PSV, MVO′, and AVO′ of a subset of these components experience small reductions in improvement suggesting over-learning. The DT for each iteration illustrated in FIG. 37c shows that the centers of mass of most components converged prior to iteration 50. However, the centers of mass for 8 components continued to make small movements (steps less than 3 cm) between iteration 100 and iteration 450. These later movements occurred for component numbers 10, 17, 20, 29, 7, 13, 21, and 11.

Each of the 30 components are ranked in FIG. 38 on the basis of their final PSV (FIG. 38a), AVO′ (FIG. 38b), MOV′ (also FIG. 38b), along with the TDT (FIG. 38c) over iterations of runica to determine which of these components could be representative of modular brain sources. In each figure, the sorted values are plotted from left to right such that the worst components are on the left and the best components are on the right. Where appropriate, a vertical line indicating the knee of the curve has been placed to divide the left and right plot areas. The relative PSV scores of FIG. 38a suggest that components 10, 23, 28, 3, 20, 12, 1, 4, 5, 7, 14, 22, 29, 2, 27, and 16 might be components originating from the head that could represent brain activities of varying qualities. The remaining components 15, 13, 19, 11, 18, 8, 24, 26, 9, 21, 17, 30, 6, 25 are allocated as artefacts. The relative MVO′ and AVO′ scores plotted in FIG. 38b are very similar each other and their ranking also have some similarity to the ranked PSV scores. The MVO′ and AVO′ scores suggest that components 2, 14, 22, 29, 7, 5, 23, 1, 12, 3, 4, 20, 28, 10 are possibly components that originate from inside the head and have varying degrees of 3-dimenstional volume overlap. The MVO′ and AVO′ scores indicate that components 30, 9, 18, 24, 15, 19, 13, 8, 11, 26, 6, 21, 17, 27, 16, and 25 are artefacts. The TDT plot of FIG. 38b does not have a characteristic curve from which a knee can be selected to separate possible artefacts from sources originating from inside the head. Instead it provides a relative ranking of the stability of each of the components where components 17, 18, 15, 1, 7, 19, 5, 3, 25, 27, 10, 9, 8, 26, 23, 2, and 6 are most stable and components 20, 24, 30, 29, 16, 13, 11, 14, 28, 12, 4, 21, and 22 are less stable. Taken together, the AVO′, MVO′ and PSV plots indicate that approximately half of the components of the decomposition can automatically be regarded as artefacts and can thus be automatically rejected from further analysis.

The ranked scores for the PSV, MVO′, AVO′, and TDT are given in Table 8 such that the best components according to each measure are on the right side of the table while the worst components according to each measure are on the left side of the table.

TABLE 8
Component scores for PSV, MVO, AVO, and TDT sorted from worst to
best from left to right. For PSV, the largest PSV corresponds to the
component on the right side of the table while the lowest PSV corresponds
to the component on the left side of the table. For MVO and AVO, the
largest value corresponds to the component on the left side of the table
while the right side of the table contains the component corresponding
to the smallest value. The component with the largest TDT is given on the
left side of the table while the right side of the table contains the
component with the smallest TDT.

| | |
|---|---|
| PSV | 15, 13, 19, 11, 18, 8, 24, 26, 9, 21, 17, 30, 6, 25, 16, 27, 2, 29, 22, |
| | 14, 7, 5, 4, 1, 12, 20, 3, 28, 23, 10 |
| MVO | 30, 9, 18, 24, 15, 19, 13, 8, 11, 26, 6, 21, 17, 27, 16, 25, 2, 14, 22, |
| | 29, 7, 5, 23, 1, 12, 3, 4, 20, 28, 10 |
| AVO | 9, 18, 15, 24, 26, 11, 19, 13, 8, 6, 21, 30, 17, 25, 27, 16, 2, 14, 29, |
| | 22, 7, 5, 23, 1, 12, 3, 4, 20, 28, 10 |
| TDT | 20, 24, 30, 29, 16, 13, 11, 14, 28, 12, 4, 21, 22, 17, 18, 15, 1, 7, |
| | 19, 5, 3, 25, 27, 10, 9, 8, 26, 23, 2, 6 |

The results of expert validation of components by visual inspection of their topographies of FIG. 35 are tabulated in Table 9. FIG. 35 shows topographies of components calculated from real EEG data that were returned by runica source separation. Each topography has been assigned a unique number identifier. This illustrates the necessity to identify good components as there are a large number of components and it is difficult to decide which ones to keep for analysis. Components are allocated as either ‘E’ for eye-artefact, ‘B’ for good brain activity that might originate from a single modular brain location, ‘b’ for a poorly separated brain activity, and ‘-’ indicating that the component is clearly an artefact. Components that are ambiguous are tagged with a ‘u’.

Also provided in Table 9 are the ranking of components by the PSV and MVO′ scoring process is given in Table 8 to compare the automated and expert ranking results. The comparisons shows that the MVO′ and the PSV are complimentary; if either of the MVO′ or the PSV find a component as an artefact, then that component should be allocated as such. Those components listed as artefacts by the proposed validation method are in agreement with the expert analysis with the exception of components 7 and 27. Comparing the expert allocation of artefacts as eye-artefacts with the proposed validation method indicates that the validation method does not identify eye-movement or blink contamination of the EEG as artefact.

TABLE 9
Rating of components calculated from the real EEG dataset
for the PSV, MVO, and by expert evaluation.

| cnum | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| expert | B | B | B | E | E | — | — | — | — | B | — | B | — | B | — |
| PSV | H | h | H | H | H | — | H | — | — | H | — | H | — | h | — |
| MVO | V | v | V | V | V | — | V | — | — | V | — | V | — | v | — |
| Cnum | 16 | 17 | 18 | 19 | 20 | 21 | 22 | 23 | 24 | 25 | 26 | 27 | 28 | 29 | 30 |
| expert | — | — | — | — | b | — | B | B | — | — | — | B | E | b | — |
| PSV | h | — | — | — | H | — | h | H | — | — | — | H | H | h | — |
| MVO | — | — | — | — | V | — | v | V | — | — | — | — | V | v | — |

‘enum’ indicates the component number corresponding to FIG. 34.
The rating types for expert validation are: ‘B’ = good distinct modular brain activity; ‘b’ = poorly separated brain activity; ‘—’ = artefact or very badly separated brain activity; ‘E’ = eye-movement or blink related component; ‘u’ indicates the expert was uncertain.
The rating types for validation by PSV are: ‘H’ = good head model representation and voxel specificity; ‘h’ = moderate head model representation and voxel specificity; ‘—’ = poor head model representation and voxel specificity indicating an artefact.
The rating types for validation by MVO are: ‘V’ = good volume uniqueness (minimum overlap); ‘v’ = moderate volume uniqueness (moderate overlap); ‘—’ = poor volume uniqueness indicating an artefact.

### Example Illustrating Correct Operation of Algorithms for a Single-Participant Dataset

This example illustrates use of the data mining, volume-domain projection and volume thresholding components of the disclosed technology to process data from an individual participant.

This example shows that brain activity can be represented as functional nodes, with individual activities and correlated relationships. (See FIGS. 39 to 41). Importantly, these figures provide an easily interpretable and meaningful representation of brain activity at the system-level. These are not plots of uncorrelated or statistically independent components of the EEG, but rather they are plots of the estimated bona fide activities of specific brain volumes and their corresponding time-domain activation relationships determined using the Spectral Shaping ICA algorithm. The study in which these data were collected examined to behavioural conditions.

FIG. 39 illustrates multiple views of a set of 8 source volumes. These volumes represent the complete set of brain areas that were active during the data recording while the participant navigated the vMWT. The brain volumes resolved from these data are presented with respect to a canonical representation of the white matter of the brain. Modeled active brain volumes represent grey matter volumes while the white matter framework provides a physical frame of reference so that the resolved volumes can be named anatomically. A close-up of the brain volumes of the ventral visual pathway is labelled in FIG. 39d. The relative locations of these volumes on the white matter cortex suggests they occupy the inferior portions of Brodmann areas 17, 18, and 19, bilaterally, and area 20 on the right side of the brain. A volume is also represented in the dorsolateral region of the left hemisphere frontal lobe, possibly the hand area of the motor homunculus. There is also a volume near the midline posterior to the cingulate cortex of the right hemisphere, possibly Brodmann area 30 or 31. The appearance of these areas in the data indicates that the participant was actively using their ventral visual pathways during the task. This finding is consistent with the maze navigation behaviour of the paradigm as there is a requirement for visual discrimination and identification of features in the vMWT. The source separation and volume estimation methodology appear to have provided anatomically meaningful results.

The volumes identified that define the ventral visual pathways appear to reflect the cyto-architectonics of the cortex. Having done so has allowed the labelling of each volume by a different Brodmann number. Each part of the visual pathway is known to provide a different type of function in processing visual information, and because the data-mining algorithm identified them as separate parts, it is apparent that the relationship between activities of these parts changes over the duration of the task. These parts differ by cellular characteristics and thus likely have different electro-capacitive and conductive properties. It is reasonable to conclude that these properties facilitate the source separation algorithm to identify them as separate components. (In fact, this is shown by differences in the brain sources waveform activities compared in the power plot of FIG. 40 shown later.)

Information describing which areas of the brain are active, when they are active during the task, and under what behavioural conditions they are active is provided via time-varying power levels that correspond to each of the resolved brain volumes. For example, the plots of FIG. 40 provide the trial-average time-varying power-levels of brain volumes identified in the left and right ventral visual pathways. These have been calculated from the source time-varying waveforms corresponding to each brain area. To do so, the time-domain samples of each waveform of each trial and condition were squared to calculate instantaneous power. The power waveforms were then ensemble averaged to provide a generalized representation of the stimulus-locked power levels of each brain volume corresponding to each brain area for each behavioural condition. It is possible to make power level comparisons among behavioural conditions as the same scaling values are used for the data from both conditions. Waveform shapes can be compared for all cases and the changes in the power levels of these areas over time are meaningful.

The plots given in FIG. 40 reveal differences in the trial-averaged activation power levels for the cue and place behavioural conditions. The Left hemisphere activities for cue and place are given in FIGS. 40a and 40c respectively (left side of the plot) while the right hemisphere activities for cue and place are given in FIGS. 40b and 40c respectively (right side of plot). The colors used in these plots correspond to the colors of FIG. 39 that illustrate each modelled brain volume. One notable difference between the activation power levels for cue and place occurs in the left hemisphere in Brodmann area 18 where the trial-average stimulus-locked activation level varies in amplitude with respect to time in a different way for each condition. Another notable difference is that for the cue condition, there is greater average power originating from Brodmann area 20 than for the place condition for the right hemisphere after approximately 75 ms after trial onset and persists after 100 ms. As well, there are differences for the earlier visual areas (Brodmann areas 17 and 18) at approximately 100 ms for the left hemisphere.

FIG. 41 shows time-varying pair-wise zero-lag correlations for two separate brain volumes and for the two behavioural conditions (place and cue) for the frequency band of ocular vergence. To create these representations, the activities of each source waveform for each trial of data for both behavioural conditions were band-pass filtered from 34 to 36 Hz. Once the activities in the frequency band of vergence were isolated, the time-varying correlation was calculated for each trial and each pair of components. The calculation of time-varying correlation used a 500 ms sliding window with 50% overlap. Prior to calculating correlation values for each window, each portion of the waveform was Hanning windowed to reduce edge effects on the correlation estimate. This created correlation values at steps in time for each trial and for each behavioural condition. These values were ensemble averaged for each of the cue and place conditions to create an average correlation estimate. Finally, the absolute values of these average correlation values were calculated. Error bars describing the variability of the correlation estimate across trials were calculated as the mean of random samples of the trials for each condition. There were 2000 draws of 20 random samples from the set of trials for each condition to generate this statistic.

The average stimulus-locked correlation estimates show clear differences between the behavioural conditions as a function of time. FIG. 41a represents the time-varying relationship between visual Brodmann area 18 of the right hemisphere and part of the motor homunculus of inferior Brodmann area 4 of the left hemisphere. FIG. 41b represents the time-varying relationship of Brodmann area 18 of the left and right hemispheres. These values are given for each of the two behavioural conditions, place (plotted in red) and cue (plotted in blue). Divergence in the error bars of two compared conditions or two intervals of time suggests that the link and level of cooperation for these conditions or time intervals differ. Thus, for this particular study participant, there is a relationship between the dorsolateral region of the left hemisphere frontal lobe and the visual area of the right hemisphere that differs between behavioural conditions shortly after the trial onset. As well, there is a clear weakening of the correlative relationship of activities between the two visual Brodmann areas 18 bilaterally after the trial onset.

### Example Illustrating Correct Operation of Algorithms in a Study of Spatial Navigation

This example demonstrates the feasibility of using a 32-channel scalp-EEG acquisition system to examine brain function related to spatial navigation using the disclosed technology. To construct a model of the functional relationship of brain areas active in egocentric (cue based) and allocentric (place based) navigation conditions during a virtual Morris Water Task (vMWT). To identify analysis methods that might be used in a larger study to analyze brain activities for neurodiagnostic purposes.

EEG data were collected while participants completed trials of a vMWT paradigm that were biased towards allocentric and egocentric navigation strategies. These data were separated into components using the Spectral Shaping ICA (SS-ICA) algorithm, validated using a volume-domain validation algorithm, and localized to examine brain activities associated with task conditions. The volumetric origins of activities of validated EEG components were estimated and depicted on a canonical cortex. Component activation comparisons were made between navigation conditions for the first second of navigation. Brain-behaviour relationships were identified by calculating correlations of component activation with trial completion latencies, and component activation with explicit knowledge of platform location. Pair-wise zero-lag correlations among component activities were calculated to estimate functional relationships between brain areas. Correlations were also calculated to determine the consistency of pair-wise activation of components across participants.

The EEG data were separated into 31 components. A subset of these components were linked to specific areas of the brain: the superior medial parietal lobule, primary motor, posterior parietal, anterior parietal, medial anterior parietal areas of the right hemisphere, the dorsal extrastriate visual, and the posterior inferior temporal cortex of the left hemisphere; bilaterally: the dorsolateral prefrontal cortices, superior parietal lobules, ventral extrastriate visual cortices, and primary striate visual cortices. Activities of the right hemisphere posterior parietal cortex were significantly greater during allocentric trials than egocentric trials. Activation of the ventral extrastriate visual cortices bilaterally and the dorsolateral prefrontal cortex of the right hemisphere were related to increased average time to complete trials optimized for the allocentric navigation condition. No significant correlations of brain area activation with accuracy of explicit knowledge of the platform location were found. A greater number of zero-lag correlations among multiple brain areas were found for the allocentric condition than the egocentric condition.

It is feasible to use a 32-channel scalp-EEG acquisition system to examine brain function related to spatial navigation using the disclosed technology. The disclosed technology was used to successfully model the functional relationship of brain areas active in egocentric (cue based) and allocentric (place based) navigation conditions during a virtual Morris Water Task (vMWT). Analysis methods were identified that might be used in a larger study to analyze brain activities for neurodiagnostic purposes.

The Spectral Shaping ICA (SS-ICA) EEG data mining process, a component of the MCST algorithm, was used to identify components of the EEG dataset as waveform activities and topographies. The SS-ICA method differs from standard ICA methods such as runica because the source separation criterion of statistical independence is not applied equally at all frequencies; a subset of frequencies is permitted to be correlated and dependent. To do so combines characteristics of the BS-ICA algorithm and the runica algorithm. The improvement provided by the SS-ICA method over runica has been demonstrated in prior work.

The SS-ICA algorithm automatically calculates an appropriate filter by which to shape the EEG frequency spectrum and calculate spatial separation matrices. The shaping filter characteristics are determined by identifying frequencies of statistical independence that provide reduction of the overall dependence measured among EEG components. The magnitude spectrum of this filter indicates which frequencies have the greatest independence and which frequencies have the least independence. We have found that the frequencies less than 20 Hz and in the interval 45-55 Hz had the least independence, while the frequencies in the intervals 20-45 Hz and 60-75 Hz had the greatest independence. Once the coefficients of this filter were calculated, they were used to shape the prepared EEG data, emphasizing frequencies of independence and uncorrelatedness by which to calculate spatial separation matrices. The spatial separation matrices were then used to separate the original unshaped EEG data into components and determine the topographical characteristics of these components.

To characterize each component by its volume-domain properties for subsequent steps of component validation and brain source volume estimation, the volumetric spectrum of each component was calculated. The volumetric spectrum is a 3-dimensional volume-domain representation of the topographical scalp surface field and is calculated via a process of projecting the topographical characteristics of each component into a mathematically defined head model. This process maps the spatial variance measured at the scalp surface, described by each component topography to a volume-domain representation using a closed-form mathematical solution. In the current study, a three-shell head model, created using the BrainStorm software package, comprised of 5588 voxels, was used to describe the variance at every location in the head model. Each voxel was defined as cube with the length of each side equal to 5 mm. The levels of projected variance at each of these voxels, when arranged as a 1×5588 vector, defined the volumetric spectrum.

Components calculated via the data mining process were validated using two methods, first by physical modeling of component characteristics in the volume-domain, and second, by waveform frequency characteristics.

The first validation method using physical modeling of the volume-domain characteristics of components, rated components according to how well they could be represented as volumes within a model head. Each component was rated according to two properties: the specificity of representation of components at a single voxel in the head and the volume-domain overlap of each component with other components of the decomposition. The voxel specificity property is determined as the value of the largest coefficient that defines the volumetric spectrum of each component and is herein referred to as the peak spectral value (PSV). The second property, the volume overlap of components, was calculated via a 2-step procedure. This procedure essentially estimates the uniqueness of the volumetric spectra of components. First the pair-wise overlap of the volumetric spectrum of the component under evaluation with the volumetric spectra of all other components of the decomposition is estimated. Each pair-wise overlap estimate is calculated as the Euclidean normalized dot product of the pair of volumetric spectra. Second, to provide a summary of overlap for the component under evaluation, the median of the dot products calculated in the previous step is calculated and is herein referred to as the median volume overlap (MVO) of that component. In the current study, the PSV and MVO were determined for each component of the decomposition. To assign a rank to each component by which to identify possible ‘brain’ components from ‘artefact’ components of the decomposition, the PSV and MVO values calculated for each component were sorted and compared against the values of all other components. The distinction between possible ‘brain’ component representations and ‘artefact’ component representations was determined by defining a threshold at the knee of the curve of these sorted PSV and MVO values. Those components that were ranked just below the knee of the curve were noted as ‘uncertain’.

Since the volume-domain validation procedure described above is relatively new, a second validation method examining component waveform frequency characteristics was used to corroborate the results of the first method. Using the Welch method, the frequency spectrum of each component was estimated. Visual inspection and evaluation of the frequency characteristics of components was and used to classify each component as containing brain activities or artefact activities. Those components with frequency spectra similar to the standard scalp EEG spectrum were noted as ‘brain’ and those with frequency spectra that are unlike a standard scalp EEG brain activity spectrum were noted as ‘artefact’. Those components with frequency spectra that were not easily distinguished as brain or artefact (or were a mixture of both) were noted as ‘uncertain’.

While validation by frequency characteristics does indicate which components have spectra that are ‘brain-like’, it does not provide information indicating how well each component pertains to unique anatomical areas of the brain (i.e., how well brain activities are separated). The result of the volume-domain criterion based validation of components was compared to validation of components by visual inspection of their frequency content.

Components of the EEG calculated via data mining were linked to anatomy by estimating the physical volumes inside the head from which the waveforms of components originate. These volumes were determined by estimating and removing the noise in the volumetric spectra calculated for each individual component. The variance contained in the volumetric spectrum that is not accounted for by the noise estimate is assumed to relate to the actual brain source. Noise thresholds were determined for both 4 and 5 standard deviations above the mean (STDM) noise-level. Prior work using simulated and real EEG data has shown that 4 or 5 STDM provides a reasonable depiction of the brain volumes from which component activities originate. Plots were created depicting the volumes occupied by validated brain sources. For completeness, the volumes of components that were not validated as having good volume-domain representations (those that were previously scored as ‘uncertain’ or ‘artefact’) were also plotted (not provided).

While component locations do provide information describing which areas of the brain have activities relating to the paradigm, this component location information does not differentiate the activities of each condition, cue versus place. Hence, the waveform activities of each component were examined to make comparisons among the cue and place conditions.

The remainder of the analysis in the current work used only validated components to examine brain function associated with this spatial navigation paradigm. Component waveform activities and behavioural data obtained during data collection provided evaluation of the conditions of the paradigm.

To EEG and behavioural data, statistics were calculated using both non-standardized and standardized data. By making both types of comparisons, it was possible to determine if there was a significant difference between the cue and place conditions, while including and excluding between-participant variability. Doing so illustrates how comparisons might be made in a larger experiment investigating classification of brain activities as either egocentric or allocentric.

The average latency to trial completion for the last half of each of the cue and place blocks was calculated for each participant and plotted. To identify differences between the conditions for the group while including between-participant variance, an ANOVA was used on the non-standardized group latencies. To identify any effect of condition on latencies that includes between participant variance, the standardized latencies were used in an ANOVA to test for a significant effect. For comparison purposes, the standardized average and non-standardized average latencies were plotted.

Scores from the DS-probe trials were used as a measure to indicate the explicit knowledge of participants for the correct location of the hidden platform for the cue and place trials. A score of 1 was assigned if the participant placed the seed in the correct quadrant of the maze, and increasing with accuracy to the center of the platform to a maximum score of 7. A score of 0 was assigned if the participant did not place the seed in the correct quadrant of the maze. The scores for each participant were individually plotted for the cue and place conditions and were used to identify correlative relationships with the activities of components of the EEG. A matched-pair permutation test was applied to the DS-probe data to identify differences between conditions for the group. The matched-pair permutation test does not require that the data fit a Gaussian or Normal distribution and was applied because the DS-probe scores were found to be highly skewed. The test accounts for within-participant characteristics of the paradigm by maintaining the matched-pair characteristics of the cue-place data. This test assumes that the data acquired in both the cue and place condition are from the same distribution and identifies the probability of the observed mean difference between the cue and place conditions actually occurring. If the measured mean difference is unlikely to occur (p<0.05), then the DS-probe scores for the cue and place conditions are considered to be from different distributions. The average of the cue and place groups were plotted for inspection. Confidence intervals were not calculated because they are known to be unreliable when data are skewed and the sample is small (n=12).

To examine brain activations of the first second of navigation in the last half of each block of trials for the cue and place conditions, the 8-30 Hz root-mean-square (RMS) activities of each component were computed. First, component waveforms were band-pass filtered 8-30 Hz separately for each trial (trials 6 to 10) and condition using a zero-phase 300-point FIR band-pass filter. Next, an epoch of samples corresponding to the first second of navigation (0 ms to 1000 ms) was selected from each trial. These samples were then squared to provide an estimate of instantaneous power. The square-root of the mean of the squared samples was calculated to yield the RMS activation for each epoch. RMS activations of the last half of each block were averaged together to compute the average RMS activation for that condition. This was repeated separately for each participant, component, and condition. These activations for each component were plotted for each condition.

Standardized RMS activation values for each component and participant were calculated from the RMS activations determined above.

To determine if there was a difference between conditions for activities of each brain area, the data were evaluated using both standardized and non-standardized RMS activations. These were compared between cue and place conditions using an AVOVA. A comparison of the RMS activations for each component by condition for both the standardized and non-standardized data was plotted.

Brain area RMS activations were compared to latencies to identify possible relationships between brain activities in the first second of navigation and navigation performance in cue and place trials. Correlation values were computed between the RMS activation of each component and average latency to reach the platform across participants for the last half of each block for the place and cue conditions separately. The correlative relationships for each component and condition were plotted.

Correlations between RMS activation values of components were calculated in a standard correlation matrix for each condition separately. Pair-wise relationships were determined as the correlation of each component's RMS activation with other component RMS activations, across the participant group. These paired RMS activations were calculated to identify consistencies across the participant group in the pair-wise activation of components. These calculations were made separately for each condition using the pre-calculated non-standardized RMS activations. This was calculated for all possible pair combinations of components for each condition separately. Scatter plots were created to illustrate the pair-wise relationships between components so that the position of each participant on the plot served to identify trends and outliers in the data.

A block diagram illustrating significant pair-wise relationships indicating consistency across the group, calculated for each condition overlaid on anatomical information was created. Two ranges of significant correlation were provided in the figure to show a range of confidence (the roll-off of values from most confident). These pair-wise relationships were compared to significant relationships between behavioural latency and RMS activation as well as relationships between RMS activation and measures of explicit knowledge of platform location were included in the diagram.

The pair-wise zero-lag correlation between components was calculated to identify which brain areas have coordinated activities separately for each participant. This was accomplished through multiple steps using trials 6 to 10 for the cue and place conditions separately. First the data of each trial were band pass filtered 8-30 Hz for each brain activity component. Second, for each pair of brain activity components, a 50% overlap sliding analysis window 500 ms wide was used to estimate the correlation between the components for multiple time intervals during each trial.

For each analysis window the data were multiplied with a Hanning window to reduce spectral leakage. Doing so reduces distortion of the data caused by processing only a segment of the continuously recorded data. The windowed data were then normalized to have unit autocorrelation, prior to estimating the cross-correlation between the activities of component pairs. The cross-correlation value was then transformed using the atanh function to map the correlation sample distribution to a distribution closer to Gaussian. After calculating the correlated activities of all pairs at all time intervals, for each participant and condition, the correlated activities were plotted. A sample zero-lag correlation measured between pairs of brain area components that differ between behavioural conditions is provided in the Results section. Error bars were calculated as 1 standard deviation of random samples of the mean (2000 random samples of the mean; to calculated each mean, 4 participants were randomly selected from the group).

To summarize the pair-wise zero-lag results for the time-window around the onset of navigation, the above chance (p<0.05) zero-lag correlations that differed significantly between conditions were indicated by connecting lines on a model cortex.

The first step of the data mining procedure yielded a shaping filter implicating frequencies of relative independence. The shape of the magnitude spectrum of the shaping filter (automatically calculated) identified frequencies of independence in the band 25-45 Hz and frequencies around 70 Hz. Frequencies in the 34-64 Hz band and frequencies less than 25 Hz were de-emphasized suggesting the activities in these regions of the frequency spectrum have low statistical independence.

Upon completion of the data mining procedure, 31 components had been calculated with associated topographies and waveform activities.

Of the 31 components calculated via data mining, 14 of them were validated as representations of distinct brain activity sources by the volume-domain validation algorithm. These are components 2, 4, 7, 9, 17, 18, 22, 23, 25, 26, 28, 29, 30, and 31.

### Volume Estimation

Estimated brain volume source origins for each of the 14 validated components (FIG. 42) illustrate that numerous brain areas have activities involved this visuospatial navigation paradigm. A visual comparison of the localized brain source volumes to the features of the white matter model onto which the brain volumes have been localized suggests activities in specific cortical areas. The active brain areas of the right hemisphere include the: dorsolateral prefrontal cortex, ventral extrastriate visual cortex, anterior parietal cortex (somatosensory cortex), medial anterior parietal cortex, primary visual striate cortex, posterior parietal cortex, primary motor cortex, superior parietal lobule, and the superior medial parietal lobule. The active brain areas of the left hemisphere include the: dorsolateral prefrontal cortex, primary visual striate cortex, posterior inferior temporal cortex, dorsal extrastriate visual cortex, ventral extrastriate visual cortex, and the superior parietal lobule.

The relative proximities and positions of volume locations of activation suggest the presence of cortical pathways. Notably, the data suggest the presence of a pathway projecting from posterior areas dorsally to prefrontal areas of the cortex in the right hemisphere, generally believed to be involved in processing spatial relationships. The dorsal pathway in the left hemisphere is evidently shorter. Similarly, a ventral pathway is present in both hemispheres, however in the right hemisphere, the ventral pathway is notably shorter. Activity of the ventral pathways has been generally attributed to object perception. The presence of these pathways was anticipated in the introduction when highlighting the various known cortical processing streams.

The locations of components, determined by visual inspection of localization results of components on a model cortex, are summarized in Table 10. Table 10. Component numbers, color reference, possible brain locations. Secondary name or acronym is given in parenthesis. Components were linked to specific brain location descriptions by visual comparison of the localized brain source volumes to the features of the white matter model onto which the brain volumes have been localized.

| Comp. | Color | Hemi. | Location |
|---|---|---|---|
| 2 | blue | L | dorsolateral prefrontal cortex (DLPFC) |
| 4 | blue | R | dorsolateral prefrontal cortex (DLPFC) |
| 7 | cyan | R | ventral extrastriate visual cortex |
| 9 | cyan | L | ventral extrastriate visual cortex |
| 18 | magenta | L | posterior inferior temporal (PIT) |
| 22 | magenta | R | anterior parietal cortex |
| | | | (somatosensory cortex) |
| 23 | green | R | medial anterior parietal cortex, or superior |
| | | | medial post-central gyrus |
| 25 | orange | L | dorsal extrastriate visual cortex |
| 26 | purple | L | superior parietal lobule |
| 28 | red | L + R | primary striate visual cortex |
| 29 | orange | R | posterior parietal cortex (PPC) |
| 30 | pink | R | primary motor cortex |
| 31 | purple | R | superior parietal lobule |
| 17 | black | R | superior medial parietal lobule |

Comparison of the RMS activations of brain areas in the first second of trials is given in FIG. 43 using both non-standardized and standardized RMS data. Evaluation of the difference between conditions by applying ANOVA to non-standardized data (FIG. 43a) does not reveal any significant differences in activation between cue and place conditions for the group. However, differences between the RMS activations for brain areas (FIG. 43b) applying ANOVA to standardized data indicate an effect of condition for some brain area activities. (Standardization of the data as described in the Methods section estimates and removes between participant variability.) In particular, ANOVA reveals a significant effect (p=0.015; F=6.9; Fcrit=4.3) of condition for component number 29 that was found to be located in the posterior parietal cortex of the right hemisphere indicating that this brain area is more active in the place condition than in the cue condition.

Analysis using standardized RMS activations also reveals that components approaching a significant difference between conditions, with more activity in the cue condition than the place condition, are components 17 (superior medial parietal lobule), 4 (dorsolateral prefrontal cortex), 23 (medial anterior parietal cortex), 31 (superior parietal lobule).

Correlation analysis presented in FIG. 44a shows some significant relationships between RMS activation of brain areas and trial completion latencies suggesting activation of these areas relates to poor navigation performance for place trials. This correlation is positive and significant for components 7 (right extrastriate visual cortex), 4 (right dorsolateral prefrontal cortex), 9 (left extrastriate visual cortex) (p<0.05). This result might indicate that some participants navigating place trials were not using the appropriate allocentric strategy but were actually using an inefficient egocentric strategy leading to increased latency to complete the place trials.

FIG. 44a also reveals some possible relationships between activities in specific brain areas and poor place performance that might be revealed as significant in a larger study. For example, components 17 (right superior medial parietal lobe), 2 (left dorsolateral prefrontal cortex), 25 (left dorsal extrastriate visual cortex), and 28 (primary striate visual cortex) are approaching a significant relationship with trial latency in the place condition. In the cue condition, component 26 (left superior parietal lobule) is approaching a significant positive correlation with latency suggesting that activity in this area might contribute to poor navigation in cue trials.

Correlation analysis of brain activation with DS-probe scores calculated across study participants shows no significant correlations but does suggest some possible trends of interest that might be significant in a larger study. In the place condition, activity of component 22 (right anterior parietal cortex) is positively correlated with the measure of explicit knowledge of the platform location. A significant finding here would suggest that when this area is active for place trials, participants can identify the location of the hidden platform when asked. Brain area activities approaching a significant positive correlation with explicit knowledge of platform location in the cue condition include components 4 (right dorsolateral prefrontal cortex), and 9 (left ventral extrastriate visual cortex). This suggests that in the cue condition, if these areas are active, the participant can identify the location of the hidden platform when asked, and presumably, could identify which cue is important.

Component 29 (right posterior parietal cortex) in the cue condition approaches a significant negative correlation with explicit knowledge indicating that a larger study might find that in the cue condition, if a participant can not show where the location of the platform is when asked, they might have been utilizing their right posterior parietal cortex in the task. Conversely, if they can show where the platform is when asked in the cue condition, then the activity of their right posterior parietal cortex is expected to have decreased activity.

The distribution of samples for some pair-wise component relationships suggests significant correlation among activations for different brain areas. These significant correlations indicate consistency across the participant group in the activation characteristics of pairs of brain areas. Scatter plots for a subset of these components are given in FIG. 45. For a correlation to be significant for 12 minus 2 degrees of freedom (for a 12-participant study), the correlation must be greater than or equal to 0.58 for p<0.05.

Significant relationships (p<0.05) for the cue condition were found between components: 9 (left extrastriate visual cortex) and 25 (left dorsal extrastriate visual cortex) (r=0.68), 2 (left dorsolateral prefrontal cortex) and 7 (right extrastriate visual cortex) (r=0.58), 26 (left superior parietal lobule) and 29 (right posterior parietal cortex) (r=0.78), and 9 (left extrastriate visual cortex) and 18 (left posterior inferior temporal cortex) (r=0.80) (FIGS. 45a, b, c, and d, respectively).

For these same components, relationships for the place condition are not all significant. The only significant correlative relationship was between components 2 (left dorsolateral prefrontal cortex) and 7 (right extrastriate visual cortex) (r=0.71). Non-significant correlations were found between components 9 (left extrastriate visual cortex) and 25 (left dorsal extrastriate visual cortex) (r=0.40), 26 (left superior parietal lobule) and 29 (right posterior parietal cortex) (r=0.57), and 9 (left extrastriate visual cortex) and 18 (left posterior inferior temporal cortex) (r=0.42).

In FIGS. 45a, b, and d, participant 7 may or may not to be an outlier in the distribution of samples. Participant 7 has been marked in the figure to demonstrate the scenario where it is regarded as an outlier. With the removal of this participant from the distributions of FIGS. 45a, b and d, the non-significant correlations become significant for 11 minus 2 degrees of freedom (p<0.05; r>0.60). Specifically, component pairs 9 and 25 in the place condition change from r=0.40 to 0.62 (FIG. 45a), component pairs 7 and 2 in the cue condition change from r=0.58 to 0.74 (FIG. 45b), and component pair 9 and 18 in the place condition change from r=0.42 to 0.63 (FIG. 45d).

To emphasize that the activation of some component pairs is more consistent across the participant group than others, FIG. 46 depicts two cases where there is no consistency of activation between components. This is the case for pairs: 29 (right posterior parietal cortex) and 25 (left dorsal extrastriate visual cortex) (FIG. 46a) for both the cue condition (r=0.49) and the place condition(r=0.33), and 29 (right posterior parietal cortex) and 28 (primary visual cortex) (FIG. 46b) for both the cue condition (r=0.54) and the place condition (r=0.20). The distribution of samples for both conditions is ‘box-like’, and is not elongated as would be expected for a correlative relationship.

A summary of the correlative relationships some of which were previously given in scatter plots is given in FIG. 47. The correlative relationships calculated in this way indicate which pairs of components have an activation relationship that is consistent across the participant group and which pairs do not have this relation. Lines that connect blocks in the diagram indicate consistency of the relationship between those blocks across the group of study participants. The degree of consistency indicated by the level of correlation is reflected by the thickness of the line. This representation of pair-wise relationships for all components provides an estimation of the coordination of the brain as a system for each of the behavioural conditions. In the cue condition thick connecting lines dominate the left posterior ventral region, while in the place condition thick connecting lines dominate the right parietal region in the figure.

Zero-lag evaluation of component activities revealed relationships in the time-varying activations of components in the interval around the trial onset. An example of the zero-lag correlation calculated between component pairs is given in FIG. 48 for components 29 and 31 showing a difference between conditions around the interval of trial onset.

A summary of zero-lag correlations for the interval around trial onset (−500 to 500 ms) is plotted on the model cortex of FIG. 49. Connecting lines on the figure indicate components having a significant difference in the level of correlation between conditions. Component pairs with zero-lag correlation having greater correlation for place trials than cue trials are: Left dorsolateral prefrontal cortex (2) and right ventral extrastriate visual cortex (7), right posterior parietal cortex (29) and right superior parietal lobule (31), left dorsal extrastriate visual cortex (25) and right posterior parietal cortex (29), left dorsal extrastriate visual cortex (25) and right superior parietal lobule (31), left posterior inferior temporal cortex (18) and right primary motor cortex (30). Component pairs with zero-lag correlation having greater correlation for cue trials than place trials are: right ventral extrastriate visual cortex (7) and right anterior parietal cortex (22).

### Example Illustrating Correct Operation of Algorithms in a Study of Spatial Navigation

In this example the disclosed technology was used to investigate brain activity while people were playing a video game. In application, once we know how people use their brains to play video games, we can use the disclosed technology to see how they use their brains for other activities like writing essays, singing, playing the piano, taking a quiz, or solving a Rubik's Cube puzzle. We should also be able to see how the activities of our brains change when people consume a pharmaceutical such as a treatment for depression or age-related diseases such as Parkinson's disease.

In the current study, the investigation described above was replicated using a larger number of participants (32) and using a 64-channel electrode EEG system instead of a small number of participants and a 32- channel electrode EEG system.

Participants were asked to find a particular target in a virtual room in one of three conditions. In the first condition (guidance), participants had to go to a target that was visible on the floor in front of them. In the second condition (cue), participants had to go to a target on the floor that was not visible, but was marked by a cue nearby. Because there were 8 different cues in the room, participants had to learn which one marked the target location. In the third condition (place), participants had to go to a target on the floor that was not visible, but could be found by triangulating their position in the room with multiple distal features of the landscape seen through windows of the room. (There is a window on each of the 4 walls that reveal characteristics of the landscape outside the room.) In other words, in the cue and place conditions, based on information in and outside of the room, participants had to imagine where the target was and then go there.

While participants were engaged in the task and while EEG data were recorded, eye-gaze information was also recorded for the purpose of determining what segments of EEG data might reveal important information about brain function related to the navigation task.

Analysis of the eye-gaze data indicated that the first 500 ms is an important time in which to investigate brain function. For example, FIG. 50 shows that there is on average, a difference between the eye-gaze positions between the cue and the place navigation conditions in the first 500 ms of navigation. Hence, the interval 0 to 500 ms was used to analyze brain function activation and coordination.

The results of the EEG analysis indicate that multiple areas of the brain are active while playing a 1st-person videogame. Results were calculated using measures of RMS activity to estimate the level of activation in each brain area and zero-lag correlation was used to estimate coordination among brain areas. The volume regions in FIGS. 51 and 54 indicate regions of activity in the brain whereas the lines connecting regions indicate there is coordination between these areas in the time interval examined.

In FIG. 51, black squares indicate anatomical locations where the activity in the cue navigation condition was significantly greater than activity in the compared to the guidance condition. The results shown indicate that finding your way in a 3D environment (cue navigation) requires more activation and coordination of multiple areas in our right hemisphere than simply moving towards a visible target (guidance). In FIG. 54, the black squares indicate anatomical locations where the activity in the place navigation condition was significantly greater than activity compared to the cue navigation condition. The results shown in this case indicate that a specific ‘additional’ brain system is required to do navigate in the place navigation condition. This brain system revealed is a dorsal projection from the posterior-parietal region of the right hemisphere among some additional activation in other right-hemisphere areas.

These results strongly suggest that the act of finding our way in our world requires the right hemisphere of our brain. What is most important is that, while many studies have indicated that the right side of the brain is important for navigation our results provide a much more detailed picture of the multiple brain areas that are involved. Combining the results of activation and coordination to create a schematic diagram of functional operations reveals how information is processed in the brain. FIG. 52 provides a depiction of the cue condition with the guidance condition used as a baseline. The diagram illustrates that there is a direct link between areas of the brain involved with complex coordinate movement processing, eye-movement, and movement planning. The diagram also illustrates a direct relationship between movement of the hand (to control a joystick), location objects in relation to one's position in space, sensory processing, and decision making. These results demonstrate how the disclosed technology can be used to automatically create block diagrams of the functional operations of the brain and information processing in particular behavioural tasks.

These findings are valuable because they objectively reflect real brain activity and are not biased by pre-conceived ideas about what people believe should be active. Other research investigating different aspects of cognition and behaviour could use the disclosed technology to create 3D brain maps (also referred to as data-driven models of brain function) like the ones in the current investigation (however with appropriate differences related to the cognition and behaviour investigated) and they can be produced the same day that data collection is completed.

### Example Demonstrating Efficacy of Sphereing Matrix

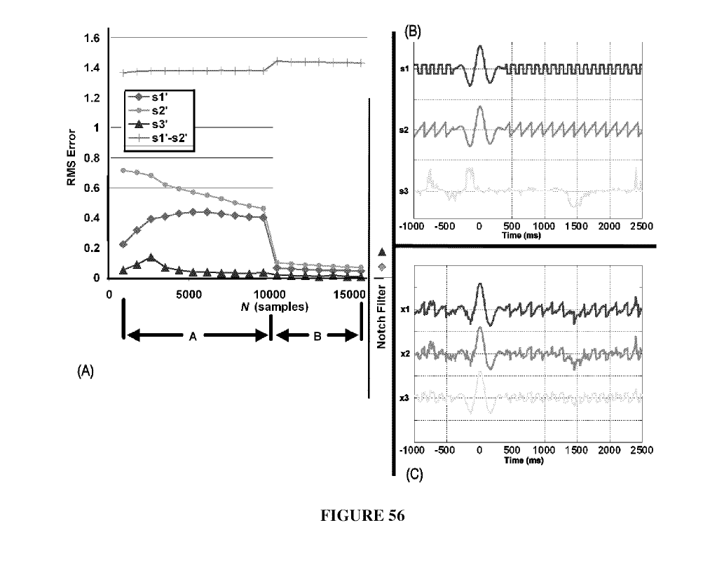

FIG. 56 models the effects of momentary correlations between source activities on source estimates. The source estimates in this example were obtained using runica. The Figure is divided into parts (A), (B), and (C).

Part (B) illustrates sources s₁, s₂, and s₃, that were mixed together to create the observed mixture on sensor channels. Part (C) illustrates the signals measured on the sensor channels x₁, x₂, and x₃ after simulated source signal mixing. In Part (B), the interval of momentary correlation of the source signals is visible as a smooth peak. In the mixture illustrated in Part (C), all three sensor channels exhibit correlated activity as a result of simulated source signal mixing. Part (B) also illustrates samples that are uncorrelated and are distinctly visible from the correlated activity. An analysis of ratio of correlated vs. uncorrelated samples extends the length of uncorrelated samples in these plots. The number of uncorrelated samples is given as a function of N in Plot (A). Plot (A) shows the results of analysis providing: (1) RMS source estimation error as the number of samples uncorrelated activity increases with respect to a momentary high correlation between the activities of sources, and (2) RMS source estimation error upon attenuating activities of correlation using a notch filter (indicated as ‘Notch filter’).

The sources with the momentary high correlation are indicated as s₁′ and s₂′. Source s₃′ has low to no correlation with sources s₁′ and s₂′. The difference between the original s₁ and s₂ and the estimated and s₂′ is plotted as RMS error illustrates a sudden transition at approximately 10000 samples. This transition is also reflected in the general plot of RMS error of s₁′ and s₂′. The results presented in Plot (A) illustrate two main ideas: (1) as the number of uncorrelated signal samples increases with respect to the number of correlated samples, the source estimation error decreases, and (2) removing the frequencies of correlated activity using a filter reduces the source estimation error. An important effect of momentary correlated activity and non-stationary signals as they are summarized by a measure of covariance is depicted in the sudden transition of RMS error around 10000 samples in the plot.

The calculation of covariance (also known as zero-mean correlation) and hence the mixing matrix derived from the estimate of covariance completely misallocates s₁′ and s₂′ when the interval of momentary correlation and the associated variance is large compared with the interval of uncorrelated activity. In other words, the momentary correlation is dominating the covariance matrix. However, when enough uncorrelated samples are added or the variance of these uncorrelated samples is large enough, the covariance matrix is no-longer dominated by the momentary correlation and the misallocation of s₁′ and s₂′ is addressed. Stated another way, because of the error in the covariance matrix due to the momentary correlation, sources s₁′ and s₂′ are not identified as distinct sources. When the error in the covariance matrix is mitigated by reducing the effect of the correlation on the estimate of covariance, s₁′ and s₂′ are identified as distinct from each other. Attenuation of activities in the frequency bands at which correlated activities exist between sources (provided these sources have activities in frequency bands that are not correlated), will reduce the variance related to these correlated activities and can lead to a more accurate identification of distinct sources.

Certain implementations of the invention comprise computer processors which execute software instructions which cause the processors to perform a method of the invention. For example, one or more processors in a neurological testing device, a neurological feedback device may implement the methods as described herein by executing software instructions in a program memory accessible to the processors. The invention may also be provided in the form of a program product. The program product may comprise any medium which carries a set of computer-readable signals comprising instructions which, when executed by a data processor, cause the data processor to execute a method of the invention. Program products according to the invention may be in any of a wide variety of forms. The program product may comprise, for example, physical media such as magnetic data storage media including floppy diskettes, hard disk drives, optical data storage media including CD ROMs, DVDs, electronic data storage media including ROMs, flash RAM, or the like. The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

As will be apparent to those skilled in the art in the light of the foregoing disclosure, many alterations and modifications are possible in the practice of this invention without departing from the spirit or scope thereof. For example:
Features of embodiments described above may be combined in different ways with one another and with other technology to produce additional embodiments. Many permutations and combinations are possible.

1. A method for monitoring activity of modular sources within the brain, the method comprising:
obtaining encephalography data for one or more subjects;
processing the encephalography data using a first independent component analysis algorithm to identify a plurality of first components within the encephalography data;
based on the first components, defining a spectral shaping filter emphasizing spectral frequencies at which the components of the first plurality of components are more statistically independent over other frequencies at which the components of the first plurality of components are less statistically independent;
processing the encephalography data using the spectral shaping filter to yield spectrally-shaped encephalography data;
processing the spectrally-shaped encephalography data using a second independent component analysis algorithm to identify a plurality of second components within the spectrally-shaped encephalography data; and,
processing the encephalography data to determine weights corresponding to the second components.

2. A method according to claim 1 wherein the encephalography data comprises electroencephalography (EEG) data.

3. A method according to claim 1 wherein the encephalography data comprises magnetoencephalography (MEG) data.

4. A method according to claim 1 wherein the first independent component analysis algorithm comprises determining a preliminary weight matrix W and a sphering matrix P from the encephalography data.

5. A method according to claim 4 wherein the first independent component analysis algorithm further comprises performing a band-specific independent component analysis algorithm using the weight matrix W, sphering matrix P, and encephalography data as inputs to the band-specific independent component analysis algorithm.

6. A method according to claim 1 comprising processing the second components to yield volume domain characteristics of the second components.

7. (canceled)

8. A method according to claim 6 wherein processing a second component to yield volume domain characteristics comprises synthesizing encephalography data corresponding to the second component and providing the synthesized encephalography data to a beamforming algorithm.

9. A method according to claim 8 wherein synthesizing encephalography data comprises multiplying a synthetic time-varying waveform with a topography corresponding to the second component.

10. A method according to claim 9 wherein synthesizing encephalography data comprises adding synthetic noise wherein the synthetic noise on all dimensions has zero correlation and the synthetic signal has zero correlation with the synthetic noise.

11. A method according to claim 8 wherein the beamforming algorithm comprises a LCMV beamformer.

12. A method according to claim 1 wherein the second independent component analysis algorithm is iterative, the method comprises performing a plurality of iterations of the second independent component analysis algorithm and, for the iterations:
recording volume domain characteristics corresponding to one of the second components;
determining differences between the volume domain characteristics for the second component for different iterations; and,
based at least in part on the differences identifying the one second component as corresponding to an artifact.

13. A method according to claim 1 wherein the encephalography data comprises a plurality of channels, processing the encephalography data to determine weights is performed for each of a plurality of time slots to obtain a plurality of time-varying source value signals corresponding to the second elements.

14.-16. (canceled)

17. A method according to claim 13 wherein processing the encephalography data to determine weights corresponding to the second components comprises, for each of the plurality of time slots matrix multiplying a vector having elements corresponding to the channels of the encephalography data by a matrix to yield a corresponding vector of source values.

18. A method according to claim 1 wherein the encephalography data comprises a plurality of channels and processing the encephalography data to determine weights corresponding to the second components comprises, for each of a plurality of time slots matrix multiplying a vector having elements corresponding to the channels of the encephalography data by a matrix to yield a corresponding vector of source values.

19.-25. (canceled)

26. A method for processing encephalography data to remove artifacts, the method comprising:
processing the encephalography data using an independent component analysis algorithm to identify a plurality of components;
processing the components to yield one or more volume domain characteristics of the components; and
comparing the one or more volume domain characteristics to corresponding thresholds.

27. A method according to claim 26 wherein the volume domain characteristics comprise one or more of: peak spectral value (PSV); average volume overlap of components (AVO); median volume overlap of components (MVO); average volume overlap absolute (AVO′) and median volume overlap absolute (MVO′).

28. A method according to claim 27 wherein the independent component analysis algorithm is iterative, the method comprises performing a plurality of iterations of the independent component analysis algorithm and, for the iterations:
recording volume domain characteristics corresponding to one of the components;
determining differences between the volume domain characteristics for the one component for different iterations; and,
based at least in part on the differences identifying the one component as corresponding to an artifact.

29.-37. (canceled)

38. Apparatus for monitoring activity of modular sources within the brain, the apparatus comprising:
an data store containing encephalography data for one or more subjects;
a processor configured for:
processing the encephalography data using a first independent component analysis algorithm to identify a plurality of first components within the encephalography data;
based on the first components, defining a spectral shaping filter emphasizing spectral frequencies at which the components of the first plurality of components are more statistically independent over other frequencies at which the components of the first plurality of components are less statistically independent;
processing the encephalography data using the spectral shaping filter to yield spectrally-shaped encephalography data;
processing the spectrally-shaped encephalography data using a second independent component analysis algorithm to identify a plurality of second components within the spectrally-shaped encephalography data; and,
processing the encephalography data to determine weights corresponding to the second components.

39. Apparatus method according to claim 38 wherein the first independent component analysis algorithm comprises determining a preliminary weight matrix W and a sphering matrix P from the encephalography data.

40. A method according to claim 39 wherein the first independent component analysis algorithm further comprises performing a band-specific independent component analysis algorithm using the weight matrix W, sphering matrix P, and encephalography data as inputs to the band-specific independent component analysis algorithm.

41.-60. (canceled)
